(19) United States
(12) Patent Application Publication
(10) Pub. No.: US 2026/0258099 A1
(43) Pub. Date: Sep. 3, 2026

(54) IG-LIKE FUSION PROTEINS FOR TREATING PEMPHIGUS VULGARIS AND FOLIACEUS

(71) Applicant: CANOPY IMMUNO-THERAPEUTICS LTD., Moshav Megadim (IL)

(72) Inventors: Kfir OVED, Moshav Megadim (IL); Galit DENKBERG, Nofit (IL); Sharon REEF, Tzukei Yam (IL); Yelena PINZUR, Haifa (IL); Inbar ARMAN ZELMAN, Nesher (IL)

(21) Appl. No.: 19/165,135

(22) PCT Filed: Mar. 13, 2024
(86) PCT No.: PCT/IL2024/050269

§ 371 Date: Sep. 14, 2025

Related U.S. Application Data
(60) Provisional application No. 63/451,668, filed on Mar. 13, 2023.
(60) Provisional application No. 63/609,419, filed on Dec. 13, 2023.

(51) Int. Cl. C07K 14/47; A61K 38/00; A61K 47/64; A61P 37/06; G01N 33/564

(52) U.S. Cl. CPC C07K 14/47; A61K 47/64; A61P 37/06; G01N 33/564; A61K 38/00; G01N 2800/52

(57) ABSTRACT

Compositions comprising a first polypeptide comprising a first fragment of an extracellular domain of DSG1 or DSG3 or an analog or derivative thereof and a dimerization domain and a second polypeptide comprising a second fragment of an extracellular domain of DSG1 or DSG3 or an analog or derivative thereof and a dimerization domain are provided. Polypeptides comprising fragments of an extracellular domain of DSG1 or DSG3 comprising at least one mutation that increases solubility, decreases aggregation or both or which is devoid of extracellular domains 3 and 4 are also provided. Pharmaceutical compositions comprising the composition, polypeptide, nucleic acid systems and molecules encoding the polypeptides of the composition and invention and methods of treatment and determining suitability for treatment using the compositions or polypeptides; as well as methods of producing the compositions or proteins are also provided.

### CROSS REFERENCE TO RELATED APPLICATIONS

This application claims the benefit of priority of U.S. Provisional Patent Application No. 63/451,668, filed on Mar. 13, 2023, and entitled “IG-LIKE FUSION PROTEINS AND USE THEREOF”, and U.S. Provisional Patent Application No. 63/609,419, filed on Dec. 13, 2023, and entitled “IG-LIKE FUSION PROTEINS FOR TREATING PEMPHIGUS VULGARIS AND FOLIACEUS”, the contents of which are all incorporated herein by reference in their entirety.

### REFERENCE TO AN ELECTRONIC SEQUENCE LISTING

The contents of the electronic sequence listing (CNPY-P-009-PCT.xml; Size: 257,833 bytes; and Date of Creation: Feb. 19, 2024) is herein incorporated by reference in its entirety.

### FIELD OF INVENTION

The present invention is in the field of fusion protein generation and Pemphigus vulgaris and Pemphigus foliaceus treatment.

### BACKGROUND OF THE INVENTION

Pemphigus vulgaris/foliaceus (PV/PF) are rare, life-threatening autoimmune blistering diseases that affect the skin and mucus. In the majority of cases, PV initially presents with oral mucosa lesions, specifically in areas exposed to frictional trauma, such as the buccal and lateral tongue mucosa. The lesions begin as vesicles which easily rupture and leave behind erosions and ulcers. Many patients will then go on to develop skin lesions over the following weeks or months. PF has a similar skin distribution as that of PV, but mucosal surfaces are unaffected.

Pemphigus vulgaris occurs worldwide with incidence rates between 0.1 and 2.7 per 100,000 people per year and a prevalence of 5.2/100,000. While Pemphigus vulgaris occurs in all racial and ethnic groups, the highest incidence is seen in Ashkenazi Jews of Mediterranean origin. Average age of onset is between 40-60 years old with a close to equivalent sex ratio.

Pathophysiology: PV and PF are caused by IgG1 and IgG4 autoantibodies to the keratinocyte adhesion proteins Desmoglein-1 (Dsg-1) and Desmoglein-3 (Dsg-3) which comprise desmosomes. Binding of the autoantibodies compromises intraepidermal adhesion and causes acantholysis resulting in the formation of intraepithelial blisters in mucous membranes and skin. Pemphigus vulgaris is characterized by mucosal and cutaneous involvement with autoantibodies against Dsg-3 (mucosal) or both Dsg-3 and Dsg-1 (mucocutaneous). Pemphigus foliaceus is characterized by cutaneous involvement with autoantibodies against Dsg-1 alone.

Dsg-1 and Dsg-3 are 160 and 130 KDa transmembrane glycoproteins, respectively, and a part of the cadherin family. Structurally, they contain an extracellular domain with five structural domains (EC1-EC5), a transmembrane domain, and cytoplasmic domains. The distal extracellular domains of the cadherin molecule (amino-terminal EC1 and EC2 domains) bind to the corresponding distal domains of the adjacent cell membrane, and it is this connection which is targeted and disrupted by the autoantibodies.

While there are four Desmoglein isoforms, Dsg-1 and Dsg-3 are the only ones expressed in squamous stratified epithelium, whereas Dsg-2 is expressed in simple epithelium and myocardium, and Dsg-4 is expressed in hair follicles. It is still unclear whether additional proteins which comprise the desmosome, such as Desmocollins, play a role in the pathogenesis of Pemphigus, and why they fail to compensate for the loss of Desmoglein function.

It has been shown that during the acute phase of the disease, IgG4 predominates, while periods of remission are associated with IgG1. While several hypotheses have developed, what triggers autoantibody production and the mechanism by which the binding of autoantibodies leads to acantholysis still remains uncertain. Such theories include alterations in intracellular transduction signaling and separation of the cytoskeleton, resulting in keratinocyte shrinkage; spatial hindrance for Desmoglein adhesion; and formation of Desmoglein-deficient desmosomes.

The pathogenic role of anti-Dsg antibodies has been well-established in several studies. In-vitro studies have shown that when IgG from PV patient serum was added to cultured keratinocytes, there was a transient increase in intracellular signaling molecules. This induced the phosphorylation and dissociation of Dsg3 as well as keratin retraction from cell-cell contact sites. In-vivo studies have shown that the passive transfer of IgG antibodies from serum of patients with PV into mice induces blistering with features consistent with pemphigus. It is believed that the N-terminal extracellular domain 1 (EC1) of DSG3 is essential for the binding of DSG3 and mouse monoclonal antibodies targeting this domain have induced experimental PV in vivo. However, in humans, the targeting of DSG3 is more complex than in mice, with PV patients producing polyclonal antibodies targeting the extracellular domain 1 epitope that can be overcome by p38 MAPK inhibition, suggesting that antibody effects on signaling might play an important role beyond just physical blockade.

Diagnosis and Treatment. Diagnosis is made through clinical findings and confirmatory lab investigations including biopsy of skin lesions and serology. However, due to lack of awareness and failure to recognize the clinical features, it takes on average 5 doctors and 10 months to confirm a diagnosis. A tissue biopsy and direct immunofluorescence remains the best diagnostic tool in combination with clinical findings, while indirect immunofluorescence and ELISA play a complementary role. More than 80% of patients with active disease produce IgG1 and IgG4 Dsg-1 and Dsg-3 specific autoantibodies that are detectable by indirect immunofluorescence. FDA-approved ELISA tests are commercially available as well, with high sensitivity and specificity. Disease activity correlates with antibody titers in most cases and the use of ELISA aids with monitoring disease activity and treatment response.

Before systemic corticosteroids were introduced, the prognosis of PV was almost always fatal within the first 2 years following initial presentation. With proper treatment, the mortality rate of PV has drastically reduced, yet still remains at approximately 15%. Most deaths are considered to be a result from complications of treatment, due to secondary effects of the first- and second-line immunosuppressive agents.

First line treatment includes Anti-CD20 antibodies in combination with systemic corticosteroids to attain rapid control of disease activity (reduce lesion appearance) within several weeks and is followed by slowly tapering steroid dosage. Unfortunately, due to the chronicity of the disease, long term systemic corticosteroid therapy is necessary to maintain the clinical response and comes with an increased incidence of serious adverse events, including severe infections due to the immunocompromised state, diabetes mellitus, osteoporosis, hypertension, and gastrointestinal ulcers.

While the majority of patients can achieve initial improvement with first-line therapy, other patients fail to respond or experience frequent relapses. Major therapeutic options for refractory disease involve directly targeting the autoantibody-mediated response through the use of IVIG, plasmapheresis, or Rituximab. Recently, Rituximab was introduced as an adjuvant to patients who are refractory to first-line steroids and also as a steroid sparing addition to first-line therapy. 65% of patients remain in remission (from 3-22 months) with the use of Rituximab in addition to immunosuppressive therapy. The overall incidence of severe adverse effects to rituximab is 1-16% in various studies and includes infection frequently resulting in septicemia. Today, systemic glucocorticoids and Rituximab are the mainstays of therapy for PV and PF and are usually highly effective in controlling the disease. However, safer and more durable therapies are necessary due to life-threatening adverse effects associated with the first-line therapies.

Primary endpoints in clinical studies include the proportion of patients who achieved sustained complete remission off-therapy for up to an extended time period and efficacy in reducing Pemphigus Disease Area Index (PDAI) scores within a few weeks compared to baseline. In general, the trials compare the novel treatment modality to first-line steroid treatment or a placebo following initial steroid treatment.

Surrogate and Predictive Biomarkers. Antibody titer is a surrogate measure that has reportedly correlated to disease activity in Pemphigus. Higher risk of overall mortality is associated with age of onset≥65 years, the presence of coronary heart disease or cardiac arrhythmias, and higher levels of anti-DSG1 and/or anti-DSG3 antibodies (≥100 U/mL). Similarly, a correlation between increased levels of anti-DSG1 and/or anti-DSG3 autoantibodies and higher disease activity has been reported. This is in line with the fact that titers of anti-DSG1 and/or anti-DSG3 antibodies are associated with the more severe, mucocutaneous form of PV, and respond more poorly to treatment than patients with the mucous or cutaneous form of PV. Improved therapeutic modalities for treating PV/PF are greatly needed.

### SUMMARY OF THE INVENTION

The present invention provides polypeptides comprising fragments of an extracellular domain of desmoglein-1 (DSG1) or desmoglein-3 (DSG3) comprising at least one mutation that increases solubility, decreases aggregation or both or which is devoid of extracellular domains 3 and 4 and an effector moiety that is not an unmodified Fc domain. The invention further provides compositions comprising a fragment of a first human receptor target of Pemphigus vulgaris autoantibodies and a fragment of a second human receptor target of Pemphigus vulgaris autoantibodies and an effector moiety that is not an unmodified Fc domain. Methods of treating Pemphigus vulgaris or Pemphigus foliaceus by administering a pharmaceutical composition of the invention are also provided, as are nucleic acid molecules and systems encoding the polypeptides and compositions of the invention, methods of producing those polypeptides and compositions and methods of determining suitability to be treated by a method of the invention.

According to a first aspect, there is provided a polypeptide comprising a fragment of an extracellular domain of Desmoglein 1 (DSG1) or Desmoglein 3 (DSG3) and at least one mutation that increases solubility of the fragment, decreases aggregation of the fragment or both.

According to another aspect, there is provided a polypeptide of the invention comprising an effector moiety. In some embodiments, the effector moiety is an effector moiety that is not an unmodified Fc domain.

According to some embodiments, the at least one mutation is in a cadherin domain or a calcium binding domain.

According to some embodiments, the DSG1 extracellular domain comprises SEQ ID NO: 2 and the mutation is selected from: W2A, D27T, E89S, A80I, K17A, R18A, RIOA, R97A, C9V, C9S, C28A, C28S, C78V, C78S, C204A, C204S, C206V, C206S, C426S, E69G, D103G, D136G, E179G, D215G, D247G, E231G, E291G, E332G, N61Q, and N131Q.

According to some embodiments, the DSG1 extracellular domain comprises SEQ ID NO: 2 and the mutation is selected from: W2A, D27T, E89S, A80I, K17A, R18A, R10A, R97A, C9V, C28A, C78V, C204A, C206V, C426S, E69G, D103G, D136G, E179G, D215G, D247G, E231G, E291G, E332G, N61Q, and N131Q.

According to some embodiments, the at least one mutation is:
a. W2A;
b. D27T;
c. E89S;
d. W2A, D27T and E89S;
e. W2A and A80I;
f. K17A and R18A;
g. R10A, K17A, R18A, and R97A;
h. C9V, C28A, C78V, C204A and C206V;
i. C9V, C28A, C78V, C204A, C206V and C426S;
j. E69G, D103G, and D136G;
k. E179G, D215G and D247G;
l. E231G, E291G, and E332G;
m. N61Q and N131Q; or
n. C9S, C28S, C78S, C204S, and C206S.

According to some embodiments, the at least one mutation is:
a. W2A;
b. D27T;
c. E89S;
d. W2A, D27T and E89S;
e. W2A and A80I;
f. K17A and R18A;
g. R10A, K17A, R18A, and R97A;
h. C9V, C28A, C78V, C204A and C206V;
i. C9V, C28A, C78V, C204A, C206V and C426S;
j. E69G, D103G, and D136G;
k. E179G, D215G and D247G;
l. E231G, E291G, and E332G; or
m. N61Q and N131Q.

According to some embodiments, the DSG3 extracellular domain comprises SEQ ID NO: 4 and the mutation is selected from: W2A, D27T, E89S, A80I, K17A, R18A, R10A, K97A, C9V, C9S, C78V, C78S, C202A, C202S, C204V, C204S, E69G, D103G, D136G, E179G, D213G, D245G, E229G, E289G, E330G, N61Q, and N131Q.

According to some embodiments, the DSG3 extracellular domain comprises SEQ ID NO: 4 and the mutation is selected from: W2A, D27T, E89S, A80I, K17A, R18A, R10A, K97A, C9V, C78V, C202A, C204V, E69G, D103G, D136G, E179G, D213G, D245G, E229G, E289G, E330G, N61Q, and N131Q.

According to some embodiments, the at least one mutation is:
a. W2A;
b. E89S;
c. W2A, D27T and E89S;
d. W2A and A80I;
e. K17A and R18A;
f. R10A, K17A, R18A, and K97A;
g. C9V, C78V, C202A and C204V;
h. E69G, D103G, and D136G;
i. E179G, D213G and D245G;
j. E229G, E289G, and E330G;
k. N61Q and N131Q; or
l. C9S, C78S, C202S, and C204S.

According to some embodiments, the at least one mutation is:
a. W2A;
b. E89S;
c. W2A, D27T and E89S;
d. W2A and A80I;
e. K17A and R18A;
f. R10A, K17A, R18A, and K97A;
g. C9V, C78V, C202A and C204V;
h. E69G, D103G, and D136G;
i. E179G, D213G and D245G;
j. E229G, E289G, and E330G; or
k. N61Q and N131Q.

According to some embodiments, the fragment consists of extracellular domains (ECs) 1 and 2 of DSG1 or DSG3 and the polypeptide is devoid of extracellular domains (ECs) 3 and 4 of DSG1 or DSG3 and wherein EC1 of DSG1 consists of SEQ ID NO: 114, EC2 of DSG1 consists of SEQ ID NO: 115, EC3 of DSG1 consists of SEQ ID NO: 116, EC4 of DSG1 consists of SEQ ID NO: 117, EC1 of DSG3 consists of SEQ ID NO: 118, EC2 of DSG3 consists of SEQ ID NO: 119, EC3 of DSG3 consists of SEQ ID NO: 120 and EC4 of DSG3 consists of SEQ ID NO: 121.

According to some embodiments, the fragment comprises or consists of an amino acid sequence selected from SEQ ID NO: 74-85, 90-103 and 108-113.

According to some embodiments, the fragment comprises or consists of an amino acid sequence selected from SEQ ID NO: 81, 82, 90, 100 and 108.

According to another aspect, there is provided a polypeptide comprising a fragment of an extracellular domain of Desmoglein 1 (DSG1) or Desmoglein 3 (DSG3) wherein the polypeptide is devoid of extracellular domains 3 and 4 of DSG1 or DSG3 wherein EC3 of DSG1 consists of SEQ ID NO: 116, EC4 of DSG1 consists of SEQ ID NO: 117, EC3 of DSG3 consists of SEQ ID NO: 120 and EC4 of DSG3 consists of SEQ ID NO: 121.

According to some embodiments, the fragment comprises or consists of an amino acid sequence selected from SEQ ID NO: 87, 90-91, 105, and 108-109.

According to some embodiments, the fragment comprises or consists of an amino acid sequence selected from SEQ ID NO: 90 and 108.

According to some embodiments, the polypeptide further comprises an effector moiety.

According to some embodiments, the effector moiety is not an Fc domain.

According to some embodiments, the effector moiety is an Fc domain comprising at least one mutation that increases antibody dependent cell cytotoxicity (ADCC) or complement dependent cytotoxicity (CDC).

According to some embodiments, the effector moiety is capable of inducing death in a cell binding the fragment.

According to some embodiments, the effector moiety is selected from an Fc domain comprising at least one mutation that increases ADCC, an amatoxin/amanitin, an anthracycline, an anthramycin-based dimer, a calicheamicin, camptothecin or an analog thereof, a duocarmycin, triptolide and a tubulin inhibitor.

According to some embodiments, the effector moiety is selected from: alpha-amanitin, PNU-159682, tesirine, deruxtecan (Dxd), mertansine, monomethyl auristatin E (MMAE), monomethyl auristatin F (MMAF) and a combination thereof.

According to some embodiments, the effector moiety is an Fc domain comprising SEQ ID NO: 60 or SEQ ID NO: 62 comprising a plurality of mutations selected from: L15V/F23L/R72P/Y80L/P176L, S19D/A110L/1112E, G16A/A110L/1112E, and G16A/S47E/H48F/S104T/1112E within the SEQ ID NO: 60 or SEQ ID NO: 62.

According to some embodiments, the effector moiety is conjugated to the polypeptide by a linker.

According to another aspect, there is provided a composition, comprising:
a. a first polypeptide comprising a fragment of an extracellular domain of Desmoglein 1 (DSG1) or an analog or derivative thereof, a fragment of an extracellular domain of Desmoglein 3 (DSG3) or an analog or derivative thereof or both and a first dimerization domain; and
b. a second polypeptide comprising a fragment of DSG1 or an analog or derivative thereof, a fragment of DSG3 or an analog or derivative thereof or both and a second dimerization domain;
wherein the first and second dimerization domains are configured to dimerize with each other.

According to some embodiments, the dimerizing comprises forming a covalent bond between the first dimerization domain and the second dimerization domain.

According to some embodiments, the protein complex comprises an immunoglobulin scaffold.

According to some embodiments,
a. the first dimerization domain comprises a first hinge domain of a heavy chain of an immunoglobulin and the second dimerization domain comprises a second hinge domain of a heavy chain and the first and the second dimerization domains dimerizes by a disulfide bond; or
b. the first and second dimerization domains each comprise a domain selected from a CH1 domain of a heavy chain of an immunoglobulin and a CL domain of a light chain of an immunoglobulin and dimerize by a disulfide bond and wherein the first and second dimerization domains do not both comprise the CH1 domain or the CL domain.

According to some embodiments, the fragment and the dimerization domain of the first, second or both polypeptide chains are separated by a linker.

According to some embodiments, the first polypeptide chain, the second polypeptide chain or both further comprise an Fc region of a human antibody heavy chain.

According to some embodiments, the Fc region is capable of inducing cytotoxicity against a cell binding the protein complex.

According to some embodiments, the first polypeptide chain comprises a first CH3 domain of a heavy chain of an immunoglobulin, a first CH2 domain of a heavy chain of an immunoglobulin or both and the second polypeptide chain comprises a second CH3 domain of a heavy chain of an immunoglobulin, a second CH2 domain of a heavy chain of an immunoglobulin or both.

According to some embodiments, the first CH3 domain comprises at least a first mutation and the second CH3 domain comprises at least a second mutation, and wherein the mutations permit heterodimerization of the first and second polypeptide chains and inhibit homodimerization of the first polypeptide chain and homodimerization of the second polypeptide chain.

According to some embodiments, the first CH2 domain comprises at least a first mutation and the second CH2 domain comprises at least a second mutation, and wherein the mutations permit heterodimerization of the first and second polypeptide chains and inhibit homodimerization of the first polypeptide chain and homodimerization of the second polypeptide chain.

According to some embodiments, the first mutation is selected from a mutation provided in Table 1 and the second mutation is provided in Table 1 and is a corresponding mutation to the first mutation.

According to some embodiments, the first mutation is a T366W mutation within a CH3 domain and the second mutation is a combination of aT366S mutation a L368A mutation and a Y407V mutation.

According to some embodiments, the Fc region of the first, second or both polypeptide chains is separated from the fragment or the dimerization domain by a linker.

According to some embodiments, the Fc region comprises at least one mutation that increases ADCC or CDC.

According to some embodiments, the Fc region is an Fc region comprising SEQ ID NO: 60 or SEQ ID NO: 62 comprising a plurality of mutations selected from: L15V/F23L/R72P/Y80L/P176L, S19D/A110L/1112E, G16A/A110L/1112E, and G16A/S47E/H48F/S104T/1112E within the SEQ ID NO: 60 or SEQ ID NO: 62.

According to some embodiments, the dimerization domain of the first, second or both polypeptide chains is C-terminal to the fragment and N-terminal to the Fc region.

According to some embodiments, the composition is devoid of an antibody variable domain.

According to some embodiments, the composition further comprises a third polypeptide comprising a fragment of DSG1 or an analog or derivative thereof, a fragment of DSG3 or an analog or derivative thereof or both and a third dimerization domain, wherein the first polypeptide further comprises a fourth dimerization domain and the third and fourth dimerization domains are capable of dimerizing to each other.

According to some embodiments,
a. the third dimerization domain comprises a first hinge domain of a heavy chain of an immunoglobulin and the fourth dimerization domain comprises a second hinge domain of a heavy chain and the first and the second dimerization domains dimerizes by a disulfide bond; or
b. the third and fourth dimerization domains each comprise a domain selected from a CH1 domain of a heavy chain of an immunoglobulin and a CL domain of a light chain of an immunoglobulin and dimerize by a disulfide bond and wherein the first and third polypeptides do not both comprise the CH1 domain or the CL domain.

According to some embodiments, the composition further comprises a fourth polypeptide comprising a fragment of DSG1 or an analog or derivative thereof, a fragment of DSG3 or an analog or derivative thereof or both and a fifth dimerization domain, wherein the second polypeptide further comprises a sixth dimerization domain and the fifth and six dimerization domains are capable of dimerizing to each other.

According to some embodiments,
a. the fifth dimerization domain comprises a first hinge domain of a heavy chain of an immunoglobulin and the sixth dimerization domain comprises a second hinge domain of a heavy chain and the first and the second dimerization domains dimerizes by a disulfide bond; or
b. the fifth and six dimerization domains each comprise a domain selected from a CH1 domain of a heavy chain of an immunoglobulin and a CL domain of a light chain of an immunoglobulin and dimerize by a disulfide bond and wherein the first and third polypeptides do not both comprise the CH1 domain or the CL domain.

According to some embodiments, the first polypeptide and the second polypeptide do not both comprise a CH1 domain or both comprise a CL domain.

According to some embodiments, the third and fourth dimerization domains or the fifth and sixth dimerization domains comprise mutations that permits dimerization of the third and fourth dimerization domains and the fifth and sixth dimerization domains and inhibit dimerization of the third dimerization domain to the fifth or sixth dimerization domain and the sixth dimerization domain to the third or fourth dimerization domain.

According to some embodiments, the first polypeptide comprises a fragment of DSG1 or an analog or derivative thereof, and the second polypeptide comprises a fragment of DSG3 or an analog or derivative thereof.

According to some embodiments, the first polypeptide chain or the second polypeptide chain comprises both a fragment of DSG1 or an analog or derivative thereof, and a fragment of DSG3 or an analog or derivative thereof.

According to some embodiments, the fragments are separated by an amino acid linker.

According to some embodiments, the DSG1 lacks a prodomain and comprises or consists of SEQ ID NO: 2 and the DSG3 lacks a prodomain and comprises or consists of SEQ ID NO: 4; or the DSG1 comprises a prodomain and comprises or consist of SEQ ID NO: 1 and the DSG3 comprises a prodomain and comprises or consists of SEQ ID NO: 3.

According to some embodiments, the fragment of an extracellular domain consists of a truncation of the extracellular domain lacking an extracellular functional domain comprises a sequence selected from SEQ ID NO: 114-121.

According to some embodiments, the fragment is devoid of extracellular domains 3 and 4 of DSG1 or DSG3 wherein EC3 of DSG1 consists of SEQ ID NO: 116, EC4 of DSG1 consists of SEQ ID NO: 117, EC3 of DSG3 consists of SEQ ID NO: 120 and EC4 of DSG3 consists of SEQ ID NO: 121.

According to some embodiments, the fragment comprises or consists of extracellular domains (ECs) 1 and 2 of DSG1 or DSG3 or an analog or derivative thereof and wherein EC1 of DSG1 consists of SEQ ID NO: 114, EC2 of DSG1 consists of SEQ ID NO: 115, EC1 of DSG3 consists of SEQ ID NO: 118, and EC2 of DSG3 consists of SEQ ID NO: 119.

According to some embodiments, the fragment consists of an amino acid sequence selected from SEQ ID NO: 87, 90-91, 105, and 108-109 or a derivative thereof comprising at least 85% identity.

According to some embodiments, the fragment consists of an amino acid sequence selected from SEQ ID NO: 90 and 108 or a derivative thereof comprising at least 85% identity.

According to some embodiments, at least one of the fragments comprise a mutation in a cadherin domain, or a calcium binding domain.

According to some embodiments, the mutation increases solubility of the composition, decreases aggregation of the complex or both.

According to some embodiments, the DSG1 extracellular domain comprises SEQ ID NO: 2 and the mutation is selected from: W2A, D27T, E89S, A80I, K17A, R18A, R10A, R97A, C9V, C9S, C28A, C28S, C78V, C78S, C204A, C204S, C206V, C206S, C426S, E69G, D103G, D136G, E179G, D215G, D247G, E231G, E291G, E332G, N61Q, and N131Q or the DSG3 extracellular domain comprises SEQ ID NO: 4 and the mutation is selected from: W2A, D27T, E89S, A80I, K17A, R18A, R10A, K97A, C9V, C9S, C78V, C78S, C202A, C202S, C204V, C204S, E69G, D103G, D136G, E179G, D213G, D245G, E229G, E289G, E330G, N61Q, and N131Q.

According to some embodiments, the DSG1 extracellular domain comprises SEQ ID NO: 2 and the mutation is selected from: W2A, D27T, E89S, A80I, K17A, R18A, R10A, R97A, C9V, C28A, C78V, C204A, C206V, C426S, E69G, D103G, D136G, E179G, D215G, D247G, E231G, E291G, E332G, N61Q, and N131Q or the DSG3 extracellular domain comprises SEQ ID NO: 4 and the mutation is selected from: W2A, D27T, E89S, A80I, K17A, R18A, R10A, K97A, C9V, C78V, C202A, C204V, E69G, D103G, D136G, E179G, D213G, D245G, E229G, E289G, E330G, N61Q, and N131Q.

According to some embodiments, the at least one mutation is:
a. W2A in SEQ ID NO: 2 or 4;
b. D27T in SEQ ID NO: 2;
c. E89S in SEQ ID NO: 2 or 4;
d. W2A, D27T and E89S in SEQ ID NO: 2 or 4;
e. W2A and A80I in SEQ ID NO: 2 or 4;
f. K17A and R18A in SEQ ID NO: 2 or 4;
g. R10A, K17A, R18A, and R97A in SEQ ID NO: 2;
h. R10A, K17A, R18A, and K97A in SEQ ID NO: 4;
i. C9V, C28A, C78V, C204A and C206V in SEQ ID NO: 2;
j. C9V, C78V, C202A and C204V in SEQ ID NO: 4;
k. C9V, C28A, C78V, C204A, C206V and C426S in SEQ ID NO: 2;
l. E69G, D103G, and D136G in SEQ ID NO: 2 or 4;
m. E179G, D215G and D247G in SEQ ID NO: 2;
n. E179G, D213G and D245G in SEQ ID NO: 4;
o. E231G, E291G, and E332G in SEQ ID NO: 2;
p. E229G, E289G, and E330G in SEQ ID NO: 4;
q. N61Q and N131Q in SEQ ID NO: 2 or 4;
r. C9S, C28S, C78S, C204S, and C206S in SEQ ID NO: 2; or
s. C9S, C78S, C202S, and C204S in SEQ ID NO: 4.

According to some embodiments, the at least one mutation is:
a. W2A in SEQ ID NO: 2 or 4;
b. D27T in SEQ ID NO: 2;
c. E89S in SEQ ID NO: 2 or 4;
d. W2A, D27T and E89S in SEQ ID NO: 2 or 4;
e. W2A and A80I in SEQ ID NO: 2 or 4;
f. K17A and R18A in SEQ ID NO: 2 or 4;
g. R10A, K17A, R18A, and R97A in SEQ ID NO: 2;
h. R10A, K17A, R18A, and K97A in SEQ ID NO: 4;
i. C9V, C28A, C78V, C204A and C206V in SEQ ID NO: 2;
j. C9V, C78V, C202A and C204V in SEQ ID NO: 4;
k. C9V, C28A, C78V, C204A, C206V and C426S in SEQ ID NO: 2;
l. E69G, D103G, and D136G in SEQ ID NO: 2 or 4;
m. E179G, D215G and D247G in SEQ ID NO: 2;
n. E179G, D213G and D245G in SEQ ID NO: 4;
o. E231G, E291G, and E332G in SEQ ID NO: 2;
p. E229G, E289G, and E330G in SEQ ID NO: 4; or
q. N61Q and N131Q in SEQ ID NO: 2 or 4.

According to some embodiments, the fragment comprises or consists of an amino acid sequence selected from SEQ ID NO: 74-85, 90-103 and 108-113.

According to some embodiments, the fragment comprises or consists of an amino acid sequence selected from SEQ ID NO: 81, 82, 90, 100 and 108.

According to some embodiments, an analog or derivative thereof comprises at least 85% identity to DSG1 or DSG3.

According to some embodiments, the fragment comprises at least 20 sequential amino acids from DSG1 or DSG3.

According to some embodiments, the fragment comprises at least one B cell receptor (BCR)-specific epitope target of autoantibodies.

According to some embodiments, the composition comprises a first or second polypeptide comprising a sequence selected from SEQ ID NO: 5-73.

According to some embodiments, the hinge domain, the CH2 domain or the CH3 domain comprises at least one mutation that reduces antibody dependent cell cytotoxicity (ADCC).

According to some embodiments, the at least one mutation is selected from:
a. a mutation of the hinge domain comprising an L19A and an L20A mutation of SEQ ID NO: 125; and
b. a mutation of the CH2 domain comprising an N59A mutation of SEQ ID NO: 139.

According to some embodiments, the composition further comprises at least one effector moiety capable of inducing cell death in a cell binding said composition.

According to some embodiments, the effector moiety is not an Fc domain.

According to some embodiments, the effector moiety is selected from: alpha-amanitin, PNU-159682, tesirine, deruxtecan (Dxd), mertansine, monomethyl auristatin E (MMAE), monomethyl auristatin F (MMAF) and a combination thereof.

According to another aspect, there is provided a pharmaceutical composition comprising a polypeptide of the invention or a composition of the invention and a pharmaceutically acceptable carrier, excipient or adjuvant.

According to another aspect, there is provided a method of treating Pemphigus vulgaris or Pemphigus foliaceus in a subject in need thereof, the method comprising administering to the subject a composition comprising a fragment of an extracellular domain of DSG1 or DSG3 or an analog or derivative thereof, thereby treating Pemphigus vulgaris or Pemphigus foliaceus.

According to some embodiments, the DSG1 lacks a prodomain and comprises or consists of SEQ ID NO: 2 and the DSG3 lacks a prodomain and comprises or consists of SEQ ID NO: 4; or the DSG1 comprises a prodomain and comprises or consist of SEQ ID NO: 1 and the DSG3 comprises a prodomain and comprises or consists of SEQ ID NO: 3.

According to some embodiments, the composition comprises a polypeptide of the invention.

According to some embodiments, the composition is a composition of the invention.

According to some embodiments, the composition is a pharmaceutical composition of the invention.

According to some embodiments, the method further comprises reducing in the subject the levels of circulating antibodies against DSG1, DSG3 or both.

According to some embodiments, the treating comprises decreasing the concentration of circulating autoantibodies against DSG1, DSG3 or both.

According to some embodiments, the composition comprises an Fc region and the treating comprises killing B cells producing anti-DSG1 or DSG3 autoantibodies.

According to some embodiments, the B cells are autoreactive B cells producing autoantibodies against a fragment of the composition.

According to another aspect, there is provided a nucleic acid molecule encoding a polypeptide of the invention.

According to another aspect, there is provided a nucleic acid system comprising a nucleic acid molecule, wherein a first nucleic acid molecule encodes the first polypeptide of a composition of the invention and a second nucleic acid molecule encodes the second polypeptide of a composition of the invention.

According to some embodiments, the nucleic acid system further comprises, a third nucleic acid molecule encoding the third polypeptide of a composition of the invention, a fourth nucleic acid molecule encoding the fourth polypeptide of a composition of the invention, or both.

According to another aspect, there is provided a method of producing a polypeptide of the invention or a composition of the invention, the method comprising expressing a nucleic acid system of the invention in a cell, wherein the nucleic acid system is configured to produce the encoded polypeptide in the cell, thereby producing a polypeptide of the invention or a composition of the invention.

According to another aspect, there is provided a method for producing a protein the method comprising:
obtaining a first fragment of an extracellular domain of DSG1 or an analog or derivative thereof or a fragment of an extracellular domain of DSG3 or an analog or derivative thereof, and a second fragment of an extracellular domain of DSG1 or an analog or derivative thereof or a fragment of an extracellular domain of DSG3 or an analog or derivative thereof, linking the first fragment to a first dimerization domain to produce a first polypeptide chain and linking the second fragment to a second dimerization domain to produce a second polypeptide chain wherein the first and second dimerization domains are capable of dimerizing with each other, and contacting the first polypeptide and the second polypeptide under conditions sufficient to induce the dimerization; or culturing a host cell comprising one or more vectors comprising a nucleic acid sequence encoding at least two polypeptide chains, wherein the two polypeptide chains are produced by:
   i. obtaining a first fragment of an extracellular domain of DSG1 or an analog or derivative thereof or a fragment of an extracellular domain of DSG3 or an analog or derivative thereof and a second fragment of an extracellular domain of DSG1 or an analog or derivative thereof or a fragment of an extracellular domain of DSG3 or an analog or derivative thereof; and
   ii. linking the first fragment to a first dimerization domain to produce a first polypeptide chain and linking the second fragment to a second dimerization domain to produce a second polypeptide chain wherein the first and second dimerization domains are capable of dimerizing with each other;
thereby producing a protein.

According to some embodiments, the protein complex is a protein complex of a composition of the invention.

According to some embodiments, the method further comprises:
a. linking a third dimerization domain to the first dimerization domain or first fragment within the first polypeptide chain; obtaining a third fragment of an extracellular domain of DSG1 or an analog or derivative thereof or a fragment of an extracellular domain of DSG3 or an analog or derivative thereof, and linking the third fragment to a fourth dimerization domain to produce a third polypeptide chain wherein the third dimerization domain and the fourth dimerization domain are capable of dimerizing to each other; and contacting the first, second, and third polypeptides under conditions sufficient to induce the dimerization; or
b. expressing in the host cell a nucleic acid sequence encoding a third polypeptide chain produced by:
   i. obtaining a third fragment of DSG1 or an analog or derivative thereof or a fragment of an extracellular domain of DSG3 or an analog or derivative thereof; and
   ii. linking the third fragment to a fourth dimerization domain to produce a third polypeptide chain;
wherein the first polypeptide chain further comprises a third dimerization domain and wherein the third dimerization domain and the fourth dimerization domain or capable of dimerizing to each other.

According to some embodiments, the method further comprises:
a. linking a sixth dimerization domain to the second dimerization domain or second fragment within the second polypeptide chain; obtaining a fourth fragment of an extracellular domain of DSG1 or an analog or derivative thereof or a fragment of an extracellular domain of DSG3 or an analog or derivative thereof, and linking the fourth fragment to a fifth dimerization domain to produce a fourth polypeptide chain wherein the fifth dimerization domain and the sixth dimerization domain are capable of dimerizing to each other; and contacting the first, second, third and fourth polypeptides under conditions sufficient to induce the dimerization; or
b. expressing in the host cell a nucleic acid sequence encoding a fourth polypeptide chain produced by:
   i. obtaining a fourth fragment of an extracellular domain of DSG1 or an analog or derivative thereof or a fragment of an extracellular domain of DSG3 or an analog or derivative thereof; and
   ii. linking the fourth fragment to a fifth dimerization domain to produce a fourth polypeptide chain;
wherein the second polypeptide chain further comprises a sixth dimerization domain and wherein the fifth dimerization domain and the sixth dimerization domain or capable of dimerizing to each other.

According to some embodiments, the method further comprises producing at least one mutation in a cadherin domain of the protein or truncating the protein to remove at least one cadherin domain or a portion thereof.

According to some embodiments, the method further comprises measuring aggregation of the mutated or truncated protein and selecting a mutated or truncated protein with reduced aggregation.

According to another aspect, there is provided a protein produced by a method of the invention.

According to another aspect, there is provided a method for producing a protein the method comprising:
a. obtaining a fragment of an extracellular domain of DSG1 or an analog or derivative thereof or a fragment of an extracellular domain of DSG3 or an analog or derivative thereof;
b. generating at least one mutation in the fragment to produce a mutated fragment;
c. measuring solubility, aggregation or both of the mutated fragment; and
d. selecting at least one mutated fragment with increased solubility, decreased aggregation or both as compared to the obtained fragment;
thereby producing a protein.

According to some embodiments, the method further comprises linking an effector moiety to at least one of the polypeptide chains or the mutated fragment, wherein the effector moiety is capable of killing a cell that binds the at least one polypeptide.

According to some embodiments, the effector moiety is not an Fc domain.

According to some embodiments, the effector moiety is selected from: alpha-amanitin, PNU-159682, tesirine, deruxtecan (Dxd), mertansine, monomethyl auristatin E (MMAE), monomethyl auristatin F (MMAF) and a combination thereof.

According to some embodiments, the effector moiety is an Fc domain comprising at least one mutation that increases ADCC or CDC.

According to some embodiments, the effector moiety is an Fc domain comprising SEQ ID NO: 60 or SEQ ID NO: 62 comprising a plurality of mutations selected from: L15V/F23L/R72P/Y80L/P176L, S19D/A110L/1112E, G16A/A110L/1112E, and G16A/S47E/H48F/S104T/1112E within the SEQ ID NO: 60 or SEQ ID NO: 62.

According to another aspect, there is provided a protein produced by a method of the invention.

According to another aspect, there is provided a method of determining suitability of a subject in need thereof to be treated by a method of the invention, the method comprising receiving a sample from the subject, contacting the sample with a protein of the invention or a composition of the invention and determining binding of autoantibodies within the sample to the protein or the composition, wherein binding of autoantibodies to the protein or the composition indicates the subject is suitable to be treated by a method of the invention, thereby determining suitability of the subject to be treated.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1F: Diagrams of five possible embodiments of the four-chain therapeutic agent of the invention: (1A) shows a general embodiment of a molecule for treating DSG1/DSG3 positive PV/PF, (1B) shows an embodiment in which the four chains each comprise a different protein fragment, (1C) shows an embodiment in which the four protein fragments are all the same, (1D) shows an embodiment in which the two heavy chains are identical and the two light chains are identical, (1E) shows an embodiment in which the two heavy chains are different and the two light chains are identical, and (1F) shows an embodiment in which the two heavy chains are contain the same protein fragment and the two light chains contain different protein fragments.

FIGS. 2A-2N: Diagrams of possible embodiments of the two-chain therapeutic agent of the invention: (2A) shows general embodiments of a molecule with two heavy chains for treating DSG1/DSG3 positive PV/PF, (2B) shows embodiments in which at least one of the CH1, CH2 or CH3 domains has been excluded, (2C) shows an embodiment in which the two protein fragments are the same, (2D) shows an embodiment in which the two protein fragments are different, (2E) shows an embodiment in which the two protein fragments are different and the molecule does not contain a CH1 domain, (2F) shows an embodiment in which the two protein fragments are different and the molecule does not contain a CH1 domain or a hinge domain, (2G) shows a general embodiment in which two tandem fragments are included in each heavy chain and connected via a linker, (2H) shows the tandem fragment configuration in which all the subunits are the same, (2I) shows the tandem fragment configuration in which all the subunits are the same without the CH1 domain, (2J) shows the tandem fragment configuration in which each heavy chain contains the same two different fragments, (2K) shows the tandem fragment configuration in which each heavy chain contains the same two different fragments without a CH1 domain, (2L) shows the tandem fragment configuration in which the two have chains contain different fragments that are not the same, (2M) shows the tandem fragment configuration in which the two have chains contain different fragments that are not the same without the CH3 domain, and (2N) shows a general embodiment of a molecule with one heavy chain and one light chain.

FIGS. 3A-3D: Diagrams of four possible embodiments of the three-chain therapeutic agent of the invention: (3A) shows a general embodiment of a molecule with two heavy chains and one light chain, (3B) shows an embodiment in which the three protein fragments are the same, (3C) shows an embodiment in which each of the protein fragments are different, (3D) shows an embodiment in which two of the protein fragments are the same and the third is different.

FIG. 4: Diagram of an embodiment of a four-chain therapeutic agent for treating DSG1/DSG3 positive PV/PF of the invention similar to those shown in FIG. 1 but in which the four chains each comprise a different protein fragment and a different immunoglobulin scaffold which promotes formation of the four-chain molecule.

FIGS. 5A-5B: (5A-5B) Diagrams of generic embodiments of the four-chain therapeutic agent of the invention: (5A) shows a generic embodiment of four chains in which two contain chains contain two dimerization domain and two chains contain a single dimerization domain, and (5B) shows an embodiment with optional linkers separating the various domains and fragments.

FIGS. 6A-6G: Diagrams of single chain therapeutic agents of the invention: (6A) shows an embodiment of a single chain molecule containing fragments from two different DSG1/3 fragments, (6B) shows embodiments of fragments comprising a truncation of DSG1 or DSG3, (6C) shows an embodiments of a single chain molecule containing three different fragments, (6D) shows an embodiments of a single chain molecule containing four different fragments, (6E) shows an embodiments of a single chain molecule containing fragments from two different DSG proteins/domains and a heavy chain constant region, (6F) shows an embodiments of a single chain molecule containing fragments from two different DSG proteins/domains and a CH3-CH2 fragment of the heavy chain constant region, and (6G) shows the single chain molecules of 6A and 6C-6F with amino acid (AA) linkers separating various domains.

FIGS. 7A-7F: (7A) Photographs of an SDS-PAGE gel (left) and a Western blot (right), both in reducing conditions showing molecules 290 and 292. (7B) Photographs of SDS-PAGE gels in reducing conditions showing molecules 291, 293, 294, 295 and 296. (7C) Line graphs of depletion rates of anti-DSG3 IgG in the presence of increasing concentration of the DSG3 containing molecules of the invention for 4 different human PV patients' sera. (7D) Line graph of the average of the 4 human PV patients' sera from 7C. (7E) Bar graph of the average depletion rates of anti-DSG3 IgG in the presence of 3.85 μM of the molecules of the invention for 6 different human PV patients' sera. (7F) Bar graph of the average depletion rates of anti-DSG1 IgG in the presence of 0.77 μM of molecules of the invention for 1 human Pemphigus patients' serum.

FIGS. 8A-8E. (8A, 8C, 8E) Photographs of an SDS-PAGE gels in non-reducing conditions showing molecules (8A) CRD-291, CRD-657 and CRD-662; (8C) CRD-293, CRD-674 and CRD-679; and (8E) CRD-294 and CRD-685. (8B, 8D) Bar graphs of percent monomer purity as determined by HPLC for (8B) CRD-291, CRD-657 and CRD-700 and (8D) CRD-293, CRD-674 and CRD-701.

FIGS. 9A-9F. (9A, 9F) Bar graph of average depletion rates of anti-DSG1 IgG in the presence of (9A) 1.54 μM of molecules of the invention comprising the full extracellular domain of DSG1 and (9F) 3.85 μM of molecules of the invention comprising truncations of the extracellular domain of DSG1. (9B-9E) Bar graph of average depletion rates of anti-DSG3 IgG in the presence of (9B) 1.54 μM of molecules of the invention comprising the full extracellular domain of DSG3 and (9C-E) 3.85 μM of molecules of the invention comprising truncations of the extracellular domain of DSG3. FIG. 9C depicts the average depletion in 8 different sera samples for all the tested molecules except CRD-291. CRD-291 was tested only in one sample and the direct comparison in that sera sample with CRD-657 in that sera sample is provided in FIG. 9D.

FIGS. 10A-C. (10A) Bar chart of the ratio between the MFI of specific versus background staining of various molecules of the invention to two different hybridomas. 5H10 is a DSG3 specific hybridoma and g-66 is a hybridoma specific to an irrelevant protein. (10B) Bar chart of relative binding to hybridoma 5H10 of various molecules of the invention. Binding is standardized relative to binding of CRD-924. (10C) Line graph of binding of CRD-691 to DSG3 specific B cell hybridoma 5H10 and non-specific hybridoma a-18-C5-F6. Hybridomas were incubated in the presence of 32 nM, 10.6 nM or 3.5 nM of CRD-691. The Y-axis represents the MFI value of each sample, divided by the BCR MFI value of each hybridoma. The 5H10 hybridoma was stained with PE-anti-MOUSE BCR and the a-18-C5-F6 hybridoma was stained with anti-RAT BCR, to assess BCR expression.

FIGS. 11A-C. (11A) Bar graph of average depletion rates of anti-DSG3 IgG in the presence of 3.85 μM or 0.769 μM of unconjugated or tesirine-conjugated CRD-924. (11B) Bar graph of 5H10 hybridoma cell binding by various concentrations of unconjugated or tesirine-conjugated CRD-924. The Y-axis represents the MFI value of each sample, divided by the BCR MFI value. (11C) Line graph of the percent increase in cell killing by CRD-924-tesirine over the unconjugated molecule. CRD-924-tesirine's killing was specific to the DSG-3 hybridoma and tesirine background killing, of an irrelevant hybridoma (cocultured with the target hybridoma) was minimal and non-dose dependent.

### DETAILED DESCRIPTION OF THE INVENTION

The present invention, in some embodiments, provides compositions comprising a fragment of a first human receptor target of Pemphigus vulgaris (PV) and Pemphigus foliaceus (PF) autoantibodies or an analog or derivative thereof and a fragment of a second human protein receptor target of Pemphigus vulgaris and Pemphigus foliaceus autoantibodies or an analog or derivative thereof. Compositions further comprising an effector moiety that is not an unmodified Fc domain are also provided. Protein complexes comprising at least two polypeptide chains wherein a first chain comprises a fragment of a first human protein target of Pemphigus vulgaris and Pemphigus foliaceus autoantibodies or an analog or derivative thereof and a first dimerization domain and a second chain comprises a fragment of a second human protein target of Pemphigus vulgaris and Pemphigus foliaceus autoantibodies or an analog or derivative thereof and a second dimerization domain capable of dimerizing with the first dimerization domain are also provided. As are protein complexes in which one of the chains comprises an effector moiety that is not an unmodified Fc domain Polypeptides comprising fragments of an extracellular domain of desmoglein-1 (DSG1) or desmoglein-3 (DSG3) comprising at least one mutation that increases solubility, decreases aggregation or both or which is devoid of extracellular domains 3 and 4 are also provided. As are polypeptides comprising an effector moiety that is not an unmodified Fc domain The present invention further concerns pharmaceutical composition comprising the compositions and/or protein complexes, nucleic acids encoding the polypeptides of the compositions and/or protein complexes and methods of treatment and determining suitability for treatment using the compositions and/or protein complexes; as well as methods of producing the compositions and/or protein complexes.

By a first aspect, there is provided a composition comprising a fragment of a first protein target of PV or PF autoantibodies or an analog or derivative thereof.

By another aspect, there is provided a composition comprising a fragment of a first protein target of PV or PF autoantibodies and a fragment of a second protein target of PV or PF autoantibodies or an analog or derivative thereof.

By another aspect, there is provided a protein comprising a fragment of a first protein target of PV or PF autoantibodies or an analog or derivative thereof.

By another aspect, there is provided a protein comprising a fragment of a first protein target of PV or PF autoantibodies or an analog or derivative thereof and a fragment of a second protein target of PV or PF autoantibodies or an analog or derivative thereof.

By another aspect, there is provided a protein complex comprising at least two polypeptide chains, wherein a first polypeptide chain comprises a fragment of a first protein target of PV or PF autoantibodies or an analog or derivative thereof and a first dimerization domain and a second polypeptide chain comprising a fragment of a second protein target of PV or PF autoantibodies or an analog or derivative thereof and second dimerization domain.

In some embodiments, the composition comprises a protein complex comprising at least two polypeptide chains, wherein a first polypeptide chain comprises a fragment of a first protein target of PV or PF autoantibodies or an analog or derivative thereof and a first dimerization domain and a second polypeptide chain comprising a fragment of a second protein target of PV or PF autoantibodies or an analog or derivative thereof and second dimerization domain. In some embodiments, the composition comprises a protein complex of the invention. In some embodiments, the composition comprises a protein of the invention. In some embodiments, the protein is a recombinant protein. In some embodiments, the protein is a fusion protein.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the protein complex is an immunoglobulin (Ig)-like complex. In some embodiments, the protein complex comprises an Ig-like scaffold. In some embodiments, the protein complex comprises an Ig-like backbone. In some embodiments, the protein complex is an Ig Fc-fusion complex. In some embodiments, the composition is devoid of an antibody variable domain. In some embodiments, the protein complex is devoid of an antibody variable domain. In some embodiments, the composition is devoid of a variable domain. In some embodiments, the protein complex is devoid of a variable domain. In some embodiments, the first chain is devoid of a variable domain. In some embodiments, the second chain is devoid of a variable domain. In some embodiments, the protein complex is a multi-chain complex. In some embodiments, the composition is a therapeutic composition. In some embodiments, the protein complex is a therapeutic complex. In some embodiments, the composition is for use in a therapeutic method. In some embodiments, the protein complex is for use in a therapeutic method. In some embodiments, the composition is for use in production of a medicament. In some embodiments, the protein complex is for use in the production of a medicament. In some embodiments, the composition is for use in treating PV. In some embodiments, the composition is for use in treating PF. In some embodiments, the protein complex is for use in treating PV. In some embodiments, the protein complex is for use in treating PF. In some embodiments, the protein complex is for use in diagnosing PV. In some embodiments, the protein complex is for use in diagnosing PF. In some embodiments, the protein complex is for use in determining appropriate treatment in PV. In some embodiments, the protein complex is for use in determining appropriate treatment in PF. In some embodiments, the protein complex is for use in characterizing the serological response in PV. In some embodiments, the protein complex is for use in characterizing the serological response in PF. In some embodiments, the protein complex is for use in determining the autoantibody titer in PV. In some embodiments, the protein complex is for use in determining the autoantibody titer in PF.

As used herein, the term “polypeptide chain” refers to a polymer of amino acids linked by peptide bonds from an amino terminus (N-terminus) to a carboxyl terminus (C-terminus). In some embodiments, the polypeptide chain is a recombinant polypeptide. In some embodiments, a polypeptide chain comprises at least 5, 10, 15, 20, 25, 30, 35, 40, 45, 50, 55, 60, 65, 70, 75, 80, 85, 90, 100, 150, 200, 250, 300, 350, 400, 450 or 500 amino acids. Each possibility represents a separate embodiment of the invention. In some embodiments, a polypeptide chain comprises at most 300, 350, 400, 450, 500, 550, 600, 650, 700, 750, 800, 850, 900, 950, 1000, 1250, 1500, 1750, 2000, 2250, 2500, 2750, 3000, 3250, 3500, 3750, 4000, 4250, 4500, 4750, or 5000 amino acids. Each possibility represents a separate embodiment of the invention.

As used herein, the term “recombinant polypeptide” refers to a protein which is coded for by a recombinant DNA and is thus not naturally occurring. In some embodiments, the protein complex is not naturally occurring. In some embodiments, the polypeptide chain is not naturally occurring. In some embodiments, the recombinant polypeptide is a synthetic polypeptide. The term “recombinant DNA” refers to DNA molecules formed by laboratory methods. Generally, this recombinant DNA is in the form of a vector, plasmid or virus used to express the recombinant protein in a cell. Production of recombinant proteins by cellular expression is well known in the art and any method of recombinant protein expression may be used to produce the polypeptide of the invention. Cell free expression systems for recombinant protein production may also be employed.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, a nucleic acid molecule of the invention is expressed in a cell to produce a polypeptide of the invention. In some embodiments, a nucleic acid complex of the invention is expressed in a cell to produce a protein complex of the invention. In some embodiments, the RNA is a vector.

Expressing a DNA sequence or an RNA within a cell is well known to one skilled in the art. It can be carried out by, among many methods, transfection, viral infection, or direct alteration of the cell's genome. In some embodiments, the DNA sequence is in an expression vector such as plasmid or viral vector. In some embodiments, a Kozak sequence is inserted upper stream of the transcription initiating codon. In some embodiments, the Kozak sequence enhances the amount of protein expresses.

In some embodiments, the protein complex comprises at least two polypeptide chains. In some embodiments, the protein complex comprises at least three polypeptide chains. In some embodiments, the protein complex comprises at least four polypeptide chains. In some embodiments, the protein complex comprises or consists of two polypeptide chains. In some embodiments, the protein complex comprises or consists of three polypeptide chains. In some embodiments, the protein complex comprises or consists of four chains. In some embodiments, the polypeptide chains are the same. In some embodiments, the polypeptide chains are different. In some embodiments, at least two of the polypeptide chains are the same. In some embodiments, at least two of the polypeptide chains are different.

### Proteins

In some embodiments, the protein is a mammalian protein. In some embodiments, the mammal is a human. In some embodiments, the protein is a transmembrane protein. In some embodiments, the protein is a cell surface protein. In some embodiments, the protein is a receptor. In some embodiments, the protein is a subunit in a receptor. In some embodiments, the protein is a cell surface protein. In some embodiments, the cell surface protein is an integral membrane protein. In some embodiments, the cell surface protein is a plasma membrane embedded protein. In some embodiments, the cell surface protein is a membrane anchored protein. In some embodiments, the protein is a PV or PF-associated protein. In some embodiments, the protein is a synthetic protein. In some embodiments, the protein is a naturally occurring protein. In some embodiments, the protein is a target of PV or PF autoantibodies. In some embodiments, the protein is selected from Desmoglein 1 (DSG1) and Desmoglein 3 (DSG3). In some embodiments, the protein is DSG1. In some embodiments, the protein is DSG3.

As used herein, the term “receptor” refers to a protein expressed on the surface of a cell that is capable of binding a ligand. In some embodiments, a receptor is a protein capable of transducing a signal to the cytoplasm of the cell. In some embodiments, a receptor comprises a ligand binding domain. In some embodiments, a receptor comprises a transmembrane domain. In some embodiments, a receptor comprises an intracellular domain.

In some embodiments, the fragment comprises an extracellular domain (ECD) of the protein. In some embodiments, the fragment comprises a fragment of an extracellular domain of the protein. In some embodiments, the fragment consists of the extracellular domain of a fragment thereof. In some embodiments, the fragment consists of an extracellular domain of the protein. In some embodiments, the fragment consists of a fragment of an extracellular domain of the protein. In some embodiments, the fragment comprises a transmembrane domain of the protein. In some embodiments, the fragment is devoid of a transmembrane domain of the protein. In some embodiments, the fragment is devoid of an intracellular domain of the protein. In some embodiments, the chain is devoid of a transmembrane domain. In some embodiments, the chain is devoid of an intracellular domain. In some embodiments, the fragment includes a sequence from a homologous human protein. In some embodiments, the fragment includes a sequence from a homologous non-human protein. In some embodiments, the fragment includes mutations in the human protein.

In some embodiments, the fragment comprises at least 5 amino acids of the protein. In some embodiments, the fragment comprises at least 10 amino acids of the protein. In some embodiments, the fragment comprises at least 5, 10 15, 20, 25, 30, 35, 40, 45, 50, 55, 60, 65, 70, 75, 80, 85, 90, 95 or 100 amino acids. Each possibility represents a separate embodiment of the invention. In some embodiments, amino acids of the protein are consecutive amino acids of the protein. In some embodiments, the fragment comprises less than 100% of the protein. In some embodiments, the fragment comprises less than 100% of an extracellular domain of the protein. In some embodiments, the fragment comprises less than 100, 99, 97, 95, 90, 85, 80, 75, 70, 65, 60, 55 or 50% of the protein. Each possibility represents a separate embodiment of the invention. In some embodiments, the fragment comprises less than 100, 99, 97, 95, 90, 85, 80, 75, 70, 65, 60, 55 or 50% of an extracellular domain of the protein. Each possibility represents a separate embodiment of the invention. In some embodiments, the fragment comprises between 5-500, 5-250, 5-100, 5-50, 10-500, 10-250, 10-100, 10-50, 20-500, 20-250, 20-200, 20-50, 25-500, 25-250, 25-100, 25-50, 50-500, 50-250, 50-100, 100-500, or 100-250 amino acids. Each possibility represents a separate embodiment of the invention. In some embodiments, a fragment comprises at most 20, 30, 40, 50, 60, 70, 75, 80, 90, 100, 110, 120, 125, 130, 140, 150, 160, 170, 175, 180, 190, 200, 210, 220, 225, 230, 240, 250, 260, 270, 275, 280, 290, 300, 310, 320, 325, 330, 340, 350, 360, 370, 375, 380, 390, 400, 410, 420, 425, 430, 440, 450, 460, 470, 475, 480, 490, 500, 550, 600, 650, 700, 750, 800, 850, 900, 950 or 1000 amino acids. Each possibility represents a separate embodiment of the invention.

The extracellular regions of DSG1 and DSG3 contain four extracellular domains (ECs) each. These are labeled EC1, EC2, EC3 and EC4 from the N-terminus to the C-terminus of the extracellular region. In some embodiments, the EC1 of DSG1 comprises or consists of EWIKFAAACREGEDNSKRNPIAKIHSDCAANQQVTYRISGVGIDQPPYGIFVINQKT GEINITSIVDREVTPFFIIYCRALNSMGQDLERPLELRVRVLDINDNPPVFS (SEQ ID NO: 114). In some embodiments, the EC2 of DSG1 comprises or consists of MATFAGQIEENSNANTLVMILNATDADEPNNLNSKIAFKIIR QEPSDSPMFIINRNT GEIRTMNNFLDREQYGQYALAVRGSDRDGGADGMSAECECNIKILDVNDNIPYM E (SEQ ID NO: 115). In some embodiments, the EC3 of DSG1 comprises or consists of QSSYTIEIQENTLNSNLLEIR VIDLDEEFSANWMA VIFFISGNEGNWFEIEMNERTNV GILKVVKPLDYEAMQSLQLSIGVRNKAEFHHSIMSQYKLKASAISVTVLNVIEGPV F (SEQ ID NO: 116). In some embodiments, the EC4 of DSG1 comprises or consists of RPGSKTYVVTGNMGSNDKVGDFVATDLDTGRPSTTVRYVMGNNPADLLAVDSR TGKLTLKNKVTKEQYNMLGGKYQGTILSIDDNLQRTCTGTININIQSFGNDDRTNT EPN (SEQ ID NO: 117). In some embodiments, the EC1 of DSG3 comprises or consists of EWVKFAKPCREGEDNSKRNPIAKITSDYQATQKITYRISGVGIDQPPFGIFVVDKNT GDINITAIVDREETPSFLITCRALNAQGLDVEKPLILTVKILDINDNPPVFS (SEQ ID NO: 118). In some embodiments, the EC2 of DSG3 comprises or consists of QQIFMGEIEENSASNSLVMILNATDADEPNHLNSKIAFKIVSQEPAGTPMFLLSRNT GEVRTLTNSLDREQASSYRLVVSGADKDGEGLSTQCECNIKVKDVNDNFPMFR (SEQ ID NO: 119). In some embodiments, the EC3 of DSG3 comprises or consists of DSQYSARIEENILSSELLRFQVTDLDEEYTDNWLAVYFFTSGNEGNWFEIQTDPRT NEGILKVVKALDYEQLQSVKLSIAVKNKAEFHQSVISRYRVQSTPVTIQVINVREGI AF (SEQ ID NO: 120). In some embodiments, the EC4 of DSG3 comprises or consists of RPASKTFTVQKGISSKKLVDYILGTYQAIDEDTNKAASNVKYVMGRNDGGYLMID SKTAEIKFVKNMNRDSTFIVNKTITAEVLAIDEYTGKTSTGTVYVRVPDFNDNCPT AVLEK (SEQ ID NO: 121).

In some embodiments, the fragment comprises EC1 of DSG1. In some embodiments, the fragment comprises EC2 of DSG1. In some embodiments, the fragment comprises EC3 of DSG1. In some embodiments, the fragment comprises EC4 of DSG1. In some embodiments, the fragment comprises EC1 of DSG3. In some embodiments, the fragment comprises EC2 of DSG3. In some embodiments, the fragment comprises EC3 of DSG3. In some embodiments, the fragment comprises EC4 of DSG3. In some embodiments, the fragment comprises or consists of EC1 and EC2 of DSG1. In some embodiments, the fragment comprises or consists of EC1 and EC2 of DSG3. In some embodiments, the fragment is devoid of EC3 of DSG1. In some embodiments, the fragment is devoid of EC3 of DSG3. In some embodiments, the fragment is devoid of EC4 of DSG1. In some embodiments, the fragment is devoid of EC4 of DSG3. In some embodiments, the fragment is devoid of EC3 and EC4 of DSG1. In some embodiments, the fragment is devoid of EC3 and EC4 of DSG3. In some embodiments, the fragment comprises an amino acid sequence selected from SEQ ID NO: 87, 90-91, 105 and 108-109. In some embodiments, the fragment consists of an amino acid sequence selected from SEQ ID NO: 87, 90-91, 105 and 108-109. In some embodiments, the fragment is from DSG1 and comprises or consists of a sequence selected from SEQ ID NO: 87 and 90-91. In some embodiments, the fragment is from DSG3 and comprises or consists of a sequence selected from SEQ ID NO: 105 and 108-109. It will be understood that each sequence represents a separate embodiment of the invention.

In some embodiments, the chain comprises at least one fragment. In some embodiments, the chain comprises at least two fragments. In some embodiments, the fragments are separated by a linker. In some embodiments, the linker is a flexible linker. In some embodiments, the linker comprises increased solubility as compared to a region of the protein excluded from the chain. In some embodiments, a region of the protein is replaced by a region of protein that is not the protein. In some embodiments, the replacement region comprises increased solubility as compared to the region of the protein that has been replaced. In some embodiments, the replacement region comprises increased protein stability as compared to the region of the protein that has been replaced.

In some embodiments, the protein is a target of antibodies. As used herein, the term “antibody” includes all classes of IgA, IgD, IgE, IgG and IgM and also includes all subclasses thereof. In some embodiments, the antibody is a circulating antibody. In some embodiments, the antibody is a naturally occurring antibody. In some embodiments, the antibodies are autoantibodies.

As used herein, the term “autoantibodies” refers to antibodies generated by a subject's own immune system against at least one of the subject's own proteins. In some embodiments, an autoantibody is an autoreactive antibody. In some embodiments, autoantibodies target self-antigens. Self-antigens are also known as autoantigens. In some embodiments, the autoantibodies are associated with PV or PF. In some embodiments, the autoantibodies characterize PV or PF. In some embodiments, the autoantibodies are autoantibodies of PV or PF. In some embodiments, autoantibodies are generated by auto-reactive B cells. In some embodiments, the protein is an antigen of the antibodies. In some embodiments, the fragment comprises an antigen of the antibodies. In some embodiments, the fragment comprises at least one antigen of the antibodies. In some embodiments, the fragment comprises at least two antigens of the antibodies. In some embodiments, the fragment comprises at least 1, 2, 3, 4, 5, 6, 7, 8, 9 or 10 antigens of the antibodies. Each possibility represents a separate embodiment of the invention. In some embodiments, an antigen of the antibodies is an autoantigen. In some embodiments, the antigen is an epitope. In some embodiments, the antigen includes at least one epitope. In some embodiments, an epitope comprises at least 5 amino acids. In some embodiments, an epitope comprises 5-6 amino acids. In some embodiments, an epitope comprises 5-10 amino acids. In some embodiments, an epitope is a simple epitope. In some embodiments, a simple epitope is a linear epitope. In some embodiments, an epitope is a complex epitope. In some embodiments, a complex epitope is a 3D epitope. In some embodiments, a complex epitope is a discontinuous epitope. In some embodiments, a discontinuous epitope comprises at least two discontinuous sections of amino acids that combine to form an epitope. In some embodiments, a linker sequence is between the two sections of the epitope.

As used herein, the term “analog” includes any peptide having an amino acid sequence substantially identical to the sequence of the protein but in which one or more residues have been conservatively substituted with a functionally similar residue. In some embodiments, an analog displays similar functionality to the original protein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. Each possibility represents a separate embodiment of the present invention. In some embodiments, the substitution is outside of an antigenic region of the protein. In some embodiments, the substitution is outside an epitope of the antibodies. In some embodiments, the analog is still a target of the antibodies. In some embodiments, the analog retains binding of autoantibodies. An analog may have deletions or mutations that result in an amino acids sequence that is different than the canonical amino acid sequence of protein. Further, an analog may be analogous to a fragment of the protein, however, in such a case the fragment must comprise at least 50 consecutive amino acids of protein or at least one epitope of the antibodies. In some embodiments, an analog is an analog to the canonical sequence of the protein.

In some embodiments, an analog to the protein comprises an amino acid sequence with at least 60%, at least 65%, at least 70%, at least 75%, at least 80%, at least 85%, at least 90%, at least 95%, at least 99% homology to the canonical amino acid sequence of the protein. Each possibility represents a separate embodiment of the invention. In some embodiments, an analog to the protein comprises an amino acid sequence with at least 60%, at least 65%, at least 70%, at least 75%, at least 80%, at least 85%, at least 90%, at least 95%, at least 99% identity to the canonical amino acid sequence of the protein. Each possibility represents a separate embodiment of the invention. In some embodiments, an analog to the protein comprises an amino acid sequence with at least 85% identity to the canonical amino acid sequence of the protein. In some embodiments, the analog is still able to bind PV or PF autoantibodies. In some embodiments, the analog is still able to sequester PV or PF autoantibodies. In some embodiments, the analog is still able to treat PV or PF. In some embodiments, the analog comprises at least one substitution. In some embodiments, an analog comprises at least 1, 2, 3, 4, 5, 6, 7, 8, 9, or 10 substitutions. Each possibility represents a separate embodiment of the invention. In some embodiments, substitution is a mutation of the canonical sequence.

The term “derivative” as used herein, refers to any polypeptide that is based off the protein and still comprises retains binding of the antibodies. A derivative is not merely a fragment of the protein, nor does it have amino acids replaced or removed (an analog), rather it may have additional modification made to the protein, such as post-translational modification. Further, a derivative may be a derivative of a fragment of the protein, however, in such a case the fragment must comprise at least 50 consecutive amino acids of the protein or at least one epitope of the antibodies. In some embodiments, the derivative is a derivative of a canonical sequence of the protein.

In some embodiments, a derivative to the protein comprises an amino acid sequence with at least 60%, at least 65%, at least 70%, at least 75%, at least 80%, at least 85%, at least 90%, at least 95%, at least 99% homology to the canonical amino acid sequence of the protein. Each possibility represents a separate embodiment of the invention. In some embodiments, a derivative to the protein comprises an amino acid sequence with at least 60%, at least 65%, at least 70%, at least 75%, at least 80%, at least 85%, at least 90%, at least 95%, at least 99% identity to the canonical amino acid sequence of the protein. Each possibility represents a separate embodiment of the invention. In some embodiments, a derivative to the protein comprises an amino acid sequence with at least 85% identity to the canonical amino acid sequence of the protein. In some embodiments, the derivative is still able to bind PV or PF autoantibodies. In some embodiments, the derivative is still able to sequester PV or PF autoantibodies. In some embodiments, the derivative is still able to treat PV or PF. In some embodiments, a derivative is the protein or fragment with a mutation.

Canonical amino acid sequences of known proteins are well known in the art. They can be found in a variety of databases, including UniProt, NCBI, and the UCSC Genome Browser. Any sequence accepted as a canonical sequence may be employed. For a non-limiting example, human DSG1 is encoded by the DSG1 gene, its canonical nucleic acid sequence can be found in Entrez gene 1828, its canonical protein coding mRNA sequence can be found in NM_001942, its canonical amino acid sequence can be found in NP_001933 and UniProt number Q02413. Similarly, human DSG3 is encoded by the DSG3 gene, its canonical nucleic acid sequence can be found in Entrez gene 1830, its canonical protein coding mRNA sequence can be found in NM_001944, its canonical amino acid sequence can be found in NP_001935 and UniProt number P32926. In some embodiments, a canonical sequence is a sequence identical to the sequence present in at least 50, 60, 70, 75, 80, 90, 95, 97, or 99 percent of a population. Each possibility represents a separate embodiment of the invention. In some embodiments, a canonical sequence is a sequence identical to the most prevalent sequence present in a population. In some embodiments, the population is a disease population. In some embodiments, the population is a population with the autoimmune disease.

In some embodiments, a canonical amino acid sequence of an extracellular domain of or DSG1 comprises consists of EFRIQVRDYNTKNGTIKWHSIRRQKREWIKFAAACREGEDNSKRNPIAKIHSDCAA NQQVTYRISGVGIDQPPYGIFVINQKTGEINITSIVDREVTPFFIIYCRALNSMGQDLE RPLELRVRVLDINDNPPVFSMATFAGQIEENSNANTLVMILNATDADEPNNLNSKI AFKIIRQEPSDSPMFIINRNTGEIRTMNNFLDREQYGQYALAVRGSDRDGGADGMS AECECNIKILDVNDNIPYMEQSSYTIEIQENTLNSNLLEIR VIDLDEEFSANWMAVIF FISGNEGNWFEIEMNERTNVGILKVVKPLDYEAMQSLQLSIGVRNKAEFHHSIMSQ YKLKASAISVTVLNVIEGPVFRPGSKTYVVTGNMGSNDKVGDFVATDLDTGRPST TVRYVMGNNPADLLAVDSRTGKLTLKNKVTKEQYNMLGGKYQGTILSIDDNLQR TCTGTININIQSFGNDDRTNTEPN (SEQ ID NO: 1). In some embodiments, DSG1 lacks its prodomain. In some embodiments, the prodomain is an N-terminal prodomain. In some embodiments, the prodomain consists of amino acids 1-26 of SEQ ID NO: 1. In some embodiments, a canonical amino acid sequence of DSG1 lacks a prodomain and comprises or consists of EWIKFAAACREGEDNSKRNPIAKIHSDCAANQQVTYRISGVGIDQPPYGIFVINQKT GEINITSIVDREVTPFFIIYCRALNSMGQDLERPLELRVRVLDINDNPPVFSMATFAG QIEENSNANTLVMILNATDADEPNNLNSKIAFKIIRQEPSDSPMFIINRNTGEIRTMN NFLDREQYGQYALAVRGSDRDGGADGMSAECECNIKILDVNDNIPYMEQSSYTIEI QENTLNSNLLEIRVIDLDEEFSANWMAVIFFISGNEGNWFEIEMNERTNVGILKVV KPLDYEAMQSLQLSIGVRNKAEFHHSIMSQYKLKASAISVTVLNVIEGPVFRPGSK TYVVTGNMGSNDKVGDFVATDLDTGRPSTTVRYVMGNNPADLLAVDSRTGKLT LKNKVTKEQYNMLGGKYQGTILSIDDNLQRTCTGTININIQSFGNDDRTNTEPN (SEQ ID NO: 2). In some embodiments, the extracellular domain is devoid of a signal peptide. In some embodiments, the extracellular domain further comprises a signal peptide. In some embodiments, the DSG1 signal peptide comprises or consists of MDWSFFRVVAMLFIFLVVVEVNS (SEQ ID NO: 158).

In some embodiments, a canonical amino acid sequence of an extracellular domain of DSG3 comprises or consists of ELRIETKGQYDEEEMTMQQAKRRQKREWVKFAKPCREGEDNSKRNPIAKITSDYQ ATQKITYRISGVGIDQPPFGIFVVDKNTGDINITAIVDREETPSFLITCRALNAQGLD VEKPLILTVKILDINDNPPVFSQQIFMGEIEENSASNSLVMILNATDADEPNHLNSKI AFKIVSQEPAGTPMFLLSRNTGEVRTLTNSLDREQASSYRLVVSGADKDGEGLSTQ CECNIKVKDVNDNFPMFRDSQYSARIEENILSSELLRFQVTDLDEEYTDNWLAVYF FTSGNEGNWFEIQTDPRTNEGILKVVKALDYEQLQSVKLSIAVKNKAEFHQSVISR YRVQSTPVTIQVINVREGIAFRPASKTFTVQKGISSKKLVDYILGTYQAIDEDTNKA ASNVKYVMGRNDGGYLMIDSKTAEIKFVKNMNRDSTFIVNKTITAEVLAIDEYTG KTSTGTVYVRVPDFNDNCPTAVLEK (SEQ ID NO: 3). In some embodiments, DSG3 lacks its prodomain. In some embodiments, the prodomain is an N-terminal prodomain. In some embodiments, the prodomain consists of amino acids 1-26 of SEQ ID NO: 3. In some embodiments, a canonical amino acid sequence of DSG3 lacks a prodomain and comprises or consists of EWVKFAKPCREGEDNSKRNPIAKITSDYQATQKITYRISGVGIDQPPFGIFVVDKNT GDINITAIVDREETPSFLITCRALNAQGLDVEKPLILTVKILDINDNPPVFSQQIFMGE IEENSASNSLVMILNATDADEPNHLNSKIAFKIVSQEPAGTPMFLLSRNTGEVRTLT NSLDREQASSYRLVVSGADKDGEGLSTQCECNIKVKDVNDNFPMERDSQYSARIE ENILSSELLRFQVTDLDEEYTDNWLAVYFFTSGNEGNWFEIQTDPRTNEGILKVVK ALDYEQLQSVKLSIAVKNKAEFHQSVISRYRVQSTPVTIQVINVREGIAFRPASKTF TVQKGISSKKLVDYILGTYQAIDEDTNKAASNVKYVMGRNDGGYLMIDSKTAEIK FVKNMNRDSTFIVNKTITAEVLAIDEYTGKTSTGTVYVRVPDENDNCPTAVLEK (SEQ ID NO: 4). In some embodiments, the extracellular domain is devoid of a signal peptide. In some embodiments, the extracellular domain further comprises a signal peptide. In some embodiments, the DSG3 signal peptide comprises or consists of MMGLFPRTTGALAIFVVVILVHG (SEQ ID NO: 159). In some embodiments, the DSG1 or the DSG3 signal peptide is used.

In some embodiments, the signal peptide is a signal peptide of an antibody chain. In some embodiments, the single peptide is of an antibody heavy chain. In some embodiments, the signal peptide is of an antibody light chain. In some embodiments, the signal peptide is of the Kappa light chain. In some embodiments, the signal peptide is of the Lambda light chain. In some embodiments, the heavy chain signal peptide comprises MEWSWVFLFFLSVTTGVHS (SEQ ID NO: 122). In some embodiments, the heavy chain signal peptide consists of SEQ ID NO: 122. In some embodiments, the light chain signal peptide comprises MSVPTQVLGLLLLWLTDARC (SEQ ID NO: 123). In some embodiments, the light chain signal peptide consists of SEQ ID NO: 123. In some embodiments, the signal peptide comprises of consists of MEFGLSWLFLVAILKGVQC (SEQ ID NO: 160). In some embodiments, the signal peptide comprises or consists of MGWSCIILFLVATATGVHS (SEQ ID NO: 161).

In some embodiments, the first protein and the second protein are the same protein. In some embodiments, the first and second proteins are the same proteins, and the fragments are different fragments. In some embodiments, the fragments are different fragments. In some embodiments, the fragments comprise or consist of different sequences. In some embodiments, the first and second proteins are different proteins.

In some embodiments, the protein or fragment comprises a mutation that decreases aggregation. In some embodiments, aggregation is aggregation of the protein. In some embodiments, aggregation is aggregation of the fragment. In some embodiments, aggregation is aggregation of the extracellular domain. In some embodiments, aggregation is aggregation of the complex. In some embodiments, aggregation is aggregation of the polypeptide. In some embodiments, decreasing aggregation comprises increasing solubility. In some embodiments, decreasing aggregation comprises increasing stability. In some embodiments, the protein or fragment comprises a mutation that increases solubility. In some embodiments, the protein or fragment comprises a mutation that increases stability of the protein or fragment. In some embodiments, the protein is the polypeptide. In some embodiments, the mutation is an insertion. In some embodiments, the protein is a surface protein and comprises a mutation that increases solubility. In some embodiments, the fragment is an extracellular domain of a surface protein and comprises an insertion that increases solubility.

In some embodiments, the mutation is a point mutation. In some embodiments, the mutation is selected from mutation of at least one amino acid selected from: W28, D53, E115, A106, K43, R44, R36, R123, C35, C54, C104, C230, C232, C452, E95, D129, D162, E205, D241, D273, E257, E317, E358, N87 and N157 within SEQ ID NO: 1. In some embodiments, the mutation is selected from mutation of at least one amino acid selected from: W2, D27, E89, A80, K17, R18, R10, R97, C9, C28, C78, C204, C206, C426, E69, D103, D136, E179, D215, D247, E231, E291, E332, N61 and N131 within SEQ ID NO: 2. In some embodiments, the mutation is selected from mutation of at least one amino acid selected from: W28, D53, E115, A106, K43, R44, R36, K123, C35, C104, C228, C230, E95, D129, D162, E205, D239, D271, E255, E315, E356, N87 and N157 within SEQ ID NO: 3. In some embodiments, the mutation is selected from mutation of at least one amino acid selected from: W2, D27, E89, A80, K17, R18, R10, K97, C9, C78, C202, C204, E69, D103, D136, E179, D213, D245, E229, E289, E330, N61 and N131 within SEQ ID NO: 4. In some embodiments, the mutation is W28A or W2A. In some embodiments, the mutation is D53T or D27T. In some embodiments, the mutation is E115S or E89S. In some embodiments, the mutation is A106I or A80I. In some embodiments, the mutation is K43A or K17A. In some embodiments, the mutation is R44A or R18A. In some embodiments, the mutation is K123A or K97A. In some embodiments, the mutation is R123A or R97A. In some embodiments, the mutation is C35V or C9V. In some embodiments, the mutation is C54A or C28A. In some embodiments, the mutation is C104V or C78V. In some embodiments, the mutation is C228A, C230A, C202A or C204A. In some embodiments, the mutation is C230V, C232V, C204V or C206V. In some embodiments, the mutation is E95G or E69G. In some embodiments, the mutation is D129G or D103G. In some embodiments, the mutation is D162G or D136G. In some embodiments, the mutation is E205G or E179G. In some embodiments, the mutation is D239G, D241G, D213G or D215G. In some embodiments, the mutation is D271G, D273G, D245G or D247G. In some embodiments, the mutation is E255G, E257G, E229G or E231G. In some embodiments, the mutation is E315G, E317G, E289G, or E291G. In some embodiments, the mutation is E356G, E358G, E330G or E332G. In some embodiments, the mutation is to alanine. In some embodiments, the mutation is mutation to glycine.

In some embodiments, the DSG1 extracellular domain comprises SEQ ID NO: 2 and the mutation is selected from: W2A, D27T, E89S, A80I, K17A, R18A, R10A, R97A, C9V, C9S, C28A, C28S, C78V, C78S, C204A, C204S, C206V, C206S, C426S, E69G, D103G, D136G, E179G, D215G, D247G, E231G, E291G, E332G, N61Q, and N131Q. In some embodiments, the at least one mutation is W2A. In some embodiments, the at least one mutation is D27T. In some embodiments, the at least one mutation is E89S. In some embodiments, the at least one mutation is W2A, D27T and E89S. In some embodiments, the at least one mutation is W2A and A80I. In some embodiments, the at least one mutation is K17A and R18A. In some embodiments, the at least one mutation is R10A, K17A, R18A and R97A. In some embodiments, the at least one mutation is C9V, C28A, C78V, C204A, C206V and C426S. In some embodiments, the at least one mutation is C9V, C28A, C78V, C204A, and C206V. In some embodiments, the at least one mutation is E69G, D103G and D136G. In some embodiments, the at least one mutation is E179G, D215G and D247G. In some embodiments, the at least one mutation is E231G, E291G and E332G. In some embodiments, the at least one mutation is N61Q and N131Q. In some embodiments, the at least one mutation is C9S, C28S, C78S, C204S and C206S. It will be understood that if SEQ ID NO: 1 is used, comprising the prodomain, then each of these amino acid positions will be increased by 26.

In some embodiments, the DSG3 extracellular domain comprises SEQ ID NO: 4 and the mutation is selected from: W2A, D27T, E89S, A80I, K17A, R18A, R10A, K97A, C9V, C9S, C78V, C78S, C202A, C202S, C204V, C204S, E69G, D103G, D136G, E179G, D213G, D245G, E229G, E289G, E330G, N61Q, and N131Q. In some embodiments, the at least one mutation is W2A. In some embodiments, the at least one mutation is D27T. In some embodiments, the at least one mutation is E89S. In some embodiments, the at least one mutation is W2A, D27T and E89S. In some embodiments, the at least one mutation is W2A and A80I. In some embodiments, the at least one mutation is K17A and R18A. In some embodiments, the at least one mutation is R10A, K17A, R18A and K97A. In some embodiments, the at least one mutation is C9V, C78V, C202A, and C204V. In some embodiments, the at least one mutation is E69G, D103G and D136G. In some embodiments, the at least one mutation is E179G, D213G and D245G. In some embodiments, the at least one mutation is E229G, E289G and E330G. In some embodiments, the at least one mutation is N61Q and N131Q. In some embodiments, the at least one mutation is C9S, C78S, C202S and C204S. It will be understood that if SEQ ID NO: 3 is used, comprising the prodomain, then each of these amino acid positions will be increased by 26.

In some embodiments, the fragment comprises an amino acid sequence selected from SEQ ID NO: 74-85, 90-103 and 108-113. In some embodiments, the fragment consists of an amino acid sequence selected from SEQ ID NO: 74-85, 90-103 and 108-113. In some embodiments, the fragment comprises SEQ ID NO: 74, 75, 76, 77, 78, 79, 80, 81, 82, 83, 84, 85, 90, 91, 92, 93, 94, 95, 96, 97, 98, 99, 100, 102, 103, 108, 109, 110, 111, 112 or 113. Each possibility represents a separate embodiment of the invention. In some embodiments, the fragment consists of SEQ ID NO: 74, 75, 76, 77, 78, 79, 80, 81, 82, 83, 84, 85, 90, 91, 92, 93, 94, 95, 96, 97, 98, 99, 100, 102, 103, 108, 109, 110, 111, 112 or 113. Each possibility represents a separate embodiment of the invention. In some embodiments, the fragment comprises SEQ ID NO: 81. In some embodiments, the fragment consists of SEQ ID NO: 81. In some embodiments, the fragment comprises SEQ ID NO: 82. In some embodiments, the fragment consists of SEQ ID NO: 82. In some embodiments, the fragment comprises SEQ ID NO: 90. In some embodiments, the fragment consists of SEQ ID NO: 90. In some embodiments, the fragment comprises SEQ ID NO: 100. In some embodiments, the fragment consists of SEQ ID NO: 100. In some embodiments, the fragment comprises SEQ ID NO: 108. In some embodiments, the fragment consists of SEQ ID NO: 108. In some embodiments, the extra cellular domain is from DSG1 and the fragment comprises or consists of any one of: SEQ ID NO: 74-85 and 90-93. In some embodiments, the extra cellular domain is from DSG3 and the fragment comprises or consists of any one of SEQ ID NO: 94-103 and 108-113. In some embodiments, the extra cellular domain is from DSG1 and the fragment comprises or consists of SEQ ID NO: 74, 75, 76, 77, 78, 79, 80, 81, 82, 83, 84, 85, 90, 91, 92 or 93. Each possibility represents a separate embodiment of the invention. In some embodiments, the extra cellular domain is from DSG3 and the fragment comprises or consists of SEQ ID NO: 94, 95, 96, 97, 98, 99, 100, 101, 102, 103, 108, 109, 110, 111, 112 or 113. Each possibility represents a separate embodiment of the invention. In some embodiments, the extra cellular domain is from DSG1 and the fragment comprises or consists of SEQ ID NO: 81. In some embodiments, the extra cellular domain is from DSG1 and the fragment comprises or consists of SEQ ID NO: 82. In some embodiments, the extra cellular domain is from DSG1 and the fragment comprises or consists of SEQ ID NO: 90. In some embodiments, the extra cellular domain is from DSG3 and the fragment comprises or consists of SEQ ID NO: 100. In some embodiments, the extra cellular domain is from DSG3 and the fragment comprises or consists of SEQ ID NO: 108.

In some embodiments, the mutation is a plurality of mutations. In some embodiments, a plurality is at least 2, 3, 4, 5, 6, 7, 8, 9 or 10 mutations. Each possibility represents a separate embodiment of the invention. In some embodiments, a plurality is 2. In some embodiments, a plurality is 4. In some embodiments, a plurality is 5. In some embodiments, the plurality of mutations is selected from the sets of mutation provided in Table 3. In some embodiments, the plurality of mutations is selected from the sets of mutation provided in Table 4. In some embodiments, the plurality of mutations is selected from the sets of mutation provided in Table 5. In some embodiments, the plurality of mutations comprises mutation of C35, C104, C228, and C230. In some embodiments, the mutations are in SEQ ID NO: 3. In some embodiments, the mutation are in SEQ ID NO: 1. In some embodiments, the plurality of mutations comprises mutation of C9, C78, C202, and C204. In some embodiments, the mutations are in SEQ ID NO: 4. In some embodiments, the mutations are in SEQ ID NO: 2. In some embodiments, the plurality of mutations comprises mutation of C35, C54, C104, C230, and C232. In some embodiments, the mutations are in SEQ ID NO: 1. In some embodiments, the mutations are in SEQ ID NO: 3. In some embodiments, the plurality of mutations comprises mutation of C9, C28, C78, C204, and C206 in SEQ ID NO: 2. In some embodiments, the plurality of mutations comprises mutation of C9, C28, C78, C204, and C206 in SEQ ID NO: 4. In some embodiments, the C is mutated to V. In some embodiments, the C is mutated to A. In some embodiments, a derivative is a derivative of a mutated fragment. In some embodiments, a derivative comprises at least 85% sequence identity to the mutated fragment and comprises the mutation.

In some embodiments, the fragment comprises an extracellular functional domain. In some embodiments, the functional domain is a cadherin domain. In some embodiments, the functional domain is a calcium binding domain. In some embodiments, the mutation is in an extracellular functional domain. In some embodiments, the mutation is in a cadherin domain. In some embodiments, the mutation alters binding to a cadherin. In some embodiments, the mutation decreases binding to a cadherin. In some embodiments, alters is decreases. In some embodiments, the mutation is in a calcium binding domain. In some embodiments, the mutation decreases binding to calcium. In some embodiments, decreases is abolishes. In some embodiments, decreases is by at least 5, 10, 15, 20, 25, 30, 40, 50, 60, 70, 75, 80, 90, 92, 95, 97, 99 or 100%. Each possibility represents a separate embodiment of the invention. In some embodiments, the extracellular functional domain comprises 4 cadherin domains. In some embodiments, the cadherin domains are EC1-EC4.

It will be understood by a skilled artisan that as the protein complex of the invention is meant to bind antibodies and B cells it would be advantageous not to bind to itself or to other copies of the therapeutic molecule. As such, mutations and truncations that decrease aggregation but do not interfere with autoantibody binding are advantageous. In some embodiments, the fragment comprises a truncation of the extracellular domain. In some embodiments, the fragment consists of a truncation of the extracellular domain. In some embodiments, the truncation lacks at least one extracellular functional domain. In some embodiments, the truncation lacks at least two extracellular functional domains. In some embodiments, the truncation lacks EC1. In some embodiments, the truncation lacks EC2. In some embodiments, the truncation lacks EC3. In some embodiments, the truncation lacks EC4. In some embodiments, the truncation lacks EC1 and EC2. In some embodiments, the truncation lacks EC2 and EC3. In some embodiments, the truncation lacks EC3 and EC4. In some embodiments, a functional domain is selected from SEQ ID NO: 114-117. In some embodiments, a functional domain is selected from SEQ ID NO: 118-121.

In some embodiments, a derivative is a derivative of the truncation. In some embodiments, the derivative comprises at least 85% identity to the truncation and does not further comprise a stretch of amino acids homologous/identical to a sequence from DSG1 or DSG3. Thus, it will be understood that a sequence with sequence identity to a truncation is not a sequence which is not truncated. In some embodiments, the truncation comprises at least one mutation. In some embodiments, the derivative comprises at least 85% identity to any one of SEQ ID NO: 74-85, 90-103 and 108-113 and comprises the mutation/s. In some embodiments, the derivative comprises at least 85% identity to any one of SEQ ID NO: 87, 90-91, 105, and 108-109 and are devoid of EC3 and EC4.

### Dimerization Domains

In some embodiments, dimerization domains are capable of dimerizing with each other. In some embodiments, the first dimerization domain is capable of dimerization with the second dimerization domain. In some embodiments, the first and second dimerization domains are capable of dimerizing with each other. In some embodiments, capable of dimerizing is configured to dimerize. In some embodiments, dimerization is under physiological conditions. In some embodiments, dimerization is within a bodily fluid. In some embodiments, the bodily fluid is blood. In some embodiments, the bodily fluid is plasma. In some embodiments, the bodily fluid is serum. In some embodiments, dimerization is within a subject. In some embodiments, dimerization is in vivo. In some embodiments, dimerization is in vitro.

As used herein, the term “dimerization domain” refers to an amino acid sequence that upon contacting another amino acid sequence (the other dimerization domain) binds to it to form a dimer. Dimerization domains are well known in the art, as many protein sequences are known to bind to each other. In some embodiments, dimerization comprises formation of a covalent bond between the dimerization domains. In some embodiments, dimerization comprises electrostatic binding. In some embodiments, dimerization does not comprise electrostatic binding. In some embodiments, dimerization is reversible. In some embodiments, dimerization is irreversible. In some embodiments, dimerization comprises a bond forming between the dimerization domains. In some embodiments, the bond is a chemical bond. In some embodiments, the bond is a disulfide bond. In some embodiments, the bond is a peptide bond. Examples of dimerization domain include the hinge domain of antibody heavy chains, the CH1/CL domains of antibody heavy/light chains, and the ECD domains of TCR alpha/beta to name but a few. Additionally, the upper hinge domain can be engineered with cysteine substitutions/mutations to serine in order to prevent dimerization. In some embodiments, the dimerization domain comprises or consists of the sequence EPKSSDKTHTCPPCP (SEQ ID NO: 124).

In some embodiments, the dimerization domain comprises or consists of an immunoglobulin (Ig) hinge domain. In some embodiments, an Ig hinge domain is a heavy chain hinge domain. In some embodiments, the Ig is a human Ig. In some embodiments, the immunoglobulin is elected from IgA, IgD, IgE, IgG and IgM. In some embodiments, the immunoglobulin is IgG. In some embodiments, the IgG is IgG1. In some embodiments, the IgG is IgG2. In some embodiments, the IgG is IgG3. In some embodiments, the IgG is selected from IgG1 and IgG3. In some embodiments, the IgG is IgG4. In some embodiments, the first and second dimerization domains are both Ig hinge domains. In some embodiments, the first and second dimerization domains are identical. In some embodiments, the first and second dimerization domains are at least 95% identical. In some embodiments, the first and second dimerization domains are at least 50, 55, 60, 65, 70, 75, 80, 85, 90, 95, 97, 99 or 100% identical. Each possibility represents a separate embodiment of the invention.

In some embodiments, the hinge domain comprises the amino acid sequence EPKSCDKTHTCPPCPAPELLGGP (SEQ ID NO: 125). In some embodiments, the hinge domain consists of the amino acid sequence of SEQ ID NO: 125. In some embodiments, the IgG1 hinge comprises or consists of SEQ ID NO: 125. In some embodiments, the hinge domain comprises the amino acid sequence EPKCCVECPPCPAPPAAAP (SEQ ID NO: 126). In some embodiments, the hinge domain consists of the amino acid sequence of SEQ ID NO: 126. In some embodiments, the IgG2 hinge comprises or consists of SEQ ID NO: 126. In some embodiments, the hinge domain comprises the amino acid sequence ESKYGPPCPPCPAPEFLGGP (SEQ ID NO: 127). In some embodiments, the hinge domain consists of the amino acid sequence of SEQ ID NO: 127. In some embodiments, the IgG4 hinge comprises or consists of SEQ ID NO: 127. In some embodiments, the hinge domain comprises the amino acid sequence ELKTPLGDTTHTCPRCPEPKSCDTPPPCPRCPEPKSCDTPPPCPRCPEPKSCDTPPPC PRCPAPELLGGP (SEQ ID NO: 128). In some embodiments, the hinge domain consists of the amino acid sequence of SEQ ID NO: 128. In some embodiments, the IgG3 hinge comprises or consists of SEQ ID NO: 128. In some embodiments, the hinge domain comprises a CPXCP (SEQ ID NO: 129) motif. In some embodiments, the X in SEQ ID NO: 129 is selected from P and R. In some embodiments, SEQ ID NO: 129 is CPPCP (SEQ ID NO: 130). In some embodiments, SEQ ID NO: 129 is CPRCP (SEQ ID NO: 131). In some embodiments, the hinge domain comprises EPKSCDKTHTCPPCP (SEQ ID NO: 132). It will thus be understood that the hinge region can be considered to end after the CPXCP motif.

In some embodiments, the dimerization domain comprises or consists of an Ig CH1 domain. In some embodiments, the dimerization domain comprises or consists of an Ig heavy chain CH1 domain. In some embodiments, the dimerization domain comprises or consists of an Ig light chain. In some embodiments, the dimerization domain comprises or consists of a light chain CL domain. In some embodiments, the CL domain is a CL kappa domain. In some embodiments, the CL domain is a CL lambda domain. It is well known in the art that the CH1 domain of the Ig heavy chain dimerizes with the light chain CL domain. In some embodiments, the first dimerization domain comprises or consists of a CH1 domain, and the second dimerization domain comprises or consists of a CL domain. In some embodiments, the first and second dimerization domains both comprise a hinge domain. In some embodiments, the first and second dimerization domains do not both comprise a CH1 domain. In some embodiments, the first and second dimerization domains do not both comprise a CL domain. In some the first and second polypeptide chains do not both comprise a CH1 domain. In some the first and second polypeptide chains do not both comprise a CL domain.

In some embodiments, an Ig CH1 domain comprises of the amino acid sequence ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKV (SEQ ID NO: 133). In some embodiments, an Ig CH1 domain consists of SEQ ID NO: 133. In some embodiments, SEQ ID NO: 133 is the IgG1 CH1 domain. In some embodiments, an Ig CH1 domain comprises of the amino acid sequence ASTKGPSVFPLAPCSRSTSESTAALGCLVKDYFPEPVTVSWNSGALTSGVHTFPAV LQSSGLYSLSSVVTVPSSNFGTQTYTCNVDHKPSNTKVDKTV (SEQ ID NO: 134). In some embodiments, an Ig CH1 domain consists of SEQ ID NO: 134. In some embodiments, SEQ ID NO: 134 is the IgG2 CH1 domain. In some embodiments, an Ig CH1 domain comprises of the amino acid sequence ASTKGPSVFPLAPCSRSTSGGTAALGCLVKDYFPEPVTVSWNSGALTSGVHTFPAV LQSSGLYSLSSVVTVPSSSLGTQTYTCNVNHKPSNTKVDKRV (SEQ ID NO: 135). In some embodiments, an Ig CH1 domain consists of SEQ ID NO: 135. In some embodiments, SEQ ID NO: 135 is the IgG3 CH1 domain. In some embodiments, an Ig CH1 domain comprises of the amino acid sequence ASTKGPSVFPLAPCSRSTSESTAALGCLVKDYFPEPVTVSWNSGALTSGVHTFPAV LQSSGLYSLSSVVTVPSSSLGTKTYTCNVDHKPSNTKVDKRV (SEQ ID NO: 136). In some embodiments, an Ig CH1 domain consists of SEQ ID NO: 136. In some embodiments, SEQ ID NO: 136 is the IgG4 CH1 domain.

In some embodiments, an Ig CL Kappa domain comprises of the amino acid sequence AAPSVFIFPPSDEQLKSGTASVVCLLNNFYPREAKVQWKVDNALQSGNSQESVTE QDSKDSTYSLSSTLTLSKADYEKHKVYACEVTHQGLSSPVTKSFNRGEC (SEQ ID NO: 137). In some embodiments, an Ig CL Kappa domain consists of SEQ ID NO: 137. In some embodiments, an Ig CL Lambda domain comprises of the amino acid sequence GQPKANPTVTLFPPSSEELQANKATLVCLISDFYPGAVTVAWKADGSPVKAGVET TKPSKQSNNKYAASSYLSLTPEQWKSHRSYSCQVTHEGSTVEKTVAPTECS (SEQ ID NO: 138). In some embodiments, an Ig CL Lambda domain consists of SEQ ID NO: 138.

### Effector Moiety

In some embodiments, the composition comprises an effector moiety. In some embodiments, the first polypeptide chain comprises an effector moiety. In some embodiments, the second polypeptide chain comprises an effector moiety. In some embodiments, both the first and second polypeptide chains comprise an effector moiety. The term “moiety”, as used herein, relates to a part of a molecule that may include either whole functional groups or parts of functional groups as substructures. The term “moiety” may also refer to part of a molecule that exhibits a particular set of chemical and/or pharmacologic characteristics which are similar to the corresponding molecule. As used herein, the term “effector moiety” refers to a molecule or fragment of a molecule that carriers out a cytotoxic effect. In some embodiments, an effector moiety is an effector molecule.

In some embodiments, the effector moiety is capable of inducing a cytotoxic effect. In some embodiments, the effector moiety is configured to induce a cytotoxic effect. In some embodiments, the effector moiety is capable of inducing death. In some embodiments, the effector moiety is configured to induce death. In some embodiments, death is cell death. In some embodiments, death is apoptosis. In some embodiments, death is necrosis. In some embodiments, death is cell mediated death. In some embodiments, death is phagocytosis. In some embodiments, the cytotoxic effect is against a target cell. In some embodiments, death is in a target cell. In some embodiments, the cytotoxic effect is upon binding. In some embodiments, death is upon binding. In some embodiments, the cytotoxic effect is against a target cell binding the composition. In some embodiments, the death is death of a target cell binding the composition. In some embodiments, the cytotoxic effect is against a cell bound by the protein complex. In some embodiments, the cytotoxic effect is against a cell binding the protein complex. In some embodiments, the death is death of a cell bound by the protein complex. In some embodiments, the death is death of a cell binding the protein complex. In some embodiments, the cytotoxic effect is a direct effect. In some embodiments, the cytotoxic effect is an indirect effect. In some embodiments, binding the composition is binding the fragments. In some embodiments, binding the protein complex is binding the fragments. In some embodiments, the fragments are at least one of the fragments. In some embodiments, the fragments are one of the fragments. In some embodiments, the fragments are both of the fragments.

In some embodiments, the effector moiety is a cytotoxic moiety. In some embodiments, the effector moiety is a toxin. In some embodiments, the effector moiety is a poison. In some embodiments, the effector moiety is chemotherapeutic. In some embodiments, the effector moiety is an anticancer agent. In some embodiments, the effector moiety is an engager. In some embodiments, an engager binds a cytotoxic cell. In some embodiments, binding a cytotoxic cell is recruiting a cytotoxic cell. In some embodiments, binds is bound by.

In some embodiments, the effector moiety recruits a cytotoxic agent. In some embodiments, the cytotoxic agent is a cytotoxic cell. In some embodiments, the cytotoxic cell is an immune cell. In some embodiments, the immune cell is a T cell. In some embodiments, the immune cell is a natural killer (NK) cell. In some embodiments, the immune cell is a macrophage. In some embodiments, the T cell is a cytotoxic T cell. In some embodiments, the T cell is a CD8 positive T cell. In some embodiments, the effector moiety induces antibody-dependent cell cytotoxicity (ADCC). In some embodiments, the effector moiety induces complement-dependent cytotoxicity (CDC).

In some embodiments, the effector moiety binds a receptor on a cell surface of the cytotoxic cell. Examples of receptors include, but are not limited to CD3, CD8, CD56, CD14 and CD16. In some embodiments, the receptor is a marker of the cytotoxic cell. In some embodiments, the receptor is unique to the cytotoxic cell. In some embodiments, the receptor is CD3. In some embodiments, the effector moiety is an agent that binds CD3. In some embodiments, the engager is an agent that binds CD3. In some embodiments, CD3 is human CD3. In some embodiments, the agent that binds CD3 is an anti-CD3 antibody or antigen binding fragment thereof. In some embodiments, the receptor is CD16. In some embodiments, the effector moiety is an agent that binds CD16. In some embodiments, the engager is an agent that binds CD16. In some embodiments, CD16 is human CD16. In some embodiments, the agent that binds CD16 is an anti-CD16 antibody or antigen binding fragment thereof. In some embodiments, the antibody of antigen binding fragment thereof is a single chain antibody. In some embodiments, the antibody of antigen binding fragment thereof is a single domain antibody. In some embodiments, the antibody of antigen binding fragment thereof is a single chain variable fragment (scFv). Anti-CD3 agents are well known in the art and any such binding agent may be used. For example, the anti-human CD3 scFv known as OKT3 may be used as the agent. In some embodiments, the cytotoxic moiety is selected from alpha-amanitin, a radioactive moiety and an anti-CD3 binding agent. Other example of human anti-CD3 antibodies include: Muromonab (trade name Orthoclone OKT3), a murine monoclonal anti-human CD3 antibody (DrugBank Accession Number DB00075); Teplizumab, a humanized version of the murine OKT3 anti-CD3 monoclonal antibody (DrugBank Accession Number DB06606); UCHT1, a murine monoclonal anti-human CD3 antibody; UCHT1 variant-9, a humanized version of the UCHT1 clone and the bi-specific CD19-CD3 Blinatumomab (DrugBank Accession Number DB09052). Examples of human anti-CD16 include: AFM13, a bispecific tetravalent Innate Cell Engager (ICE®) targeting CD30 on tumor cells and CD16A on NK cells and macrophages and GTB-3550 (CD16/IL-15/CD33) a tri-specific killer cell engager.

In some embodiments, the composition comprises an Fc region. In some embodiments, the effector moiety is not an Fc region. In some embodiments, not an Fc region is not an unmodified Fc region. In some embodiments, the composition comprises an effector moiety that is not an Fc region. In some embodiments, the composition comprises an effector moiety other than an Fc region. In some embodiments, the composition is devoid of an Fc region. In some embodiments, the protein comprises an effector moiety that is not an Fc region. In some embodiments, the protein comprises an effector moiety other than an Fc region. In some embodiments, the protein is devoid of an Fc region. In some embodiments, the engager is an Fc region. In some embodiments, the engager is not an Fc region. In some embodiments, the composition comprises an effector moiety that is superior at killing as compared to an Fc. In some embodiments, superior at killing is superior at killing B cells. In some embodiments, an Fc is an unmodified Fc. In some embodiments, an Fc is an unmutated Fc. In some embodiments, an Fc is a naturally occurring Fc. In some embodiments, an Fc is a human Fc. In some embodiments, a superior Fc is an Fc comprising at least one mutation that increases ADCC. In some embodiments, an Fc region is an Fc domain. In some embodiments, an Fc region is an Fc fragment. In some embodiments, the first polypeptide chain comprises an Fc region. In some embodiments, the second polypeptide chain comprises an Fc region. In some embodiments, both the first and second polypeptide chains comprise an Fc region. In some embodiments, the Fc region is an Fc region of an antibody heavy chain. In some embodiments, the antibody heavy chain is a human antibody heavy chain. In some embodiments, the heavy chain is an IgG heavy chain. In some embodiments, the IgG is selected from IgG1, IgG2, IgG3 and IgG4. In some embodiments, the IgG is selected from IgG1 and IgG3. In some embodiments, the IgG is IgG1. In some embodiments, the IgG is IgG2. In some embodiments, the IgG is IgG3. In some embodiments, the IgG is IgG4.

In some embodiments, the Fc region is capable of inducing a cytotoxic effect. In some embodiments, the Fc domain comprises DKTHTCPPCPAPELLGGPSVFLFPPKPKDTLMISRTPEVTCVVVDVSHEDPEVKEN WYVDGVEVHNAKTKPREEQYNSTYRVVSVLTVLHQDWLNGKEYKCKVSNKALP APIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSL SLSPGK (SEQ ID NO: 164). In some embodiments, the Fc domain comprises EPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSN KALPAPIEKTISKAKGQPREPQVYTLPPSRDELTKNQVSLTCLVKGFYPSDIA VEWE SNGQPENNYKTTPPVLDSDGSFFLYSKLTVDKSRWQQGNVFSCSVMHEALHNHY TQKSLSLSPGK (SEQ ID NO: 171). It will be understood that SEQ ID NO: 171 contains 5 additional N-terminal amino acids as compared to SEQ ID NO: 164. As such, while number herein is given with respect to SEQ ID NO: 164 the numbering for SEQ ID NO: 171 can be found by adding 5. In some embodiments, the Fc region is capable of inducing a cytotoxic effect. In some embodiments, the Fc domain comprises DKTHTCPPCPAPELLGGPSVFLFPPKPKDTLMISRTPEVTCVVVDVSHEDPEVKEN WYVDGVEVHNAKTKPREEQYNSTYRVVSVLTVLHQDWLNGKEYKCKVSNKALP APIEKTISKAKGQPREPQVYTLPPSREEMTKNQVSLTCLVKGFYPSDIAVEWESNG QPENNYKTTPPVLDSDGSFFLYSKLTVDKSRWQQGNVFSCSVMHEALHNHYTQK SLSLSPGK (SEQ ID NO: 165). In some embodiments, the Fc domain comprises EPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSN KALPAPIEKTISKAKGQPREPQVYTLPPSREEMTKNQVSLTCLVKGFYPSDIAVEW ESNGQPENNYKTTPPVLDSDGSFFLYSKLTVDKSRWQQGNVFSCSVMHEALHNH YTQKSLSLSPGK (SEQ ID NO: 155). It will be understood that SEQ ID NO: 155 contains 5 additional N-terminal amino acids as compared to SEQ ID NO: 165. As such, while number herein is given with respect to SEQ ID NO: 165 (or SEQ ID NO: 164 which is equivalent) the numbering for SEQ ID NO: 155 can be found by adding 5. SEQ ID NO: 164 and SEQ ID NO: 165 differ by two amino acids. The two sequences can be interchanged and when mutations are given with respect to SEQ ID NO: 164 it will be understood that they apply also to SEQ ID NO: 165 and vice-versa. So too SEQ ID NO: 171 and SEQ ID NO: 155 also differ by only two amino acids and these two sequences can be interchanged.

In some embodiments, the Fc domain consists of SEQ ID NO: 164. In some embodiments, the Fc domain of IgG1 comprises or consists of SEQ ID NO: 164. In some embodiments, the Fc domain comprises or consists of a sequence with at least 70, 75, 80, 85, 90, 93, 95, 97, or 99% homology to SEQ ID NO: 164. Each possibility represents a separate embodiment of the invention. In some embodiments, the Fc domain consists of SEQ ID NO: 171. In some embodiments, the Fc domain of IgG1 comprises or consists of SEQ ID NO: 171. In some embodiments, the Fc domain comprises or consists of a sequence with at least 70, 75, 80, 85, 90, 93, 95, 97, or 99% homology to SEQ ID NO: 171. Each possibility represents a separate embodiment of the invention. In some embodiments, the Fc domain consists of SEQ ID NO: 165. In some embodiments, the Fc domain of IgG1 comprises or consists of SEQ ID NO: 165. In some embodiments, the Fc domain comprises or consists of a sequence with at least 70, 75, 80, 85, 90, 93, 95, 97, or 99% homology to SEQ ID NO: 165. Each possibility represents a separate embodiment of the invention. In some embodiments, the Fc domain consists of SEQ ID NO: 155. In some embodiments, the Fc domain of IgG1 comprises or consists of SEQ ID NO: 155. In some embodiments, the Fc domain comprises or consists of a sequence with at least 70, 75, 80, 85, 90, 93, 95, 97, or 99% homology to SEQ ID NO: 155. Each possibility represents a separate embodiment of the invention.

In some embodiments, the Fc region is capable of inducing a cytotoxic effect. In some embodiments, the Fc region is configured to induce a cytotoxic effect. In some embodiments, the cytotoxic effect is against a target cell. In some embodiments, the cytotoxic effect is upon binding. In some embodiments, the cytotoxic effect is against a cell bound by the protein complex. In some embodiments, the cytotoxic effect is against a cell binding the protein complex. In some embodiments, the cytotoxic effect is mediated by immune cell binding to the Fc region. In some embodiments, the cytotoxic effect is mediated by immune cell activation by the Fc region. In some embodiments, the cytotoxic effect is mediated by immune cell recruitment by the Fc region. In some embodiments, the immune cell is a T cell. In some embodiments, the immune cell is a natural killer (NK) cell. In some embodiments, the immune cell is a macrophage. In some embodiments, the T cell is a cytotoxic T cell. In some embodiments, the T cell is a CD8 positive T cell. In some embodiments, the Fc region induces antibody-dependent cell cytotoxicity (ADCC). In some embodiments, the Fc region induces complement-dependent cytotoxicity (CDC).

In some embodiments, the Fc region comprises an Ig CH2 domain. In some embodiments, the Fc region comprises an Ig heavy chain CH2 domain. In some embodiments, the Fc region comprises an Ig CH3 domain. In some embodiments, the Fc region comprises an Ig heavy chain CH3 domain. In some embodiments, the Fc region comprises or consists of both an Ig CH2 domain and Ig CH3 domain. In some embodiments, the Fc region comprises or consists of both an Ig heavy chain CH2 and an Ig heavy chain CH3 domain. In some embodiments, the first chain comprises a first portion of an Fc region and the second chain comprises a second portion of the Fc region. In some embodiments, the first portion comprises a CH2 domain, a CH3 domain or both. In some embodiments, the second portion comprises a CH2 domain, a CH3 domain or both. In some embodiments, interface of the first portion of an Fc region and the second portion of an Fc region produces a functional Fc region. In some embodiments, interface comprises contact. In some embodiments, interface comprises adjacent positioning. In some embodiments, interface comprises formation of the protein complex of the invention. In some embodiments, interface comprises dimerization of the first and second dimerization domains. In some embodiments, the CH2 domain is an Ig CH2 domain. In some embodiments the CH2 domain is a heavy chain CH2 domain. In some embodiments, the CH3 domain is an Ig CH3 domain. In some embodiments, the CH3 domain is a heavy chain CH3 domain.

In some embodiments, a CH2 domain comprises the amino acid sequence SVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAK (SEQ ID NO: 139). In some embodiments, the CH2 domain consists of SEQ ID NO: 139. In some embodiments, SEQ ID NO: 139 is the IgG1 CH2 domain. In some embodiments, a CH2 domain comprises the amino acid sequence SVFLFPPKPKDTLMISRTPEVTCVVVDVSHEDPEVQFNWYVDGVEVHNAKTKPRE EQFNSTFRVVSVLTVVHQDWLNGKEYKCKVSNKGLPAPIEKTISKTK (SEQ ID NO: 140). In some embodiments, the CH2 domain consists of SEQ ID NO: 140. In some embodiments, SEQ ID NO: 40 is the IgG2 CH2 domain. In some embodiments, a CH2 domain comprises the amino acid sequence SVFLFPPKPKDTLMISRTPEVTCVVVDVSQEDPEVQFNWYVDGVEVHNAKTKPRE EQFNSTYRVVSVLTVLHQDWLNGKEYKCKVSNKGLPSSIEKTISKAK (SEQ ID NO: 141). In some embodiments, the CH2 domain consists of SEQ ID NO: 141. In some embodiments, SEQ ID NO: 141 is the IgG4 CH2 domain. In some embodiments, a CH2 domain comprises the amino acid sequence SVFLFPPKPKDTLMISRTPEVTCVVVDVSHEDPEVQFKWYVDGVEVHNAKTKPRE EQYNSTFRVVSVLTVLHQDWLNGKEYKCKVSNKALPAPIEKTISKTK (SEQ ID NO: 142). In some embodiments, the CH2 domain consists of SEQ ID NO: 142. In some embodiments, SEQ ID NO: 142 is the IgG3 CH2 domain.

In some embodiments, a CH3 domain comprises the amino acid sequence GQPREPQVYTLPPSREEMTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO: 143). In some embodiments, a CH3 domain comprises the amino acid sequence GQPREPQVYTLPPSRDELTKNQVSLTCLVKGFYPSDIA VEWESNGQPENNYKTTPP VLDSDGSFFLYSKLTVDKSRWQQGNVFSCSVMHEALHNHYTQKSLSLSPGK (SEQ ID NO: 144). In some embodiments, the CH3 domain consists of SEQ ID NO: 143. In some embodiments, the CH3 domain consists of SEQ ID NO: 144. In some embodiments, SEQ ID NO: 143 is the IgG1 CH3 domain. In some embodiments, SEQ ID NO: 144 is the IgG1 CH3 domain. In some embodiments, the SEQ ID NO: 143 sequence is the sequence found predominantly is humans of European and American descent. In some embodiments, SEQ ID NO: 144 is the sequence found predominantly in humans of Asian descent. In some embodiments, a CH3 domain the comprises amino acid sequence GQPREPQVYTLPPSREEMTKNQVSLTCLVKGFYPSDISVEWESNGQPENNYKTTPP MLDSDGSFFLYSKLTVDKSRWQQGNVFSCSVMHEALHNHYTQKSLSLSPGK (SEQ ID NO: 145). In some embodiments, the CH3 domain consists of SEQ ID NO: 145. In some embodiments, SEQ ID NO: 145 is the IgG2 CH3 domain. In some embodiments, a CH3 domain comprises the amino acid sequence GQPREPQVYTLPPSQEEMTKNQVSLTCLVKGFYPSDIAVEWESNGQPENNYKTTPP VLDSDGSFFLYSRLTVDKSRWQEGNVFSCSVMHEALHNHYTQKSLSLSLGK (SEQ ID NO: 146). In some embodiments, the CH3 domain consists of SEQ ID NO: 146. In some embodiments, SEQ ID NO: 146 is the IgG4 CH3 domain. In some embodiments, a CH3 domain comprises the amino acid sequence GQPREPQVYTLPPSREEMTKNQVSLTCLVKGFYPSDIAVEWESSGQPENNYNTTPP MLDSDGSFFLYSKLTVDKSRWQQGNIFSCSVMHEALHNRFTQKSLSLSPGK (SEQ ID NO: 147). In some embodiments, the CH3 domain consists of SEQ ID NO: 147. In some embodiments, SEQ ID NO: 147 is the IgG3 CH3 domain.

In some embodiments, the Fc comprises a mutation. In some embodiments, a CH3 domain comprises a mutation. In some embodiments, the first CH3 domain comprises a first mutation. In some embodiments, the second CH3 domain comprises a second mutation. In some embodiments, a CH2 domain comprises a mutation. In some embodiments, the first CH2 domain comprises a first mutation. In some embodiments, the second CH2 domain comprises a second mutation. In some embodiments, the CH2 and CH3 domains both comprise mutations. In some embodiments, the first CH2 domain and first CH3 domains each comprise a first mutation. In some embodiments, the second CH2 domain and the second CH3 domain each comprise a second mutation. In some embodiments, the mutations inhibit homodimerization of the first polypeptide chain. In some embodiments, the first mutation inhibits homodimerization of the first polypeptide chain. In some embodiments, the mutations inhibit homodimerization of the second polypeptide chain. In some embodiments, the second mutation inhibits homodimerization of the second polypeptide chain. In some, embodiments, the mutations permit heterodimerization. In some embodiments, the mutations permit heterodimerization of the first and second chains. In some embodiments, permitting is promoting. In some embodiments, permitting is enhancing.

Mutations that promote heavy chain heterodimerization and/or inhibit homodimerization are well known in the art. Any such mutations or alterations may be used for constructing the polypeptides of the invention. In some embodiments, a region from an IgG is replaced with a region from an IgA. In some embodiments, a region from a TCRa is inserted into the first CH3 domain and a region from TCRb is inserted in to the second CH3 domain. In some embodiments, the mutation is insertion of a region from a TCR. In some embodiments, the TCR is selected from TCRa and TCRb. In some embodiments, the mutation is insertion of a region from a different Ig. Examples of these mutations can be found in Table 1. In some embodiments, the mutation is selected from a mutation in Table 1. In some embodiments, the first mutation is selected from a group of mutation provided in a row and the second column of Table 1 and the second mutation is the group of mutations provided in that same row of Table 1 in the third column. The mutations in Table 1 are provided with the Kabat numbering for IgG1 unless otherwise stated; corresponding mutations can be made in other IGs and specifically in other IgGs. In some embodiments, the first mutation is T366Y, and the second mutation is Y407T. In some embodiments, the first mutation is S354C and T366W and the second mutation is Y349C, T366S, L368A, and Y407V. In some embodiments, the first mutation is S364H and F405A and the second mutation is Y349T and T392F. In some embodiments, the first mutation is T350V, L351Y, F405A, and Y407V and the second mutation is T350V, T366L, K392L, and T394W. In some embodiments, the first mutation is K392D, and K409D and the second mutation is E356K, and D399K. In some embodiments, the first mutation is D221E, P228E, and L368E and the second mutation is D221R, P228R, and K409R. In some embodiments, the first mutation is K360E, and K409W and the second mutation is Q347R, D399V, and F405T. In some embodiments, the first mutation is K360E, K409W, and Y349C and the second mutation is Q347R, D399V, F405T, and S354C. In some embodiments, the first mutation is F405L and the second mutation is K409R. In some embodiments, the first mutation is K360D, D399M, and Y407A and the second mutation is E345R, Q347R, T366V, and K409V. In some embodiments, the first mutation is Y349S, K370Y, T366M, and K409V and the second mutation is E356G, E357D, S364Q, and Y407A. In some embodiments, the first mutation is T366K, and the second mutation is selected from C351D, Y349E, Y349D, L368E, L368D, Y349E and R355E, Y349E and R355D, Y349D and R355E, and Y349D and R355D. In some embodiments, the first mutation is T366K and C351K and the second mutation is selected from C351D, Y349E, Y349D, L368E, L368D, Y349E and R355E, Y349E and R355D, Y349D and R355E, and Y349D and R355D. In some embodiments, the first mutation is L351D and L368E and the second mutation is L351K and T366K. In some embodiments, the first mutation is L368D and K370S and the second mutation is E357Q and S364K. In some embodiments, the first mutation is T366W, and the second mutation is T366S, L368A and Y407V. In some embodiments, the Ig is IgG2, and the first mutation is C223E, P228E, and L368E and the second mutation is C223R, E225R, P228R, and K409R. In some embodiments, the first mutation is S354C or T366W and the second mutation is Y349C, T366S, L368A, or Y407V. In some embodiments, the first mutation is S364H or F405A and the second mutation is Y349T or T392F. In some embodiments, the first mutation is T350V, L351Y, F405A, or Y407V and the second mutation is T350V, T366L, K392L, or T394W. In some embodiments, the first mutation is K392D, or K409D and the second mutation is E356K, or D399K. In some embodiments, the first mutation is D221E, P228E, or L368E and the second mutation is D221R, P228R, or K409R. In some embodiments, the first mutation is K360E, or K409W and the second mutation is Q347R, D399V, or F405T. In some embodiments, the first mutation is K360E, K409W, or Y349C and the second mutation is Q347R, D399V, F405T, or S354C. In some embodiments, the first mutation is K360D, D399M, or Y407A and the second mutation is E345R, Q347R, T366V, or K409V. In some embodiments, the first mutation is Y349S, K370Y, T366M, or K409V and the second mutation is E356G, E357D, S364Q, or Y407A. In some embodiments, the first mutation is L351D or L368E and the second mutation is L351K or T366K. In some embodiments, the first mutation is L368D or K370S and the second mutation is E357Q or S364K. In some embodiments, the first mutation is T366W, and the second mutation is T366S, L368A or Y407V. In some embodiments, the Ig is IgG2, and the first mutation is C223E, P228E, or L368E and the second mutation is C223R, E225R, P228R, or K409R. In some embodiments, the CH3 domain comprises or consists of GQPREPQVYTLPPSREEMTKNQVSLWCLVKGFYPSDIA VEWESNGQPENNYKTTP PVLDSDGSFFLYSKLTVDKSRWQQGNVFSCSVMHEALHNHYTQKSLSLSPGK (SEQ ID NO: 148). In some embodiments, the CH3 domain comprises or consists of GQPREPQVYTLPPSREEMTKNQVSLSCAVKGFYPSDIAVEWESNGQPENNYKTTPP VLDSDGSFFLVSKLTVDKSRWQQGNVFSCSVMHEALHNHYTQKSLSLSPGK (SEQ ID NO: 149). In some embodiments, the CH3 domain comprises or consists of GQPREPQVYTLPPSREEMTKNQVSLYCLVKGFYPSDIAVEWESNGQPENNYKTTP PVLDSDGSFFLYSKLTVDKSRWQQGNVFSCSVMHEALHNHYTQKSLSLSPGK (SEQ ID NO: 150). In some embodiments, the CH3 domain comprises or consists of GQPREPQVYTLPPSREEMTKNQVSLTCLVKGFYPSDIAVEWESNGQPENNYKTTPP VLDSDGSFFLTSKLTVDKSRWQQGNVFSCSVMHEALHNHYTQKSLSLSPGK (SEQ ID NO: 151).

TABLE 1
Mutations for enhancing heterodimerization and
inhibiting homodimerization of CH3 domains.

| | Strategy | CH3 domain Chain 1 | CH3 domain Chain 2 |
|---|---|---|---|
| 1 | Knobs-into-holes (Y-T) | T366Y | Y407T |
| 2 | Knobs-into-holes (CW- | S354C, T366W | Y349C, T366S, L368A, |
| | CSAV) | | Y407V |
| 3 | HA-TF | S364H, F405A | Y349T, T394F |
| 4 | ZW1 (VYAV-VLLW) | T350V, L351Y, F405A, | T350V, T366L, K392L, |
| | | Y407V | T394W |
| 5 | CH3 charge pairs (DD- | K392D, K409D | E356K, D399K |
| | KK) | | |
| 6 | Hinge/CH3 charge (EEE- | D221E, P228E, L368E | D221R, P228R, K409R |
| | RRR) | | |
| 7 | EW-RVT | K360E, K409W, | Q347R, D399V, F405T |
| 8 | EW-RVTS-S | K360E, K409W, Y349C | Q347R, D399V, F405T, |
| | | | S354C |
| 9 | (L-R) | F405L | K409R |
| 10 | 7.8.60 (DMA-RRVV) | K360D, D399M, Y407A | E345R, Q347R, T366V, |
| | | | K409V |
| 11 | 20.8.34 (SYMV-GDQA) | Y349S, K370Y, | E356G, E357D, S364Q, |
| | | T366M, K409V | Y407A |
| 12 | Electrostatic steering | 366K or 366K + C351K | C351D or E or D at 349, |
| | effects | | 368, 349, or 349 + 355 |
| 13 | “DEKK” | L351D and L368E | L351K and T366K |
| 14 | XmAb | L368D/K370S | E357Q/S364K |
| 15 | KiH | T366W | T366S/L368A/Y407V |
| 16 | IgG2 hinge/CH3 charge | IgG2: C223E, P228E, | IgG2: C223R, E225R, |
| | (EEE-RRRR) | L368E | P228R, K409R |
| 17 | SEEDbody | IgG/A chimera | IgG/A chimera |
| 18 | BEAT | residues from TCRa | residues from TCRb |
| | | interface | interface |

In some embodiments, the Fc domain comprises at least one mutation that increases effector function. In some embodiments, the Fc domain comprises at least one mutation that increases CDC, ADCC or both. In some embodiments, the Fc domain comprises at least one mutation that increases CDC. In some embodiments, the Fc domain comprises at least one mutation that increases ADCC. In some embodiments, the Fc domain comprises at least one mutation that increases antibody effector function. In some embodiments, the Fc domain comprises at least one mutation that increases antibody stability. In some embodiments, stability is half-life. In some embodiments, half-life is circulation half-life. In some embodiments, half-life is half-life in blood. In some embodiments, blood is serum.

In some embodiments, the mutation reduces effector function. In some embodiments, effector function comprises ADCC, CDC or both. In some embodiments, reduced effector function comprises reduced cytotoxicity. In some embodiments, reduces is abolishes. In some embodiments, the Fc is from IgG1 or IgG3 and the mutation reduces effector function. In some embodiments, the Fc is from IgG1 and comprises at least one mutation that reduces effector function. Mutations that reduce effector function are well known in the art and any such mutation can be used. Examples of such mutations can be found in Saunders, 2019, “Conceptual approaches to modulating antibody effector functions and circulation half-life” Front Immunol., June 7; 10:1296, herein incorporated by reference in its entirety.

It will be known by a skilled artisan that IgG2 and IgG4 possess greatly reduced effector function and are not generally cytotoxic in nature. Additionally, mutations such as S228P and L235E in IgG4 are known to reduce effector function even more. Further, mutations that reduce the cytotoxicity/effector function of IgG1 and IgG3 are well known in the art. In some embodiments, the IgG comprises at least one mutation. In some embodiments, the mutation is a plurality of mutations. In some embodiments, the mutation decreases cytotoxicity. In some embodiments, the mutation increases stability. In some embodiments, the mutation decreases aggregation. In some embodiments, the Fc domain comprises at least one mutation that decreases antibody effector function. In some embodiments, the Fc domain comprises at least one mutation that decreases ADCC. In some embodiments, the at least one mutation that decreases ADCC is a LALA mutation. As used herein, the LALA mutation refers to mutation of two successive leucine residues to alanine residues. In some embodiments, the LALA mutation is within the hinge domain. In some embodiments, the hinge domain is the hinge domain of IgG1. In some embodiments, the LALA mutation is mutation of L19 and L20 of SEQ ID NO: 125 to A19 and A20. In some embodiments, a LALA mutation hinge comprises an L19A and an L20A mutation of SEQ ID NO: 125. In some embodiments, the Fc domain comprises a hinge domain comprising SEQ ID NO: 162. In some embodiments, an Fc domain comprising a LALA mutation comprises SEQ ID NO: 162. In some embodiments, the Fc domain comprises a hinge domain consisting of SEQ ID NO: 162. In some embodiments, a LALA mutated hinge domain consists of SEQ ID NO: 162. In some embodiments, the LALA mutation is a L234A and L235A mutation of the Fc. In some embodiments, the at least one mutation that decreases ADCC is a N297A mutation. In some embodiments, the N297A mutation is within the CH2 domain. In some embodiments, the N297A mutation is mutation of asparagine 59 of SEQ ID NO: 139 to alanine. In some embodiments, an N297A mutated CH2 domain comprises an N59A mutation of SEQ ID NO: 139. In some embodiments, the Fc domain comprises a CH2 domain comprising SEQ ID NO: 163. In some embodiments, the Fc domain comprises a CH2 domain consisting of SEQ ID NO: 163. In some embodiments, the N297A mutated CH2 domain consists of SEQ ID NO: 163.

In some embodiments, the plurality of mutations that decreases cytotoxicity comprise the LALA mutations. In some embodiments, the plurality of mutations that decreases cytotoxicity comprise the PG-LALA mutations. In some embodiments, the mutation is mutation of proline 329 of the IgG1 human heavy chain to glycine (P329G). In some embodiments, the P to G mutation is mutation of P109 of SEQ ID NO: 164 to G. In some embodiments, the mutation is mutation of leucine 234 of the IgG1 human heavy chain to alanine (L234A). In some embodiments, the L to A mutation is mutation of L14 of SEQ ID NO: 164 to A. In some embodiments, the mutation is mutation of leucine 235 of the IgG1 human heavy chain to alanine (L235A). In some embodiments, the L to A mutation is mutation of L15 of SEQ ID NO: 164 to A. In some embodiments, the plurality of mutation comprises P109G, L14A and L15A of SEQ ID NO: 164. In some embodiments, the plurality of mutation comprises L14A and L15A of SEQ ID NO: 164. In some embodiments, the plurality of mutation comprises P329G, L234A and L235A of the IgG1 human heavy chain. In some embodiments, the plurality of mutation comprises L234A and L235A of the IgG1 human heavy chain. It will be understood by a skilled artisan that parallel mutation can also be performed in the IgG3 heavy chain or the heavy chains of non-human IgGls. In some embodiments, the plurality of mutations that decreases cytotoxicity comprise the YTE mutations. In some embodiments, the mutation is mutation of methionine 252 of the IgG1 human heavy chain to tyrosine (M252Y). In some embodiments, the M to Y mutation is mutation of M32 of SEQ ID NO: 164 to Y. In some embodiments, the mutation is mutation of serine 254 of the IgG1 human heavy chain to threonine (S254T). In some embodiments, the S to T mutation is mutation of S34 of SEQ ID NO: 164 to T. In some embodiments, the mutation is mutation of threonine 256 of the IgG1 human heavy chain to glutamic acid (T256E). In some embodiments, the T to E mutation is mutation of T36 of SEQ ID NO: 164 to E. In some embodiments, the plurality of mutation comprises M32Y, S34T and T36E of SEQ ID NO: 164. In some embodiments, the plurality of mutation comprises M252Y, S254T and T256E of the IgG1 human heavy chain. In some embodiments, the mutation is mutation of asparagine 297 of the IgG1 human heavy chain (N297). In some embodiments, the asparagine is mutated to alanine (N297A). In some embodiments, the asparagine is mutated to glutamine (N297Q). In some embodiments, the asparagine is N77 of SEQ ID NO: 164 (N77A or N77Q).

In some embodiments, the mutation increases the half-life of the molecule, peptide, polypeptide or protein complex. In some embodiments, a mutation that increases half-life is a mutation that increases binding to the neonatal Fc receptor (FcRn). In some embodiments, a mutation that increases binding to FcRn is selected from the mutations provided in Table 7. In some embodiments, the mutation is mutation of asparagine 434 to histidine (N434H). In some embodiments, an N434H mutated Fc domain comprises an N214H mutation of SEQ ID NO: 164 or 165. In some embodiments, the mutation is mutation of valine 308 to proline (V308P). In some embodiments, an H435A mutated Fc domain comprises an H215A mutation of SEQ ID NO: 164 or 165. In some embodiments, the mutation attenuates binding to FcRN. In some embodiments, the mutation that attenuates binding is mutation of histidine 435 to alanine (H435A). In some embodiments, an H435A mutated Fc domain comprises an H215A mutation of SEQ ID NO: 164 or 165. In some embodiments, the mutation that increases binding to FcRn is a plurality of mutations. In some embodiments, the plurality comprises or consists of mutation of methionine 252 to tyrosine (M252Y), mutations of serine 234 to threonine and mutation of threonine 256 to glutamic acid (T256E) (also termed YTE). In some embodiments, an M252Y/S254T/T256E mutated Fc domain comprises an M32Y, S34T and T35E mutation of SEQ ID NO: 164 or 165. In some embodiments, the plurality comprises or consists of mutation of methionine 428 to leucine (M428L) and mutation of asparagine 434 to serine (N434S) (also termed LS). In some embodiments, an M428L/N434S mutated Fc domain comprises an M208L and N214S mutation of SEQ ID NO: 164 or 165. In some embodiments, the plurality comprises or consists of M428L and mutation of asparagine 434 to alanine (N434A) (also termed LA). In some embodiments, an M428L/N434A mutated Fc domain comprises an M208L and N214A mutation of SEQ ID NO: 164 or 165. In some embodiments, the plurality comprises or consists of mutation of threonine 250 to glutamine (T250Q) and mutation of methionine 428 to leucine (M428L) (also termed QL). In some embodiments, an T250Q/M428L mutated Fc domain comprises an T30Q and M208L mutation of SEQ ID NO: 164 or 165. In some embodiments, the plurality comprises or consists of mutation of histidine 433 to lysine (H433K) and mutation of asparagine 434 to phenylalanine (N434F). In some embodiments, an H433K/N434F mutated Fc domain comprises an H213K and N214F mutation of SEQ ID NO: 164 or 165. In some embodiments, the plurality comprises or consists of M252Y, S254T, T256E, H433K and N434F. In some embodiments, an M252Y/S254T/T256E/H433K/N434F mutated Fc domain comprises an M32Y, S34T, T35E, H213K and N214F mutation of SEQ ID NO: 164 or 165. In some embodiments, the plurality comprises or consists of mutation of threonine 307 to alanine (T307A), mutation of glutamic acid 380 to alanine (E380A) and mutation of asparagine 434 to alanine (N434A). In some embodiments, an T307A/E380A/N434A mutated Fc domain comprises an T87A, E160A and N214A mutation of SEQ ID NO: 164 or 165. In some embodiments, the plurality comprises or consists of mutation of methionine 252 to tyrosine (M252Y), mutation of valine 308 to protein (V308P) and mutation of asparagine 343 to tyrosine (N343Y). In some embodiments, an M252Y/V308P/N343Y mutated Fc domain comprises an M32Y, V88P and N123Y mutation of SEQ ID NO: 164 or 165. In some embodiments, the plurality comprises or consists of M252Y, mutation of valine 308 to proline (V308P) and mutation of asparagine 434 to tyrosine (N434Y). In some embodiments, an M252Y/V308P/N434Y mutated Fc domain comprises an M32Y, V88P and N214Y mutation of SEQ ID NO: 164 or 165. In some embodiments, the plurality comprises or consists of mutation of histidine 258 to aspartic acid (H258D), mutation of threonine 307 to glutamine (T307Q) and mutation of alanine 378 to valine (A378V). In some embodiments, an H258D/T307Q/A378V mutated Fc domain comprises an H38D, T87Q and A158V mutation of SEQ ID NO: 164 or 165. In some embodiments, the plurality comprises or consists of mutation of leucine 309 to aspartic acid (L309D), mutation of glutamine 311 to histidine (Q311H) and mutation of asparagine 434 to serine (N434S). In some embodiments, an L309D/Q311H/N434S mutated Fc domain comprises an L89D, Q91H and N214A mutation of SEQ ID NO: 164 or 165. In some embodiments, the plurality that attenuates binding comprises or consists of mutation of isoleucine 253 to alanine (1253A), H435A and mutation of histidine 436 to alanine (H436A). In some embodiments, an I253A/H435A/H436A mutated Fc domain comprises an I33A, H215A and H216A mutation of SEQ ID NO: 164 or 165. In some embodiments, the plurality that attenuates binding comprises or consists of I253A, mutation of histidine 310 to alanine (H310A) and H435A. In some embodiments, an I253A/H310A/H435A mutated Fc domain comprises an I33A, H90A and H215A mutation of SEQ ID NO: 164 or 165.

TABLE 7
Mutations influencing FcRn binding

| Mutation | Description |
|---|---|
| M252Y/S254T/T256E (YTE) | Increased in 10-fold FcRn binding at pH |
| | 6.0 in comparison to WT hIgG1 |
| | Increase half-life |
| M428L/N434S (LS) | Increased in 11-fold FcRn binding at pH |
| | 6.0 in comparison to WT hIgG1, |
| | Increase half-life |
| M428L/N434A (LA) | Increase binding affinity to FcRn under |
| | pH 6.0 and prolong serum half-life |
| T250Q/M428L (QL) | Increase binding affinity to FcRn under |
| | pH 6.0 and prolong serum half-life |
| N434H | Prolong the half-life by enhancing |
| | FcRn binding |
| M252Y/S254T/T256E (YTE) + | Increase binding affinity to FcRn under |
| H433K/N434F | pH 6.0 and 7.4 |
| H433K/N434F | Increase binding affinity to FcRn under |
| | pH 6.0 and 7.4 |
| T307A/E380A/N434A | Increase binding affinity to FcRn under |
| | pH 6.0 and 7.4; |
| | Prolong the half-life by enhancing |
| | FcRn binding |
| M252Y/V308P/N343Y | Increase binding affinity to FcRn under |
| | pH 6.0 and 7.4; |
| | Prolong the half-life by enhancing FcRn |
| | binding |
| V308P | Increase binding affinity to FcRn under |
| | pH 6.0 and prolong serum half-life |
| M252Y/V308P/N434Y | Increase binding affinity to FcRn under |
| | pH 6.0 and 7.4 |
| H258D/T307Q/A378V | Increase binding affinity to FcRn under |
| | pH 6.0 and prolong serum half-life |
| L309D/Q311H/N434S | Increase binding affinity to FcRn under |
| | pH 6.0 and prolong serum half-life |
| H435A | Attenuate binding to FcRn at pH = 6.0 |
| I253A, H435A, H436A | Disable binding to FcRn |
| I253A/H310A/H435A | Reduced FcRn binding |

In some embodiments, the mutation is a mutation that decreases binding to an Fc receptor. In some embodiments, the Fc receptor is FcγR. In some embodiments, FcγR is FcγRI. In some embodiments, the mutation is a mutation that decreases binding to Clq. In some embodiments, a mutation that decreases binding to Fc receptor decreases ADCC. In some embodiments, the mutation is mutation of N297. As N-glycans are linked to N297 its mutation abrogates the glycosylation of this residue. In some embodiments, mutation of N297 is mutation to alanine (N297A). In some embodiments, mutation of N297 is mutation to glutamine (N297Q). In some embodiments, mutation of N297 is mutation to glycine (N297G). In some embodiments, an N297A mutated CH2 domain comprises an N59A mutation of SEQ ID NO: 139. In some embodiments, an N297A mutated Fc domain comprises an N77A mutation of SEQ ID NO: 164 or 165. In some embodiments, an N297Q mutated CH2 domain comprises an N59Q mutation of SEQ ID NO: 139. In some embodiments, an N297Q mutated Fc domain comprises an N77Q mutation of SEQ ID NO: 164 or 165. In some embodiments, an N297G mutated CH2 domain comprises an N59G mutation of SEQ ID NO: 139. In some embodiments, an N297G mutated Fc domain comprises an N77G mutation of SEQ ID NO: 164 or 165. In some embodiments, the mutation is a plurality of mutations that decrease binding to an Fc receptor. In some embodiments, the plurality comprises or consists of glycine 236 to arginine (G236R) and mutation of leucine 328 to arginine (L328R). In some embodiments, an G236R/L328R mutated Fc comprises a hinge domain comprising a G21R mutation of SEQ ID NO: 125 and a CH2 domain comprising a L90R mutation of SEQ ID NO: 139. In some embodiments, a G236R/L328R mutated Fc domain comprises an G16R and L108R mutation of SEQ ID NO: 164 or 165. In some embodiments, the plurality comprises or consists of serine 298 to glycine (S298G) and mutation of threonine 299 to alanine (T299A). In some embodiments, an S298G/T299A mutated CH2 domain comprises a S60G and T61A mutation of SEQ ID NO: 139. In some embodiments, a S298G/T299A mutated Fc domain comprises an S78G and T79A mutation of SEQ ID NO: 164 or 165. In some embodiments, the plurality comprises or consists of leucine 234 to phenylalanine (L234F), leucine 235 to glutamic acid (L235E) and mutation of aspartic acid 265 to arginine (D265A). In some embodiments, an L234F/L235E/D265A mutated Fc comprises a hinge domain comprising a L19F and L20E mutation of SEQ ID NO: 125 and a CH2 domain comprising a D27A mutation of SEQ ID NO: 139. In some embodiments, a L234F/L235E/D265A mutated Fc domain comprises an L14F, L15E and D45A mutation of SEQ ID NO: 164 or 165. In some embodiments, the plurality comprises or consists of leucine 234 to alanine (L234A), leucine 235 to alanine (L235A) and mutation of proline 329 to glycine (P329G). In some embodiments, an L234A/L235A/P329G mutated Fc comprises a hinge domain comprising a L19A and L20A mutation of SEQ ID NO: 125 and a CH2 domain comprising a P91G mutation of SEQ ID NO: 139. In some embodiments, a L234A/L235A/P329G mutated Fc domain comprises an L14A, L15A and P109G mutation of SEQ ID NO: 164 or 165. In some embodiments, the plurality comprises or consists of L234F, L235E and mutation of proline 331 to serine (P331S). In some embodiments, an L234F/L235E/P331S mutated Fc comprises a hinge domain comprising a L19F and L20E mutation of SEQ ID NO: 125 and a CH2 domain comprising a P93S mutation of SEQ ID NO: 139. In some embodiments, a L234F/L235E/P331S mutated Fc domain comprises an L14F, L15E and P111S mutation of SEQ ID NO: 164 or 165. In some embodiments, the plurality comprises or consists of leucine 235 to alanine (L235A), glycine 237 to alanine (G237A) and mutation of glutamic acid 318 to alanine (E318A). In some embodiments, an L235A/G237A/E318A mutated Fc comprises a hinge domain comprising a L20A and G22A mutation of SEQ ID NO: 125 and a CH2 domain comprising a E80A mutation of SEQ ID NO: 139. In some embodiments, a L235A/G237A/E318A mutated Fc domain comprises an L15A, G17A and E98A mutation of SEQ ID NO: 164 or 165.

In some embodiments, the Fc is modified to decrease binding to Fc receptor. In some embodiments, the modification is removal of glycosylation. In some embodiments, Fc glycosylation is removed enzymatically. In some embodiments, enzymatic de-glycosylation is performed with a deglycosylase. In some embodiments, enzymatic de-glycosylation is performed with a cleavase that cleaves sugars. Examples of enzymes for de-glycosylation include but are not limited to Peptide-N-Glycosidase F (PNGase) and Endoglycosidase H (Endo H). Kits for de-glycosylation are also commercially available.

In some embodiments, the mutation is a mutation that increases binding to an Fc receptor. In some embodiments, the Fc receptor is selected from FcγRI, FcγRIIA, FcγRIIIA, and FcγRIIIB. In some embodiments, the Fc receptor is FcγRI. In some embodiments, the mutation is mutation of serine 267 to glutamic acid (S267E). In some embodiments, an S267E mutated CH2 domain comprises an S29E mutation of SEQ ID NO: 139. In some embodiments, an S267E mutated Fc domain comprises an S47E mutation of SEQ ID NO: 164 or 165. In some embodiments, the mutation is mutations of proline 238 to aspartic acid (P238D). In some embodiments, a P238D mutated hinge domain comprises an P23D mutation of SEQ ID NO: 125. In some embodiments, a P238D mutated Fc domain comprises an P18D mutation of SEQ ID NO: 164 or 165. In some embodiments, the mutation is a plurality of mutations that increase binding to an Fc receptor. In some embodiments, the plurality comprises or consists of S267E and mutation of leucine 328 to phenylalanine (L328F) (also termed SELF). In some embodiments, an S267E/L328F mutated CH2 domain comprises an S29E and L90F mutation of SEQ ID NO: 139. In some embodiments, an S267E/L328F mutated Fc domain comprises an S47E and L108F mutation of SEQ ID NO: 164 or 165. In some embodiments, the plurality comprises or consists of S267E and mutation of histidine 268 to phenylalanine (H268F) and mutation of serine 324 to threonine (S324T) (also termed EFT). In some embodiments, an S267E/H268F/S324T mutated CH2 domain comprises an S29E, H30F and S86T mutation of SEQ ID NO: 139. In some embodiments, an S267E/H268F/S324T mutated Fc domain comprises an S47E, H48F and S104T mutation of SEQ ID NO: 164 or 165. In some embodiments, the plurality comprises or consists of mutation of glycine 237 to aspartic acid (G237D), P238D, proline 271 to glycine (P271G) and mutation of alanine 330 to arginine (A330R) (also termed V9). In some embodiments, a G237D/P238D/P271G/A330R mutated polypeptide comprises a mutated hinge domain comprising a G22D and P23D mutation of SEQ ID NO: 125 and a mutated CH2 domain comprising a P33G and A92R mutation of SEQ ID NO: 139. In some embodiments, a G237D/P238D/P271G/A330R mutated Fc domain comprises a G17D, P18D, P51G and A110R mutation of SEQ ID NO: 164 or 165. In some embodiments, the plurality comprises or consists of mutation of G237D, P238D, histidine 268 to aspartic acid (H268D), P271G and A330R (also termed V11). In some embodiments, a G237D/P238D/H268D/P271G/A330R mutated polypeptide comprises a mutated hinge domain comprising a G22D and P23D mutation of SEQ ID NO: 125 and a mutated CH2 domain comprising a H30D, P33G and A92R mutation of SEQ ID NO: 139. In some embodiments, a G237D/P238D/H268D/P271G/A330R mutated Fc domain comprises a G17D, P18D, H48D, P51G and A110R mutation of SEQ ID NO: 164 or 165. In some embodiments, the plurality comprises or consists of mutation of glutamic acid 233 to aspartic acid (E233D), G237D, P238D, H268D, P271G and A330R (also termed V12). In some embodiments, a E233D/G237D/P238D/H268D/P271G/A330R mutated polypeptide comprises a mutated hinge domain comprising a E18D, G22D and P23D mutation of SEQ ID NO: 125 and a mutated CH2 domain comprising a H30D, P33G and A92R mutation of SEQ ID NO: 139. In some embodiments, a E233D/G237D/P238D/H268D/P271G/A330R mutated Fc domain comprises a E13D, G17D, P18D, H48D, P51G and A110R mutation of SEQ ID NO: 164 or 165.

The S267E mutation was found to enhance affinity toward the inhibitory FcγRIIB and also toward the activating FcγRIIa. The SELF mutations in hIgG1 resulted in a substantial 430-fold increase in the binding toward FcγRIIB, with minimal alterations in binding to FcγRI and FcγRIIA-H131 in comparison to human WT IgG1. The EFT mutation was found to increase FcγRIIB binding by 18-fold in comparison to human WT IgG1. EFT also increased CDC, ADCC and antibody-dependent cellular phagocytosis (ADCP) activity via the enhancement of Clq and activator FcG receptors binding. In some embodiments, a mutation that increases ADCC is the EFT plurality of mutations. P238D demonstrated enhanced binding to FcγRIIB with about 4.3-fold increased affinity in comparison to WT human IgG1. P238D also significantly reduces the binding toward all other activating Fcg receptors. V9 significantly enhanced the affinity of antibodies toward hFcγRIIB, by approximately a 32-fold change in comparison to WT IgG1. V9 also was found to reduce the affinity toward hFcγRIIA R131 allele by about 3-fold in comparison to WT IgG1. V11 was found to significantly enhance the affinity of antibodies for hFcγRIIB by approximately 96-fold, while reducing the affinity toward hFcγRIIA R131 by about 3-fold in comparison to human WT IgG1. V12 demonstrated significant enhancement of binding toward FcγRIIB, with 217-fold change in comparison to human WT IgG1. V12 mutations also show no detectable binding toward FcγRIIIA allotypes, reduced FcγRI binding (0.061-fold change relative to wt IgG1) and FcγRIIA-H131 (0.068-fold change relative to wt IgG1). It should be noted that V12 slightly improve the binding toward FcγRIIA-R131, with a 2-fold binding increase in compared to WT hIgG1.

Mutations that produce the above recited functions are well known in the art and any such mutation can be used. Examples of such mutations can be found at least in K. O. Saunders, 2019, “Conceptual approaches to modulating antibody effector functions and circulation half-life”, Front, Immunol., 2019 Jun. 7; 10:1296, herein incorporated by reference in its entirety. Table 1 of Saunders provides Fc modifications that enhance antibody effector function. Table 2 of Saunders provides Fc modifications that improve antibody circulation half-life. Table 3 of Saunders provides Fc modifications that inhibit antibody effector function. It will be understood by a skilled artisan that parallel mutation can also be performed in the IgG3 heavy chain or the heavy chains of non-human IgGs. It will be understood that the number given herein is in reference to a full-length IgG including the variable domains. The numbers can be shifted to correspond to the positions of these amino acids within just the Fc portion of the IgG.

In some embodiments, the mutation increases effector function. In some embodiments, the mutation increases ADCC. In some embodiments, the mutation is not a mutation that increases CDC. In some embodiments, the mutation increases ADCC and not CDC. It will be understood by a skilled artisan that while the unmodified Fc is not sufficiently cytotoxic to overcome the booster effect produced by the molecules of the invention, an Fc comprising a mutation that increases ADCC is. In some embodiments, effector function comprises ADCC. In some embodiments, effector function comprises ADCC and not CDC. In some embodiments, increased effector function comprises increased cytotoxicity. In some embodiments, the Fc is from IgG1 or IgG3 and the mutation increases effector function. In some embodiments, the Fc is from IgG1 and comprises at least one mutation that increases effector function. Mutations that increase effector function are well known in the art and any such mutation can be used. Examples of such mutations can be found in Liu, 2020, “Fc-engineering for modulated effector functions-improving antibodies for cancer treatment” Antibodies (Basel), 2020 December; 9 (4): 64, herein incorporated by reference in its entirety.

In some embodiments, a mutation that increases ADCC is a plurality of mutations that increase ADCC. In some embodiments, the plurality of mutations comprises mutation of leucine 235 to valine (L235V), phenylalanine 243 to leucine (F243L), arginine 292 to proline (R292P), tyrosine 300 to leucine (Y300L) and proline 296 to leucine (P396L) within human IgG1. In some embodiments, the plurality of mutations comprises mutation of leucine 15 to valine (L15V), phenylalanine 23 to leucine (F23L), arginine 72 to proline (R72P), tyrosine 80 to leucine (Y80L) and proline 176 to leucine (P176L) within SEQ ID NO: 164. In some embodiments, the plurality of mutations comprises mutation of serine 239 to aspartic acid (S239D) and isoleucine 332 to glutamic acid (1332E) within human IgG1. In some embodiments, the plurality of mutations comprises mutation of serine 19 to aspartic acid (S19D) and isoleucine 112 to glutamic acid (1112E) within SEQ ID NO: 164. In some embodiments, the S239D/1332E mutations also increase ADCP. In some embodiments, the plurality of mutations comprises mutation of serine 239 to aspartic acid (S239D), alanine 330 to leucine (A330L) and isoleucine 332 to glutamic acid (1332E) within human IgG1. In some embodiments, the plurality of mutations comprises mutation of serine 19 to aspartic acid (S19D), alanine 110 to leucine (A110L) and isoleucine 112 to glutamic acid (I112E) within SEQ ID NO: 164. In some embodiments, the S239D/A330L/1332E mutations also increase ADCP. In some embodiments, the plurality of mutations comprises mutation of glycine 236 to alanine (G236A), alanine 330 to leucine (A330L) and isoleucine 332 to glutamic acid (1332E) within human IgG1. In some embodiments, the plurality of mutations comprises mutation of glycine 16 to alanine (G16A), alanine 110 to leucine (A110L) and isoleucine 112 to glutamic acid (1112E) within SEQ ID NO: 164. In some embodiments, the plurality of mutations comprises mutation of glycine 236 to alanine (G236A), serine 267 to glutamic acid (S267E), histidine 268 for phenylamine (H268F), serine 324 to threonine (S324T) and isoleucine 332 to glutamic acid (1332E) within human IgG1. In some embodiments, the plurality of mutations comprises mutation of glycine 16 to alanine (G16A), serine 47 to glutamic acid (S47E), histidine 48 for phenylamine (H48F), serine 104 to threonine (S104T) and isoleucine 112 to glutamic acid (1112E) within SEQ ID NO: 164. In some embodiments, the plurality of mutations comprises mutation of serine 298 to alanine (S298A), glutamic acid 333 to alanine (E333A), and lysine 334 to alanine (K334A) within human IgG1. In some embodiments, the plurality of mutations comprises mutation of mutation of serine 78 to alanine (S78A), glutamic acid 113 to alanine (E113A), and lysine 114 to alanine (K114A) within SEQ ID NO: 164. In some embodiments, the plurality of mutations comprises mutation of proline 247 to isoleucine (P2471), and alanine 339 to glutamine (A339Q) within human IgG1. In some embodiments, the plurality of mutations comprises mutation of mutation of proline 27 to isoleucine (P27I), and alanine 119 to glutamine (A119Q) within SEQ ID NO: 164. In some embodiments, the plurality of mutations comprises mutation of glycine 236 to alanine (G236A), serine 239 to aspartic acid (S239D) and isoleucine 332 to glutamic acid (1332E) within human IgG1. In some embodiments, the plurality of mutations comprises mutation of glycine 16 to alanine (G16A), serine 19 to aspartic acid (S19D) and isoleucine 112 to glutamic acid (1112E) within SEQ ID NO: 164. In some embodiments, the G236A/S239D/1332E mutations also increase ADCP. In some embodiments, the plurality of mutations comprises mutation of lysine 234 to tyrosine (L234Y), lysine 235 to glutamine (L235Q), glycine 236 to tryptophan (G236W), serine 239 to methionine (S239M), histidine 268 to aspartic acid (H268D), aspartic acid 270 to glutamic acid (D270E) and serine 298 to alanine (S298A) within a first heavy chain of human IgG1 and mutation of aspartic acid 270 to glutamic acid (D270E), lysine 326 to aspartic acid (K26D), alanine 330 to methionine (A330M) and lysine 334 to glutamic acid (K334E) within the second heavy chain of IgG1. In some embodiments, the plurality of mutations comprises mutation of mutation of lysine 14 to tyrosine (L14Y), lysine 15 to glutamine (L15Q), glycine 16 to tryptophan (G16W), serine 19 to methionine (S19M), histidine 48 to aspartic acid (H48D), aspartic acid 50 to glutamic acid (D50E) and serine 78 to alanine (S78A) within a first chain of SEQ ID NO: 164 and mutation of aspartic acid 50 to glutamic acid (D50E), lysine 326 to aspartic acid (K106D), alanine 110 to methionine (A110M) and lysine 114 to glutamic acid (K114E) within a second chain of SEQ ID NO: 164. It will be understood that all of the above recited mutations given with respect to SEQ ID NO: 164 also apply to SEQ ID NO: 165. Indeed, they also apply to SEQ ID NO: 171 and SEQ ID NO: 155, but all numbering given hereinabove must be increased by 5 for these sequences.

In some embodiments, the Fc domain with increased ADCC comprises L15V/F23L/R72P/Y80L/P176L mutations within the Fc domain. In some embodiments, the Fc domain is selected from SEQ ID NO: 164 and SEQ ID NO: 165. In some embodiments, the Fc domain with increased ADCC comprises EPKSCDKTHTCPPCPAPELVGGPSVFLLPPKPKDTLMISRTPEVTCVVVDVSHEDPE VKFNWYVDGVEVHNAKTKPPEEQYNSTLRVVSVLTVLHQDWLNGKEYKCKVSN KALPAPIEKTISKAKGQPREPQVYTLPPSRDELTKNQVSLTCLVKGFYPSDIA VEWE SNGQPENNYKTTPLVLDSDGSFFLYSKLTVDKSRWQQGNVFSCSVMHEALHNHY TQKSLSLSPGK (SEQ ID NO: 166). In some embodiments, the Fc domain with increased ADCC consists of SEQ ID NO: 166. In some embodiments, the Fc comprising the L15V/F23L/R72P/Y80L/P176L mutations is SEQ ID NO: 166. In some embodiments, the Fc domain with increased ADCC is at least 75, 80, 85, 90, 92, 95, 97 or 99% identical to SEQ ID NO: 166 and comprises L15V/F23L/R72P/Y80L/P176L mutations.

In some embodiments, the Fc domain with increased ADCC comprises S19D/A110L/1112E mutations within the Fc domain. In some embodiments, the Fc domain is selected from SEQ ID NO: 164 and SEQ ID NO: 165. In some embodiments, the Fc domain with increased ADCC comprises EPKSCDKTHTCPPCPAPELLGGPDVFLFPPKPKDTLMISRTPEVTCVVVDVSHEDPE VKFNWYVDGVEVHNAKTKPREEQYNSTYRVVSVLTVLHQDWLNGKEYKCKVSN KALPLPEEKTISKAKGQPREPQVYTLPPSREEMTKNQVSLTCLVKGFYPSDIAVEW ESNGQPENNYKTTPPVLDSDGSFFLYSKLTVDKSRWQQGNVFSCSVMHEALHNH YTQKSLSLSPGK (SEQ ID NO: 167). In some embodiments, the Fc domain with increased ADCC consists of SEQ ID NO: 167. In some embodiments, the Fc comprising the S19D/A110L/1112E mutations is SEQ ID NO: 167. In some embodiments, the Fc domain with increased ADCC is at least 75, 80, 85, 90, 92, 95, 97 or 99% identical to SEQ ID NO: 167 and comprises S19D/A110L/1112E mutations.

In some embodiments, the Fc domain with increased CDC comprises G16A/S47E/H48F/S104T/1112E mutations within the Fc domain. In some embodiments, the Fc domain is selected from SEQ ID NO: 164 and SEQ ID NO: 165. In some embodiments, the Fc domain with increased CDC comprises EPKSCDKTHTCPPCPAPELLAGPSVFLFPPKPKDTLMISRTPEVTCVVVDVEFEDPE VKFNWYVDGVEVHNAKTKPREEQYNSTYRVVSVLTVLHQDWLNGKEYKCKVT NKALPAPEEKTISKAKGQPREPQVYTLPPSREEMTKNQVSLTCLVKGFYPSDIAVE WESNGQPENNYKTTPPVLDSDGSFFLYSKLTVDKSRWQQGNVFSCSVMHEALHN HYTQKSLSLSPGK (SEQ ID NO: 168). In some embodiments, the Fc domain with increased CDC consists of SEQ ID NO: 168. In some embodiments, the Fc comprising the G16A/S47E/H48F/S104T/1112E mutations is SEQ ID NO: 168. In some embodiments, the Fc domain with increased CDC is at least 75, 80, 85, 90, 92, 95, 97 or 99% identical to SEQ ID NO: 168 and comprises G16A/S47E/H48F/S104T/1112E mutations.

In some embodiments, the Fc domain with increased ADCC comprises G16A/A110L/1112E mutations within the Fc domain. In some embodiments, the Fc domain is selected from SEQ ID NO: 164 and SEQ ID NO: 165. In some embodiments, the Fc domain with increased ADCC comprises EPKSCDKTHTCPPCPAPELLAGPSVFLFPPKPKDTLMISRTPEVTCVVVDVSHEDPE VKFNWYVDGVEVHNAKTKPREEQYNSTYRVVSVLTVLHQDWLNGKEYKCKVSN KALPLPEEKTISKAKGQPREPQVYTLPPSREEMTKNQVSLTCLVKGFYPSDIAVEW ESNGQPENNYKTTPPVLDSDGSFFLYSKLTVDKSRWQQGNVFSCSVMHEALHNH YTQKSLSLSPGK (SEQ ID NO: 169). In some embodiments, the Fc domain with increased ADCC consists of SEQ ID NO: 169. In some embodiments, the Fc comprising the G16A/A110L/1112E mutations is SEQ ID NO: 169. In some embodiments, the Fc domain with increased ADCC is at least 75, 80, 85, 90, 92, 95, 97 or 99% identical to SEQ ID NO: 169 and comprises G16A/A110L/1112E mutations.

In some embodiments, the effector domain is selected from SEQ ID NO: 166-169. In some embodiments, the effector domain comprises any one of SEQ ID NO: 166-169. In some embodiments, the effector domain consists of any one of SEQ ID NO: 166-169. In some embodiments, the effector domain is selected from SEQ ID NO: 166, 167 and 169. In some embodiments, the effector domain comprises any one of SEQ ID NO: 166, 167 and 169. In some embodiments, the effector domain consists of any one of SEQ ID NO: 166, 167 and 169. In some embodiments, the effector domain comprises at least 75, 80, 85, 90, 92, 95, 97 or 99% identity to any one of SEQ ID NO: 166, 167 and 169 and retains increased ADCC as compared to a control Fc domain. In some embodiments, the control Fc domain is an unmodified Fc domain. In some embodiments, unmodified Fc is an Fc found in nature. In some embodiments, unmodified Fc is a human Fc found in nature.

In some embodiments, the Fc is modified to increase ADCC. In some embodiments, the modification is removal of fucosylation. In some embodiments, Fc fucosylation is removed enzymatically. In some embodiments, the Fc is afucosylated. In some embodiments, the method comprises performing afucosylation of the molecule. In some embodiments, the molecules of the invention are produced in a cell line engineered to produce afucosylated molecules.

In some embodiments, the mutation increases CDC. In some embodiments, a plurality of mutations increases CDC. In some embodiments, the plurality of mutations comprises mutation of glycine 236 to alanine (G236A), serine 267 to glutamic acid (S267E), histidine 268 for phenylamine (H268F), serine 324 to threonine (S324T) and isoleucine 332 to glutamic acid (1332E) within human IgG1. In some embodiments, the plurality of mutations comprises mutation of glycine 16 to alanine (G16A), serine 47 to glutamic acid (S47E), histidine 48 for phenylamine (H48F), serine 104 to threonine (S104T) and isoleucine 112 to glutamic acid (I112E) within SEQ ID NO: 164. In some embodiments, the plurality of mutation comprises mutation of lysine 326 to tryptophan (K326W) and glutamic acid 333 to serine (E333S) within human IgG1. In some embodiments, the plurality of mutations comprises mutation of lysine 106 to tryptophan (K106W) and glutamic acid 113 to serine (E113S) within SEQ ID NO: 164. In some embodiments, the plurality of mutation comprises mutation of glutamic acid 345 to arginine (E345R), glutamic acid 430 to glycine (E430G) and serine 440 to tyrosine (S440Y) within human IgG1. In some embodiments, the plurality of mutations comprises mutation of glutamic acid 125 to arginine (E125R), glutamic acid 210 to glycine (E210G) and serine 220 to tyrosine (S220Y) within SEQ ID NO: 164. It will be understood that all of the above recited mutations given with respect to SEQ ID NO: 164 also apply to SEQ ID NO: 165. Indeed, they also apply to SEQ ID NO: 171 and SEQ ID NO: 155, but all numbering given hereinabove must be increased by 5 for these sequences.

In some embodiments, the effector moiety is a drug. In some embodiments, the protein is a DSG ECD drug conjugate. In some embodiments, the protein is an DSG ECD fragment drug conjugate. In some embodiments, the protein is a DSG-Fc drug conjugate. In some embodiments, the complex is a DSG ECD drug conjugate. In some embodiments, the complex is a DSG-Fc drug conjugate. In some embodiments, the effector moiety is cytotoxic. In some embodiments, the effector moiety is radioactive. In some embodiments, the effector moiety is a radioactive moiety. In some embodiments, effector moiety is a radioactive label. In some embodiments, the effector moiety is a chemotherapeutic. In some embodiments, the effector moiety is not a chemotherapeutic. In some embodiments, the effector moiety is toxic to a cell that is not replicating. In some embodiments, toxic is lethal. In some embodiments, the effector moiety is sufficient to kill a cell. Drug conjugation, and particularly drug conjugation to an antibody backbone, are well known in the art and any method of conjugation may be used.

In some embodiments, the effector moiety is an amatoxin. In some embodiments, the effector moiety is an amanitin. Amatoxins are a group of toxic compounds found in poisonous mushrooms. These are made up of eight amino acid residues arranged in a macrobicyclic motif and inhibit RNA polymerase. Amatoxins are also known as amanitins. In some embodiments, the amanitin is selected from alpha-amanitin, beta-amanitin, gamma-amanitin, epsilon-amanitin, amanullin, amanullinic acid, amaninamide, amanin and proamanullin. In some embodiments, the amanitin is alpha-amanitin. In some embodiments, the effector moiety is alpha-amanitin.

In some embodiments, the chemotherapeutic is an anthracycline. In some embodiments, the effector moiety is an anthracycline. Anthracyclines are a class of drugs extracted from streptomyces bacterium that intercalate into DNA and cause cytotoxicity primarily by inhibiting topoisomerase. Examples of anthracyclines include, but are not limited to doxorubicin, daunorubicin, epirubicin, nemorubicin, PNU-159682, ladirubicin and idarubicin. In some embodiments, the anthracycline is PNU-159682.

In some embodiments, the chemotherapeutic is an anthramycin-based dimer. In some embodiments, the anthramycin-based dimer is a pyrrolobenzodiazepine (PBD). In some embodiments, the chemotherapeutic is PBD. In some embodiments, the anthramycin-based dimer is an indolinobenzodiazepine dimers (IGN). In some embodiments, the chemotherapeutic is a pyrridinobenzodiazepine (PDD). In some embodiments, the anthramycin-based dimer is PDD. In some embodiments, the effector moiety is a PBD. In some embodiments, the effector moiety is a PDD. PBDs and PDDs are families of DNA minor-grove binding agents that inhibits DNA and RNA synthesis. In some embodiments, the PBD is a PBD dimer. Examples of PBDs and PDDs include, but are not limited to anthramycin, SJG-136, NS 694501 and FGX2-62. In some embodiments, the PBD is anthramycin. In some embodiments, the effector moiety is anthramycin. In some embodiments, anthramycin is anthramycin-methyl-ether (AME). In some embodiments, anthramycin is an anthramycin based dimer. In some embodiments, the PBD is tesirine (SG3249). In some embodiments, tesirine is SG3199. In some embodiments, the chemotherapeutic SG3249. In some embodiments, the chemotherapeutic is SG3199.

In some embodiments, the chemotherapeutic is a calicheamicin. In some embodiments, the effector moiety is a calicheamicin. Calicheamicins are a class of antibiotics derived from bacterium micromonospora echinospora that bind the DNA minor groove and cause strand scission. Examples of calicheamicins include but are not limited to calicheamicin gamma 1, esperamicin and ozogamicin.

In some embodiments, the chemotherapeutic is camptothecin or an analog thereof. In some embodiments, the effector moiety is camptothecin or an analog thereof. In some embodiments, the effector moiety is camptothecin. Examples of analogs of camptothecin include, but are not limited to exatecan, SN-38, and deruxtecan (Dxd). In some embodiments, the camptothecin analog is Dxd. In some embodiments, the chemotherapeutic is Dxd. In some embodiments, the effector moiety is Dxd.

In some embodiments, the chemotherapeutic is a duocarmycin. In some embodiments, the effector moiety is a duocarmycin. Duocarmycins are small molecules isolated from streptomyces bacteria that bind the DNA minor groove and alkylate adenine bases. Examples of duocarmycins include, but are not limited to duocarmycin A, duocarmycin B1, duocarmycin B2, duocarmycin C1, duocarmycin C2, duocarmycin D, duocarmycin SA, duocarmycin TM, duocarmycin MA and CC-1065.

In some embodiments, the chemotherapeutic is triptolide. In some embodiments, the effector moiety is triptolide.

In some embodiments, the effector moiety is a tubulin inhibitor. In some embodiments, the effector moiety is a maytansinoid. In some embodiments, the maytansinoid is a thiol containing maytansinoid. Mayttansinoids or maytansine are known to be tubulin inhibitors that inhibit the assembly of microtubules by binding tubulin att the rhizoxin binding site. In some embodiments, the maytansinoid is mertansine (DM-1). In some embodiments, mertansine is emtansine. In some embodiments, the tubulin inhibitor is an auristatin. In some embodiments, the auristatin is selected from Monomethyl auristatin E (MMAE) and Monomethyl auristatin F (MMAF). In some embodiments, the tubulin inhibitor is a tubulysin. In some embodiments, the tubulysin is tubulysin A. In some embodiments, the auristatin is MMAE. In some embodiments, the auristatin is MMAF. In some embodiments, the effector moiety is MMAE. In some embodiments, the effector moiety is MMAF.

In some embodiments, the effector moiety is a combination of moieties. In some embodiments, the effector moiety is a plurality of effector moieties. In some embodiments, the effector moiety is a combination of cytotoxic moieties. In some embodiments, the effector moiety comprises at least two cytotoxic moieties selected from the group consisting of: an amatoxin, an anthracycline, a pyrrolobenzodiazepine, a calicheamicin, a camptothecin, a duocarmycin, a triptolide, and a tubulin inhibitor. In some embodiments, the effector moiety comprises at least two cytotoxic moieties selected from the group consisting of: an amatoxin, an anthracycline, a pyrrolobenzodiazepine, a calicheamicin, a camptothecin, a duocarmycin, a triptolide, and a maytansinoid.

### Third and Fourth Chains

In some embodiments, the protein complex further comprises a third polypeptide chain. In some embodiments, the third polypeptide chain comprises a third fragment of a protein target of PV or PF autoantibodies. In some embodiments, the third fragment is different than the first fragment. In some embodiments, the third fragment is different than the second fragment. In some embodiments, the third fragment is the same as the first fragment. In some embodiments, the first fragment is the same as the second fragment. In some embodiments, the third fragment is the same as the first and second fragments. In some embodiments, the same as is the same sequence. In some embodiments, different is a different sequence.

In some embodiments, the third polypeptide further comprises a third dimerization domain. In some embodiments, the first polypeptide further comprises a fourth dimerization domain. In some embodiments, the third and fourth dimerization domains are capable of dimerizing to each other. In some embodiments, the third and fourth dimerization domains are configured to dimerizing to each other. In some embodiments, the third dimerization domain is not configured to dimerize to the first dimerization domain. In some embodiments, the third dimerization domain is not configured to dimerize to the second dimerization domain. In some embodiments, the fourth dimerization domain is not configured to dimerize to the first dimerization domain. In some embodiments, the fourth dimerization domain is not configured to dimerize to the second dimerization domain. In some embodiments, configured to dimerize is capable of dimerizing. In some embodiments, the third and fourth dimerization domains are different than the first and second dimerization domains. In some embodiments, the first and second dimerization domains are hinge domains and the third and fourth dimerization domains are CH1/CL domains. In some embodiments, the first and second dimerization domains are CH1/CL domains and the third and fourth dimerization domains are hinge domains.

In some embodiments, the protein complex further comprises a fourth polypeptide chain. In some embodiments, the fourth polypeptide chain comprises a fourth fragment of a protein target of PV or PF autoantibodies. In some embodiments, the fourth fragment is different than the first fragment. In some embodiments, the fourth fragment is different than the second fragment. In some embodiments, the fourth fragment is different than the third fragment. In some embodiments, the fourth fragment is the same as the first fragment. In some embodiments, the fourth fragment is the same as the second fragment. In some embodiments, the fourth fragment is the same as the third fragment. In some embodiments, the fourth fragment is the same as the first, second and third fragments. In some embodiments, the first, second, and third fragments are all the same. In some embodiments, the first, second, third and fourth fragments are all different. In some embodiments, the same as is the same sequence. In some embodiments, different is a different sequence. In some embodiments, different is from a different protein. In some embodiments, different is from the same protein but comprising a different sequence. In some embodiments, different is from the same protein but from a different region of the protein. In some embodiments, at least two of the first, second, third and fourth proteins are part of a single protein complex. In some embodiments, the protein complex is a complex in mammals. In some embodiments, the protein complex is a complex in humans.

In some embodiments, the fourth polypeptide further comprises a fifth dimerization domain. In some embodiments, the second polypeptide further comprises a sixth dimerization domain. In some embodiments, the fifth and sixth dimerization domains are capable of dimerizing to each other. In some embodiments, the fifth and sixth dimerization domains are configured to dimerizing to each other. In some embodiments, the fifth dimerization domain is not configured to dimerize to the first dimerization domain. In some embodiments, the fifth dimerization domain is not configured to dimerize to the second dimerization domain. In some embodiments, the fifth dimerization domain is not configured to dimerize to the third dimerization domain. In some embodiments, the fifth dimerization domain is not configured to dimerize to the fourth dimerization domain. In some embodiments, the sixth dimerization domain is not configured to dimerize to the first dimerization domain. In some embodiments, the sixth dimerization domain is not configured to dimerize to the second dimerization domain. In some embodiments, the sixth dimerization domain is not configured to dimerize to the third dimerization domain. In some embodiments, the sixth dimerization domain is not configured to dimerize to the fourth dimerization domain. In some embodiments, the fifth and sixth dimerization domains are different than the first and second dimerization domains. In some embodiments, the fifth and sixth dimerization domains are different than the third and fourth dimerization domains. In some embodiments, the first and second dimerization domains are hinge domains, the third and fourth dimerization domains are CH1/CL domains and the fifth and sixth dimerization domains are CH1/CL domains. In some embodiments, the first and second dimerization domains are CH1/CL domains, the third and fourth dimerization domains are hinge domains and the fifth and sixth dimerization domains are hinge domains. In some embodiments, the first polypeptide and second polypeptide do not both comprise a CH1 domain. In some embodiments, first polypeptide and second polypeptide both comprise a CH1 domain, first polypeptide and second polypeptide both comprise a CL domain. In some embodiments, first polypeptide and second polypeptide do not both comprise a CL domain. In some embodiments, the first polypeptide comprises a CH1 domain, and the second polypeptide comprises a CL domain. In some embodiments, the third polypeptide comprises a CL domain and the fourth polypeptide comprise a CH1 domain. In some embodiments, the first polypeptide comprises a CL domain and the second polypeptide comprises a CH1 domain. In some embodiments, the third polypeptide comprises a CH1 domain, and the fourth polypeptide comprise a CL domain.

In some embodiments, the third and fourth dimerization domains comprises mutations that permit dimerization of the third and fourth dimerization domains and inhibit dimerization of the third dimerization domain to the fifth, sixth or both dimerization domains. In some embodiments, the third and fourth dimerization domains comprises mutations that permit dimerization of the third and fourth dimerization domains and inhibit dimerization of the fourth dimerization domain to the fifth, sixth or both dimerization domains. In some embodiments, the fifth and sixth dimerization domains comprises mutations that permit dimerization of the fifth and sixth dimerization domains and inhibit dimerization of the fifth dimerization domain to the third, fourth or both dimerization domains. In some embodiments, the fifth and sixth dimerization domains comprises mutations that permit dimerization of the fifth and sixth dimerization domains and inhibit dimerization of the sixth dimerization domain to the third, sixth or both dimerization domains.

### Alternative Configurations

In some embodiments, the composition comprises a polypeptide chain comprising the fragment of a first protein target of PV or PF autoantibodies or an analog or derivative thereof and the fragment of a second protein target of PV or PF autoantibodies or an analog or derivative thereof. In some embodiments, the polypeptide chain is a single polypeptide chain. In some embodiments, the single chain comprises the fragment of the first protein and the fragment of the second protein. In some embodiments, the polypeptide chain further comprises a fragment of a third protein target of PV or PF autoantibodies or an analog or derivative thereof. In some embodiments, the polypeptide chain further comprises a fragment of a fourth protein target of PV or PF autoantibodies or an analog or derivative thereof. In some embodiments, the polypeptide chain further comprises an Fc region. In some embodiments, the polypeptide chain further comprises an effector moiety.

In some embodiments, the fragment of the first protein target of PV or PF autoantibodies or an analog or derivative thereof is separated from the fragment of the second protein target of PV or PF autoantibodies or an analog or derivative thereof by a linker. In some embodiments, the fragment of the third protein target of PV or PF autoantibodies or an analog or derivative thereof is separated from the fragment of the first or the second protein target of PV or PF autoantibodies or an analog or derivative thereof by a linker. In some embodiments, the fragment of the fourth protein target of PV or PF autoantibodies or an analog or derivative thereof is separated from the fragment of the first, the second or the third protein target of PV or PF autoantibodies or an analog or derivative thereof by a linker. In some embodiments, a fragment is separated from the Fc region by a linker. In some embodiments, the effector moiety is separated attached by a linker. In some embodiments, the effector moiety is separated by the fragment by a linker.

In some embodiments, the fragment and the dimerization domain are separated by a linker. In some embodiments, the dimerization domain and the Fc region are separated by a linker. In some embodiments, the fragment and the Fc region are separated by a linker. In some embodiments, the dimerization domain and the effector moiety are separated by a linker. In some embodiments, the linker is an amino acid linker. In some embodiments, the linker is a chemical linker. In some embodiments, the linker is a peptide linker. In some embodiments, the linker is a bond. In some embodiments, the bond is a peptide bond. In some embodiments, the bond is an amino acid bond. In some embodiments, the linker is a flexible linker. Linkers are well known in the art and any linker may be used.

In some embodiments, a linker is a chemical linker. In some embodiments, chemical linker is a polyethylene glycol (PEG) linker. In some embodiments, the PEG linker is a Gly3-PEG-azide linker. In some embodiments, the linker is a dibenzocyclooctyne group (DBCO) linker. In some embodiments, the DBCO linker is a DBCO-C6 linker. In some embodiments, the DBCO linker is a DBCO-Gly5-EDA linker. In some embodiments, the linker is dimethylethylenediamine (DMEDA) linker. In some embodiments, the linker is a N-dimethylethylenediamine (DMAE) linker. In some embodiments, the linker is a glutathione linker. In some embodiments, the linker is a CLICK linker. In some embodiments, the CLICK linker is a CLICK-DBCO linker. In some embodiments, the CLICK linker is a CLICK azide linker. In some embodiments, is a disulfide linker. In some embodiments, the linker is a thiol linker. In some embodiments, the linker isa azide linker. In some embodiments, the linker is a maleimide (Mal) linker. In some embodiments, the Mal linker is a maleimidocaproyl linker. In some embodiments, the Mal linker is a Mal-C6 linker. In some embodiments, the Mal linker is a Mal-Gly5-EDA linker. In some embodiments, the linker is a lysine linker. In some embodiments, the linker is an asparagine linker. In some embodiments, the linker is an acid-labile linker. In some embodiments, the linker is a cleavable linker. In some embodiments, cleavable is protease cleavable. In some embodiments, a cleavable linker is a glutathione cleavable linker. In some embodiments, the linker is a non-cleavable linker. Other examples of linkers include for example SPDB linkers, SMCC linkers, MCC linkers, and butanoic acid linkers. In some embodiments, the linker is a p-aminobenzyl (PAB) linker. In some embodiments, the linker is a p-aminocarbamate (PABC) linker. In some embodiments, the linker is a Maleimidocaproyl (mc) linker. In some embodiments, the linker comprises mc. In some embodiments, the linker is a Val-Cit-PAB linker. In some embodiments, the linker is a Val-Cit-PABC linker. In some embodiments, the linker is a Val-Cit-PAB-MMAE linker. In some embodiments, the linker is a mc-VC-PABC-MMAE linker. In some embodiments, the linker is a mc-MMAF linker. In some embodiments, the linker is a monomethyl auristatin E (MMAE) linker. Examples of peptide linkers include, but are not limited to Val-Cit-PAB linkers, Phe-Lys (Trt)-PAB linkers, and Ala-Ala-Asn-PAB linkers. In some embodiments, the linker is a mix of linkers. In some embodiments, the linker is a DBCO-PEG linker. In some embodiments, the linker is a PBCO-PEG-DMEDA linker. In some embodiments, the linker is a DBCO-PEG-VC-PAB-DMEDA linker. In some embodiments, VC in the linker is replaced with EVC. In some embodiments, VC in the linker is replaced with EVA. In some embodiments, the fragment and the dimerization domains are linked by a non-cleavable linker. In some embodiments, the fragment and the dimerization domains are linked by a cleavable linker. In some embodiments, the effector moiety is linked by a cleavable linker. In some embodiments, the effector moiety is linked by a non-cleavable linker.

In some embodiments, conjugated is linked. In some embodiments, conjugation is via a bond. In some embodiments, the conjugate is directly conjugated. In some embodiments, the conjugate is conjugated via a linker. In some embodiments, the effector moiety is conjugated by a linker.

In some embodiments, conjugating is conjugating of an amino acid linker, moiety or both and comprises extension of the amino acid sequence of a chain of the agent of the invention. It will be understood that a nucleic acid molecule encoding the agent of the invention can be modified to include the coding sequence for the linker, moiety or both and thus upon translation the full conjugate will be produced. In some embodiments, the conjugate is a fusion protein. Methods of linking and conjugating moieties are well known in the art and any such method may be used. In some embodiments, the method is a combination of at least two methods. In particular, methods of linking and conjugating to an IgG scaffold are also well known. Methods of linking/conjugating include, but are not limited to, native cysteine reduction (including native hinge reduction, also referred to herein as native cysteine conjugation), engineered cysteine reduction, disulphide bridging, lysine conjugation, and enzymatic conjugation. Examples of enzymatic conjugation include, but are not limited to: Click chemistry, sortase assisted-SMAC technology, transglutaminase addition of amine azide, and glycan remodeling.

Native cysteine conjugation was performed as follows. The CRD protein was reduced using TCEP and incubated at 37° C. for 90 minutes. Subsequently, DMA and the linker-payload were added, followed by a 2-hour incubation at room temperature. Finally, the conjugated materials were purified by Size-Exclusion Chromatography.

In some embodiments, the conjugation is site-specific conjugation. In some embodiments, the conjugation is not random conjugation. In some embodiments, the conjugation or linking is to the IgG backbone. In some embodiments, the conjugation or linking is not to a DSG1 or DSG3 fragment. In some embodiments, the conjugation or linking does not interfere with antibody binding to a DSG1 or DSG3 fragment. In some embodiments, the antibody is an autoantibody. In some embodiments, the conjugation or linking is to a dimerization domain. In some embodiments, the conjugation or linking is to the hinge region. In some embodiments, the conjugation or linking is to a CH2 region. In some embodiments, the conjugation or linking is to a CH3 region. In some embodiments, the conjugation or linking is to a CH1 region. In some embodiments, the conjugation or linking is to a CL region. In some embodiments, the linking or conjugating is to a native amino acid residue. In some embodiments, the linking or conjugating is to an engineered amino acid residue. In some embodiments, the residue is a cysteine. Examples of engineered cysteines include, but are not limited to A231C, S239C, N325C, L328C, D265C, and S442C of the heavy chain of IgG. In some embodiments, the residue is a lysine. In some embodiments, the residue is an asparagine. In some embodiments, glycan remodeling is used to link to an asparagine. In some embodiments, the asparagine is N297 of the heavy chain of IgG. In some embodiments, the residue is a glutamine. In some embodiments, N297 is converted, engineered, or mutated to glutamine (N297Q). In some embodiments, the glutamine is Q295 of the heavy chain of IgG. An example of an engineered glutamine includes but is not limited to Q297. The cites are provided with the Kabat numbering for IgG1 unless otherwise stated; corresponding mutations can be made in other IGs and specifically in other IgGs. In some embodiments, the linking or conjugating is to a C- or N-terminus of a chain of the agent of the invention. In some embodiments, the linking or conjugating is to a C-terminus. In some embodiments, the linking or conjugating is to an N-terminus. In some embodiments, the terminus is a terminus of the heavy chain. In some embodiments, the terminus is a terminus of the light chain. In some embodiments, the conjugation or linking is to a plurality of cites.

In some embodiments, the linker is of a sufficient length to inhibit steric hindrance between different sections of the chain. In some embodiments, the linker is of a sufficient length to inhibit steric hindrance between different sections of the conjugate. In some embodiments, the linker is of a sufficient length to allow binding of an antibody to the fragment without steric hindrance from another section of the chain. In some embodiments, the linker is of a sufficient length to allow binding of an antibody to the fragment without steric hindrance from another section of the conjugate. In some embodiments, the linker is of a sufficient length to allow binding of a cell to the fragment without steric hindrance from another section of the chain. In some embodiments, the linker is of a sufficient length to allow binding of a cell to the fragment without steric hindrance from another section of the conjugate. In some embodiments, the linker is at least 1, 2, 3, 4, 5, 6, 7, 8, 9, 10, 11, 12, 13, 14 or 15 amino acids in length. Each possibility represents a separate embodiment of the invention. In some embodiments, the linker is at least 1 amino acid in length. In some embodiments, the linker is at least 5 amino acids in length. In some embodiments, the linker is at least 10 amino acids in length. In some embodiments, the linker is at least 15 amino acids in length. In some embodiments, the linker is at most 10, 15, 20, 25, 30, 35, 40, 45, 50, 60, 70, 80, 90 or 100 amino acids in length. Each possibility represents a separate embodiment of the invention. In some embodiments, the linker is at most 10 amino acids in length. In some embodiments, the linker is at most 20 amino acids in length. In some embodiments, the linker is at most 50 amino acids in length. In some embodiments, the linker is at most 100 amino acids in length.

In some embodiments, the linker comprises GGGGS (SEQ ID NO: 152). In some embodiments, the linker consists of SEQ ID NO: 99. In some embodiments, the linker comprises (GGGGS) n wherein n is an integer. In some embodiments, the linker consists of (GGGGS) n wherein n is an integer. In some embodiments, the linker comprises GSAGSAAGSGEF (SEQ ID NO: 154). In some embodiments, the linker comprises or consists of (GGGS) nGS wherein n is an integer. In some embodiments, n is selected from 1, 2,3, 4 and 5. In some embodiments, n is 2. In some embodiments, n is 3. In some embodiments, n is 4. In some embodiments, n is 5. In some embodiments, the linker comprises or consists of GGGGSGGGGSGGGGSGGGGSGGGGS (SEQ ID NO: 153). In some embodiments, the linker consists of (GGGS) n wherein n is an integer.

In some embodiments, the linker is a rigid linker. In some embodiments, the rigid linker comprises EAAAK (SEQ ID NO: 170). In some embodiments, the rigid linker consists of SEQ ID NO: 170. In some embodiments, the rigid linker comprises (EAAAK) n where n is an integer. In some embodiments, the rigid linker consists of (EAAAK) n where n is an integer. In some embodiments, the rigid linker comprises (EAAAK) nGS where n is an integer. In some embodiments, the rigid linker consists of (EAAAK) nGS where n is an integer. In some embodiments, the rigid linker comprises (EAAAK) nGGS where n is an integer. In some embodiments, the rigid linker consists of (EAAAK) nGGS where n is an integer. In some embodiments, n is selected from 1, 2,3, 4, 5, 6, 7, 8, 9 and 10. Each possibility represents a separate embodiment of the invention. In some embodiments, n is 2. In some embodiments, n is 3. In some embodiments, n is 4. In some embodiments, n is 5.

In some embodiments, the dimerization domain is C-terminal to the fragment. In some embodiments, the fragment is C-terminal to the dimerization domain. In some embodiments, the Fc region is C-terminal to the fragment. In some embodiments, the fragment is C-terminal to the Fc region. In some embodiments, the dimerization domain is C-terminal to the Fc region. In some embodiments, the Fc region is C-terminal to the dimerization domain. In some embodiments, the dimerization domain is N-terminal to the fragment. In some embodiments, the fragment is N-terminal to the dimerization domain. In some embodiments, the Fc region is N-terminal to the fragment. In some embodiments, the fragment is N-terminal to the Fc region. In some embodiments, the dimerization domain is N-terminal to the Fc region. In some embodiments, the Fc region is N-terminal to the dimerization domain.

In some embodiments, the epitope spans at least two fragments. In some embodiments, the epitope spans the first and second fragments. In some embodiments, the epitope spans the first and third fragments. In some embodiments, the epitope spans the first and fourth fragments. In some embodiments, the epitope spans the second and third fragments. In some embodiments, the epitope spans the second and fourth fragments. In some embodiments, the epitope spans the third and fourth fragments. In some embodiments, the epitope spans two proteins. In some embodiments, the epitope spans two proteins in a protein complex. In some embodiments, the epitope spans three fragments. In some embodiments, the epitope spans three proteins. In some embodiments, the epitope spans four fragments. In some embodiments, the epitope spans four proteins. In some embodiments, the epitope is a complex epitope. In some embodiments, the epitope is a B cell receptor (BCR)-specific epitope.

In some embodiments, all fragments are from DSG1. In some embodiments, all fragments are from DSG3. In some embodiments, the complex comprises a fragment from DSG1 and a fragment from DSG3. In some embodiments, a fragment from DSG1 is mutated. In some embodiments, a fragment from DSG3 is mutated. In some embodiments, both a fragment from DSG1 and a fragment from DSG3 are mutated. In some embodiments, the DSG1 fragment and the DSG3 fragment bear the same or equivalent mutations. In some embodiments, the DSG1 and DSG3 fragment bear different and non-equivalent mutations. Equivalent mutations are mutations of the same amino acid, but with a slightly different numbered position due to differences in DSG1/3 sequence. In some embodiments, an equivalent mutation is a homologous mutation. In some embodiments, the complex comprises a DSG1 truncation. In some embodiments, the complex comprises a DSG3 truncation. In some embodiments, the complex comprises both a DSG1 and DSG3 mutation. In some embodiments, the DSG1 and DSG3 truncations are truncations of the same length. In some embodiments, the DSG1 and DSG3 truncations are truncations of different length. In some embodiments, the DSG1 and DSG3 truncations are truncations of the same domains.

In some embodiments, the first polypeptide comprises a fragment linked to EPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSN KALPAPIEKTISKAKGQPREPQVYTLPPSREEMTKNQVSLTCLVKGFYPSDIAVEW ESNGQPENNYKTTPPVLDSDGSFFLYSKLTVDKSRWQQGNVFSCSVMHEALHNH YTQKSLSLSPGK (SEQ ID NO: 155). In some embodiments, the second polypeptide comprises a fragment linked to SEQ ID NO: 155. In some embodiments, the first and second polypeptides both comprises a fragment linked to SEQ ID NO: 155.

In some embodiments, the first polypeptide comprises a fragment linked to EPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSN KALPAPIEKTISKAKGQPREPQVYTLPPSREEMTKNQVSLSCAVKGFYPSDIAVEW ESNGQPENNYKTTPPVLDSDGSFFLVSKLTVDKSRWQQGNVFSCSVMHEALHNH YTQKSLSLSPGK (SEQ ID NO: 156). In some embodiments, the second polypeptide comprises a fragment linked to SEQ ID NO: 156. In some embodiments, the first and second polypeptides both comprises a fragment linked to SEQ ID NO: 156.

In some embodiments, the first polypeptide comprises a fragment linked to EPKSCDKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYKCKVSN KALPAPIEKTISKAKGQPREPQVYTLPPSREEMTKNQVSLWCLVKGFYPSDIA VEW ESNGQPENNYKTTPPVLDSDGSFFLYSKLTVDKSRWQQGNVFSCSVMHEALHNH YTQKSLSLSPGK (SEQ ID NO: 157). In some embodiments, the second polypeptide comprises a fragment linked to SEQ ID NO: 157. In some embodiments, the first and second polypeptides both comprises a fragment linked to SEQ ID NO: 157.

In some embodiments, the first polypeptide comprises a fragment linked to SEQ ID NO: 137. In some embodiments, the second polypeptide comprises a fragment linked to SEQ ID NO: 137. In some embodiments, the third polypeptide comprises a fragment linked to SEQ ID NO: 137. In some embodiments, the fourth polypeptide comprises a fragment linked to SEQ ID NO: 137.

In some embodiments, the third polypeptide comprises a fragment linked to SEQ ID NO: 155. In some embodiments, the third polypeptide comprises a fragment linked to SEQ ID NO: 156. In some embodiments, the third polypeptide comprises a fragment linked to SEQ ID NO: 157. In some embodiments, the third polypeptide comprises a fragment linked to SEQ ID NO: 137. In some embodiments, the fourth polypeptide comprises a fragment linked to SEQ ID NO: 155. In some embodiments, the fourth polypeptide comprises a fragment linked to SEQ ID NO: 156. In some embodiments, the fourth polypeptide comprises a fragment linked to SEQ ID NO: 157. In some embodiments, the fourth polypeptide comprises a fragment linked to SEQ ID NO: 137.

In some embodiments, a polypeptide chain comprises or consists of an amino acid sequence selected from SEQ ID NO: 5-73. Each sequence represents a separate embodiment of the invention. In some embodiments, a polypeptide chain comprises or consists of an amino acid sequence selected from SEQ ID NO: 5-73 or an analog or derivative thereof with at least 85% identity. Each sequence represents a separate embodiment of the invention. In some embodiments, a polypeptide chain comprises or consists of an amino acid sequence selected from Table 2. In some embodiments, a polypeptide chain comprises or consists of an amino acid sequence selected from Tables 3. In some embodiments, a polypeptide chain comprises or consists of an amino acid sequence selected from Table 4. In some embodiments, a polypeptide chain comprises or consists of an amino acid sequence selected from Table 5. In some embodiments, the complex comprises or consists of two polypeptide chains selected from SEQ ID NO: 5-73. Each sequence represents a separate embodiment of the invention. In some embodiments, the complex comprises or consists of two polypeptide chains selected from SEQ ID NO: 5-73 or an analog or derivative thereof comprising at least 85% identity. Each sequence represents a separate embodiment of the invention. In some embodiments, the two chains are the same chain. In some embodiments, the two chains are different chains.

In some embodiments, the complex comprises or consists of CRD-290. In some embodiments, the complex comprises or consists of CRD-291. In some embodiments, the complex comprises or consists of CRD-292. In some embodiments, the complex comprises or consists of CRD-293. In some embodiments, the complex comprises or consists of CRD-294. In some embodiments, the complex comprises or consists of CRD-295. In some embodiments, the complex comprises or consists of CRD-296. In some embodiments, the complex comprises or consists of CRD-650. In some embodiments, the complex comprises or consists of CRD-652. In some embodiments, the complex comprises or consists of CRD-653. In some embodiments, the complex comprises or consists of CRD-654. In some embodiments, the complex comprises or consists of CRD-655. In some embodiments, the complex comprises or consists of CRD-656. In some embodiments, the complex comprises or consists of CRD-657. In some embodiments, the complex comprises or consists of CRD-658. In some embodiments, the complex comprises or consists of CRD-659. In some embodiments, the complex comprises or consists of CRD-660. In some embodiments, the complex comprises or consists of CRD-661. In some embodiments, the complex comprises or consists of CRD-662. In some embodiments, the complex comprises or consists of CRD-663. In some embodiments, the complex comprises or consists of CRD-664. In some embodiments, the complex comprises or consists of CRD-666. In some embodiments, the complex comprises or consists of CRD-667. In some embodiments, the complex comprises or consists of CRD-668. In some embodiments, the complex comprises or consists of CRD-669. In some embodiments, the complex comprises or consists of CRD-670. In some embodiments, the complex comprises or consists of CRD-671. In some embodiments, the complex comprises or consists of CRD-672. In some embodiments, the complex comprises or consists of CRD-673. In some embodiments, the complex comprises or consists of CRD-674. In some embodiments, the complex comprises or consists of CRD-675. In some embodiments, the complex comprises or consists of CRD-676. In some embodiments, the complex comprises or consists of CRD-677. In some embodiments, the complex comprises or consists of CRD-678. In some embodiments, the complex comprises or consists of CRD-679. In some embodiments, the complex comprises or consists of CRD-680. In some embodiments, the complex comprises or consists of CRD-681. In some embodiments, the complex comprises or consists of CRD-683. In some embodiments, the complex comprises or consists of CRD-684. In some embodiments, the complex comprises or consists of CRD-685. In some embodiments, the complex comprises or consists of CRD-686. In some embodiments, the complex comprises or consists of CRD-688. In some embodiments, the complex comprises or consists of CRD-689. In some embodiments, the complex comprises or consists of CRD-690. In some embodiments, the complex comprises or consists of CRD-691. In some embodiments, the complex comprises or consists of CRD-692. In some embodiments, the complex comprises or consists of CRD-693. In some embodiments, the complex comprises or consists of CRD-695. In some embodiments, the complex comprises or consists of CRD-696. In some embodiments, the complex comprises or consists of CRD-697. In some embodiments, the complex comprises or consists of CRD-698. In some embodiments, the complex comprises or consists of CRD-699. In some embodiments, the complex comprises or consists of CRD-700. In some embodiments, the complex comprises or consists of CRD-701. In some embodiments, the complex comprises or consists of CRD-702. In some embodiments, the complex comprises or consists of CRD-703. In some embodiments, the complex comprises or consists of CRD-923. In some embodiments, the complex comprises or consists of CRD-924. In some embodiments, the complex comprises or consists of CRD-925. In some embodiments, the complex comprises or consists of CRD-926. In some embodiments, a polypeptide chain comprises or consists of a sequence with at least 70% identity to a sequence provided herein. In some embodiments, a polypeptide chain comprises or consists of a sequence with at least 75% identity to a sequence provided herein. In some embodiments, a polypeptide chain comprises or consists of a sequence with at least 80% identity to a sequence provided herein. In some embodiments, a polypeptide chain comprises or consists of a sequence with at least 85% identity to a sequence provided herein. In some embodiments, a polypeptide chain comprises or consists of a sequence with at least 90% identity to a sequence provided herein. In some embodiments, a polypeptide chain comprises or consists of a sequence with at least 95% identity to a sequence provided herein. In some embodiments, a polypeptide chain comprises or consists of a sequence with at least 97% identity to a sequence provided herein. In some embodiments, a polypeptide chain comprises or consists of a sequence with at least 99% identity to a sequence provided herein.

### Pharmaceutical Compositions

By another aspect, there is provided a pharmaceutical composition comprising a protein or polypeptide of the invention.

By another aspect, there is provided a pharmaceutical composition comprising a protein complex of the invention.

By another aspect, there is provided a pharmaceutical composition comprises a composition of the invention.

In some embodiments, the pharmaceutical composition comprises a pharmaceutically acceptable carrier, excipient or adjuvant. As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition comprises a therapeutically effective amount of the protein complex of the invention. In some embodiments, the pharmaceutical composition comprises a therapeutically effective amount of the conjugate of the invention. The term “therapeutically effective amount” refers to an amount of a drug effective to treat a disease or disorder in a mammal. In some embodiments, a therapeutically effective amount is an amount effective, at dosages and for periods of time necessary, to achieve the desired therapeutic or prophylactic result. The exact dosage form and regimen would be determined by the physician according to the patient's condition. In some embodiments, an effective amount is an amount sufficient to treat at least one symptom of a disease. In some embodiments, the disease is PV. In some embodiments, the disease is PF. In some embodiments, the disease is PV or PF. In some embodiments, PV or PF is characterized by autoantibodies against the protein. In some embodiments, PV is characterized by autoantibody against DSG1 and/or DSG3. In some embodiments, PF is characterized by autoantibody against DSG1.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life. Treatment of PV or PF is well known in the art and may include any acceptable measure for assessing improvement of a PV or PF symptom. This may include, Rituximab, steroids, steroid-sparing immunosuppressants (such as azathioprine, mycophenolate and cyclophosphamide) dapsone, intravenous immunoglobulin (IVIG) and the like. Treatment may include improved quality of life, suppression of blister formation, reduction of autoantibodies and killing of autoreactive B cells.

In some embodiments, the pharmaceutical composition is formulated for systemic administration. In some embodiments, the pharmaceutical composition is formulated for administration to a subject. In some embodiments, the pharmaceutical composition is formulated for administration to a human. In some embodiments, the pharmaceutical composition is formulated for intravenous administration.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal. In some embodiments, the administering is intravenous administering. In some embodiments, the administering is topical administration. In some embodiments, the administering is selected from oral, intravenous, intramuscular, intraperitoneal, intertumoral, topical, or subdermal administration. In some embodiments, administering is administering to a site of disease.

The dosage administered will be dependent upon the age, health, and weight of the recipient, kind of concurrent treatment, if any, frequency of treatment, and the nature of the effect desired.

### Methods of Treatment

By another aspect, there is provided a method of treating PV or PF in a subject in need thereof, the method comprising administering to the subject a protein or polypeptide of the invention, thereby treating PV or PF in a subject.

By another aspect, there is provided a method of treating PV or PF in a subject in need thereof, the method comprising administering to the subject a protein complex of the invention, thereby treating PV or PF in a subject.

By another aspect, there is provided a method of treating PV or PF in a subject in need thereof, the method comprising administering to the subject a composition of the invention, thereby treating PV or PF in a subject.

In some embodiments, the administering is administering a pharmaceutical composition of the invention. In some embodiments, PV or PF is characterized by antibodies against the protein. In some embodiments, the protein is a target of PV or PF antibodies. It will be understood by the skilled artisan that a protein complex will be designed with fragments of proteins which are targeted by PV or PF antibodies in the subject. In some embodiments, antibodies are autoantibodies. In some embodiments, the disease is PV and the autoantibodies are against DSG1, DSG3 or both. In some embodiments, the disease is PF and the autoantibodies are against DSG1.

In some embodiments, treating comprises lowering antibody concentration. In some embodiments, treating comprises lower antibody number. In some embodiments, antibody concentration is circulating antibody concentration. In some embodiments, treating comprises depleting antibodies. In some embodiments, treating comprises killing B cells. In some embodiments, the B cell are autoreactive B cells. In some embodiments, killing B cells is specific B cell killing. In some embodiments, treating comprises killing B cells that produce the antibodies. In some embodiments, treating comprises killing B cells that produce the antibodies and the not substantially killing other B cells. In some embodiments, treating comprises killing B cell that produce antibodies against the protein complex. In some embodiments, treating comprises killing B cell that produce antibodies against the fragment. In some embodiments, treating comprises killing B cell that produce antibodies against a fragment of the protein complex.

In some embodiments, lowering antibodies comprises binding antibodies. In some embodiments, lowering is removing at least 10, 20, 25, 30, 40, 50, 60, 70, 75, 80, 90, 95, 97, 99 or 100% of the antibodies. Each possibility represents a separate embodiment of the invention. In some embodiments, antibodies are autoantibodies. In some embodiments, antibodies in antibodies in the subject. In some embodiments, antibodies are circulating antibodies. In some embodiments, autoantibodies are autoantibodies against the protein or fragment. In some embodiments, autoantibodies are cytotoxic autoantibodies. In some embodiments, autoantibodies comprise IgG1 autoantibodies. In some embodiments, autoantibodies comprise IgG3. In some embodiments, autoantibodies comprise IgG1 and IgG3 autoantibodies. In some embodiments, autoantibodies comprise IgG1, IgG2 and IgG3 autoantibodies. In some embodiments, autoantibodies comprise IgG1, IgG3 and IgG4 autoantibodies. In some embodiments, autoantibodies comprise IgG1, IgG2, IgG3 and IgG4 autoantibodies. In some embodiments, lowering is removing at least 25% of the antibodies. In some embodiments, lowering is removing at least 50% of the antibodies. In some embodiments, lowering is removing at least 70% of the antibodies. In some embodiments, lowering is removing at least 75% of the antibodies. In some embodiments, percent of the antibodies is percent of the autoantibodies. In some embodiments, percent of the antibodies is percent of the antibodies against the protein or fragment. In some embodiments, percent of the antibodies is percent of the antibodies associated with the disease.

In some embodiments, the method further comprises reducing antibodies in the subject. In some embodiments, the reducing is before the administering. In some embodiments, the reducing antibodies is reducing circulating antibodies. In some embodiments, the antibodies are autoantibodies. In some embodiments, the antibodies are against a protein. In some embodiments, the antibodies are against the protein that the fragment is from. In some embodiments, the antibodies are against the protein that at least one of the fragments is from. In some embodiments, the reducing is reducing antibodies against all proteins that at least one of the fragments are from. In some embodiments, the antibodies are against the protein complex. Methods of reducing antibodies are well known in the art and include, for example, plasmapheresis, intravenous Ig (IVIg), antibody filtering, and B cell targeting therapies, any of which may be employed. In some embodiments, the method comprises plasmapheresis of the antibodies before administering. In some embodiments, the method comprises administering a B cell targeting therapy before administering the therapeutic of the invention. In some embodiments, a B cell targeting therapy is an anti-B cell therapy. In some embodiments, the B cell targeting therapy is B cell lethal therapy. In some embodiments, the B cell targeting therapy is a pan B cell therapy. In some embodiments, the B cell targeting therapy is not a targeted therapy. As used herein, a “targeted B cell therapy” is a therapy that targets only specific B cell clones that produce specific antibodies. In some embodiments, an anti-B cell therapy is an anti-B cell antibody. B cell targeting antibodies are known in the art and include for non-limiting example, anti-CD20 antibodies. Anti-CD20 therapeutic antibodies are well known in the art and include, but are not limited to rituximab, ocrelizumab, obinutuzumab, ofatumumab, ibritumomab, tiuxetan, tositumomab, and ublituximab. In some embodiments, the B cell targeting therapy is rituximab.

### Nucleic Acids

By another aspect, there is provided a nucleic acid system comprising at least two nucleic acid molecules, wherein a first nucleic acid molecule encodes the first polypeptide chain of a protein complex of the invention and a second nucleic acid molecules encodes the second polypeptide chain of the protein complex of the invention.

By another aspect, there is provided a nucleic acid system comprising at least two nucleic acid molecules, wherein a first nucleic acid molecule encodes a first polypeptide chain comprising a fragment of a first human protein target of PV or PF autoantibodies or an analog or derivative thereof and a first dimerization domain and a second nucleic acid molecule encodes a second polypeptide chain comprising a fragment of a second human protein target of PV or PF autoantibodies or an analog or derivative thereof and second dimerization domain.

By another aspect, there is provided a nucleic acid molecule encoding a protein of the invention.

By another aspect, there is provided a nucleic acid molecule encoding a polypeptide chain of a composition of the invention.

By another aspect, there is provided a nucleic acid molecule encoding a composition of the invention.

By another aspect, there is provided a nucleic acid molecule encoding a fragment of a first protein target of PV or PF autoantibodies or an analog or derivative thereof and fragment of a second human protein target of PV or PF autoantibodies or an analog or derivative thereof.

In some embodiments, the nucleic acid system further comprises a third nucleic acid molecule that encodes a third polypeptide of the protein complex of the invention. In some embodiments, the nucleic acid system further comprises a fourth nucleic acid molecule that encodes a fourth polypeptide of the protein complex of the invention. In some embodiments, a first nucleic acid molecule encodes the first polypeptide of the invention. In some embodiments, a second nucleic acid molecule encodes the second polypeptide of the invention. In some embodiments, a third nucleic acid molecule encodes the third polypeptide of the invention. In some embodiments, a fourth nucleic acid molecule encodes the fourth polypeptide.

In some embodiments, the nucleic acid molecule is a vector. In some embodiments, the vector is an expression vector. In some embodiments, nucleic acid molecule comprises an open reading frame encoding the polypeptide chain. Expressing of an open reading frame within a cell is well known to one skilled in the art. It can be carried out by, among many methods, transfection, viral infection, or direct alteration of the cell's genome. Expression vectors are well known in the art and any vector compatible with a target cell in which the protein complex of the invention is being expressed may be used.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence. In some embodiments, the vector comprises a promoter. In some embodiments, the promoter is configured for expression in a target cell in which the protein complex of the invention is being expressed.

The vector may be a DNA plasmid delivered via non-viral methods or via viral methods. The viral vector may be a retroviral vector, a herpesviral vector, an adenoviral vector, an adeno-associated viral vector or a poxviral vector. The promoter may be active in mammalian cells. The promoters may be a viral promoter. The promoter may be active in bacterial cells. The promoter may be active in human cells. The promoter may be active in fibroblasts. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, the open reading frame is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2 (±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, the nucleic acid molecule is a single nucleic acid molecule. In some embodiments, the first and second nucleic acid molecules are different molecules. In some embodiments, the first and second nucleic acid molecule are the same molecule. In some embodiments, any two of the first, second, third and fourth nucleic acid molecules are different molecules. In some embodiments, any two of the first, second, third and fourth nucleic acid molecules are the same molecule. In some embodiments, any three of the first, second, third and fourth nucleic acid molecules are different molecules. In some embodiments, the first, second, and third nucleic acid molecules are different molecules. In some embodiments, any three of the first, second, third and fourth nucleic acid molecules are the same molecule. In some embodiments, all of the first, second, third and fourth nucleic acid molecules are different molecules. In some embodiments, all of the first, second, third and fourth nucleic acid molecules are the same molecule.

### Methods of Production

By another aspect, there is provided a method for producing a protein, the method comprising:
obtaining a first fragment of an extracellular domain of a first human receptor or an analog or derivative thereof, and a second fragment of an extracellular domain of a second human receptor or analog or derivative thereof, wherein the first and second human receptors are targets of PV or PF autoantibodies and linking the first fragment to the second fragment to produce a single polypeptide chain;
   thereby producing a protein

By another aspect, there is provided a method for producing a protein, the method comprising: obtaining a first fragment of an extracellular domain of a first human protein target of Pemphigus vulgaris/foliaceus autoantibodies or an analog or derivative thereof and linking the first fragment to an effector moiety that is not an unmodified Fc domain.

By another aspect, there is provided a method for producing a protein, the method comprising:
obtaining a first fragment of an extracellular domain of DSG1 or DSG3 or an analog or derivative thereof, and truncating the DSG1 or DSG3 or an analog or derivative thereof wherein the truncation decreases aggregation of the first fragment;
   thereby producing a protein.

By another aspect, there is provided a method for producing a protein, the method comprising:
obtaining a first fragment of an extracellular domain of DSG1 or DSG3 or an analog or derivative thereof, and truncating the DSG1 or DSG3 or an analog or derivative thereof wherein the truncation decreases aggregation of the first fragment and linking the truncated fragment to an effector moiety that is not an unmodified Fc domain;
   thereby producing a protein.

By another aspect, there is provided a method for producing a protein, the method comprising:
obtaining a first fragment of an extracellular domain of a first human receptor or an analog or derivative thereof, and a second fragment of an extracellular domain of a second human receptor or analog or derivative thereof, wherein the first and second human receptors are targets of Pemphigus vulgaris/foliaceus autoantibodies and different proteins and linking the first fragment to the second fragment to produce a single polypeptide chain and further linking the polypeptide chain to an effector moiety that is not an unmodified Fc domain;
   thereby producing a protein.

By another aspect, there is provided a method for producing a protein, the method comprising:
obtaining a first fragment of an extracellular domain of DSG1 or DSG3 or an analog or derivative thereof, and producing a mutation in the DSG1 or DSG3 or an analog or derivative thereof wherein the mutation decreases aggregation of the first fragment;
   thereby producing a protein.

By another aspect, there is provided a method for producing a protein, the method comprising:
obtaining a first fragment of an extracellular domain of DSG1 or DSG3 or an analog or derivative thereof and generating in the first fragment at least one mutation that decreases aggregation of the first fragment to produce a mutant fragment and linking the mutant fragment to an effector moiety that is not an unmodified Fc domain;
   thereby producing a protein.

By another aspect, there is provided a method for producing a protein, the method comprising:
a. obtaining a fragment of an extracellular domain of DSG1 or an analog or derivative thereof or a fragment of an extracellular domain of DSG3 or an analog or derivative thereof;
b. generating at least one mutation in the fragment to produce a mutated fragment;
c. measuring solubility, aggregation or both of the mutated fragment; and
d. selecting at least one mutated fragment with increased solubility, decreased aggregation or both as compared to the obtained fragment;
thereby producing a protein.

By another aspect, there is provided a method for producing a protein, the method comprising:
a. obtaining a fragment of an extracellular domain of DSG1 or an analog or derivative thereof or a fragment of an extracellular domain of DSG3 or an analog or derivative thereof;
b. generating at least one mutation in the fragment to produce a mutated fragment;
c. measuring solubility, aggregation or both of the mutated fragment; and
d. selecting at least one mutated fragment with increased solubility, decreased aggregation or both as compared to the obtained fragment;
e. and linking the selected mutated fragment to an effector moiety that is not an unmodified Fc domain;
thereby producing a protein.

By another aspect, there is provide a method for producing a protein complex, the method comprising:
obtaining a first fragment of a first protein target of PV or PF autoantibodies or an analog or derivative thereof and a second fragment of a second protein target of PV or PF autoantibodies or an analog or derivative thereof, linking the first fragment to a first dimerization domain to produce a first polypeptide and linking the second fragment to a second dimerization domain to produce a second polypeptide chain;
   thereby producing a protein complex.

By another aspect, there is provide a method for producing a protein complex, the method comprising:
obtaining a first fragment of a first protein target of PV or PF autoantibodies or an analog or derivative thereof and a second fragment of a second protein target of PV or PF autoantibodies or an analog or derivative thereof, linking the first fragment to a first dimerization domain to produce a first polypeptide and linking the second fragment to a second dimerization domain to produce a second polypeptide chain and linking either the first polypeptide chain, the second polypeptide chain or both to an effector moiety that is not an unmodified Fc domain;
   thereby producing a protein complex.

By another aspect, there is provided a method for producing a protein, the method comprising:
culturing a host cell comprising one or more vectors comprising a nucleic acid sequence encoding a single polypeptide chain, wherein the single polypeptide chain is produced by:
   i. obtaining a first fragment of an extracellular domain of a first human receptor or an analog or derivative thereof and a second fragment of an extracellular domain of a second human receptor or analog or derivative thereof, wherein the first and second human receptors are targets of PV or PF autoantibodies and are different proteins; and
   ii. linking the first fragment to the second fragment to produce a single polypeptide chain;
      thereby producing a protein.

By another aspect, there is provided a method for producing a protein, the method comprising:
culturing a host cell comprising one or more vectors comprising a nucleic acid sequence encoding a single polypeptide chain, wherein the single polypeptide chain is produced by:
   i. obtaining a first fragment of an extracellular domain of a first human receptor or an analog or derivative thereof and a second fragment of an extracellular domain of a second human receptor or analog or derivative thereof, wherein the first and second human receptors are targets of PV or PF autoantibodies and are different proteins;
   ii. linking the first fragment to the second fragment to produce a single polypeptide chain; and
   iii. linking the single polypeptide chain to an effector moiety that is not an unmodified Fc domain;
      thereby producing a protein.

By another aspect, there is provide a method for producing a protein complex, the method comprising:
culturing a host cell comprising one or more vectors comprising a nucleic acid sequence encoding at least two polypeptide chains, wherein the two polypeptide chains are produced by:
   i. obtaining a first fragment of a first protein target of PV or PF autoantibodies or an analog or derivative thereof and a second fragment of second protein target of PV or PF autoantibodies or analog or derivative thereof; and
   ii. linking the first fragment to a first dimerization domain to produce a first polypeptide chain and linking the second fragment to a second dimerization domain to produce a second polypeptide chain;
      thereby producing a protein complex.

By another aspect, there is provide a method for producing a protein complex, the method comprising:
culturing a host cell comprising one or more vectors comprising a nucleic acid sequence encoding at least two polypeptide chains, wherein the two polypeptide chains are produced by:
   i. obtaining a first fragment of a first protein target of PV or PF autoantibodies or an analog or derivative thereof and a second fragment of second protein target of PV or PF autoantibodies or analog or derivative thereof;
   ii. linking the first fragment to a first dimerization domain to produce a first polypeptide chain and linking the second fragment to a second dimerization domain to produce a second polypeptide chain; and
   iii. linking the first polypeptide chain, the second polypeptide chain or both to an effector moiety that is not an Fc domain.
      thereby producing a protein complex.

In some embodiments, the protein is a protein of the invention. In some embodiments, the protein is a polypeptide. In some embodiments, the truncation is a truncation of the invention. In some embodiments, the protein is a polypeptide of the invention. In some embodiments, the mutation is a mutation of the invention. In some embodiments, the protein complex is a protein complex of the invention. In some embodiments, the protein composition is a composition of the invention. In some embodiments, the protein is a protein of the invention. In some embodiments, the protein is a polypeptide chain of the invention. In some embodiments, the fragment is a fragment of the invention. In some embodiments, the derivative is a derivative of the invention. In some embodiments, the analog is an analog of the invention. In some embodiments, the dimerization domain is a dimerization domain of the invention. In some embodiments, the composition, protein complex, protein, fragment, analog, derivative or dimerization domain is such as is described hereinabove. In some embodiments, the method further comprises linking the protein, polypeptide or protein complex to an effector moiety. In some embodiments, the effector moiety is not an Fc domain. In some embodiments, the effector moiety does not comprise an Fc moiety. In some embodiments, the effector moiety is not an unmodified Fc domain. In some embodiments, the effector moiety is an Fc domain comprising at least one mutation that increases ADCC.

In some embodiments, the protein is a human protein. In some embodiments, the protein is a cell surface protein. In some embodiments, the first and second protein are the same protein. In some embodiments, the first and second protein are different proteins. In some embodiments, the first and second proteins are targets of PV or PF autoantibodies. In some embodiments, the first and second proteins are targets of autoantibodies associated with PV or PF. In some embodiments, PV or PF is characterized by autoantibodies against the first and second proteins. In some embodiments, the protein is a receptor, and the fragment is a fragment of the extracellular domain. In some embodiments, the fragment comprises a fragment of the extracellular domain. In some embodiments, the fragment consists of the extracellular domain.

In some embodiments, the first and second dimerization domains are capable of dimerizing to each other. In some embodiments, the first and second dimerization domains are configured to dimerize with each other. In some embodiments, the method further comprises contacting the first and second polypeptides. In some embodiments, the contacting comprises incubating the polypeptides together. In some embodiments, the contacting is in a cell. In some embodiments, the contacting is in vitro. In some embodiments, the contacting is under conditions sufficient to allow dimerization. In some embodiments, allowing is inducing. In some embodiments, the conditions are sufficient to allow dimerization of the polypeptides. In some embodiments, the conditions are physiological conditions.

In some embodiments, the method further comprises inserting a third dimerization domain into the first polypeptide. In some embodiments, inserting is linking. In some embodiments, inserting is inserting a nucleic acid sequence encoding the third dimerization domain into a nucleic acid molecule or vector encoding the first polypeptide. In some embodiments, the linking is linking the third dimerization domain to the first dimerization domain. In some embodiments, the linking is linking the third dimerization domain to the first fragment.

In some embodiments, the method further comprises obtaining a third fragment of a third protein target of PV or PF autoantibodies or an analog or derivative thereof and linking it to a fourth dimerization domain to produce a third polypeptide chain. In some embodiments, the third and fourth dimerization domains are capable of dimerization to each other. In some embodiments, the third and fourth dimerization domains are configured to dimerize to each other. In some embodiments, the method further comprises contacting the first, second and third polypeptide chains. In some embodiments, the method further comprises expressing in the host cell a nucleic acid sequence encoding a third polypeptide chain. In some embodiments, the third polypeptide chain is produced by obtaining a third fragment of a third protein and linking it to a fourth dimerization domain to produce a third polypeptide chain. In some embodiments, the method comprises expression the first, second and third polypeptide chains in a cell.

In some embodiments, the method further comprises inserting a fifth dimerization domain into the second polypeptide. In some embodiments, inserting is linking. In some embodiments, inserting is inserting a nucleic acid sequence encoding the fifth dimerization domain into a nucleic acid molecule or vector encoding the second polypeptide. In some embodiments, the linking is linking the fifth dimerization domain to the second dimerization domain. In some embodiments, the linking is linking the fifth dimerization domain to the second fragment.

In some embodiments, the method further comprises obtaining a fourth fragment of a fourth protein target of PV or PF autoantibodies or an analog or derivative thereof and linking it to a sixth dimerization domain to produce a fourth polypeptide chain. In some embodiments, the fifth and sixth dimerization domains are capable of dimerization to each other. In some embodiments, the fifth and sixth dimerization domains are configured to dimerize to each other. In some embodiments, the method further comprises contacting the first, second, third and fourth polypeptide chains. In some embodiments, the method further comprises expressing in the host cell a nucleic acid sequence encoding a fourth polypeptide chain. In some embodiments, the fourth polypeptide chain is produced by obtaining a fourth fragment of a fourth protein and linking it to a sixth dimerization domain to produce a fourth polypeptide chain. In some embodiments, the method comprises expression the first, second, third and fourth polypeptide chains in a cell.

In some embodiments, the method further comprises inserting an Fc region into the first chain. In some embodiments, the method further comprises inserting an Fc region into the second chain. In some embodiments, the method further comprises inserting an Fc region into the third chain. In some embodiments, the method further comprises inserting an Fc region into the fourth chain. In some embodiments, the method further comprises inserting a portion of an Fc region into the first chain and a portion of the Fc region into the second chain wherein and interface of the two portions produces a complete Fc region. In some embodiments, the Fc region is not an unmodified Fc region. In some embodiments, the Fc region comprises at least one mutation that increases ADCC.

In some embodiments, an Fc region is inserted C-terminally to a dimerization domain. In some embodiments, an Fc region is inserted C-terminally to a fragment. In some embodiments, an Fc region is inserted N-terminally to a dimerization domain. In some embodiments, an Fc region is inserted N-terminally to a fragment. In some embodiments, a fragment is inserted or linked C-terminally to a dimerization domain. In some embodiments, a fragment is inserted or linked N-terminally to a dimerization domain.

In some embodiments, the method further comprises inserting a linker between at least two sections of a polypeptide chain. In some embodiments, the linker is inserted between a fragment and a dimerization domain. In some embodiments, the linker is inserted between a fragment and an Fc region. In some embodiments, the linker is inserted between an Fc region and a dimerization domain. In some embodiments, the linker is inserted between a dimerization domain and another dimerization domain. In some embodiments, the linker is inserted between a fragment and another fragment. In some embodiments, the linker is inserted between a fragment of a first protein and a fragment of a second protein.

In some embodiments, the method further comprises producing at least one mutation in the protein. In some embodiments, the mutation is made in fragment. In some embodiments, the mutation is made in extracellular domain. In some embodiments, the mutation is made in a cadherin domain of the fragment. In some embodiments, the method further comprises truncating the protein. In some embodiments, the method further comprises truncating the fragment. In some embodiments, the method further comprises truncating the extracellular domain. In some embodiments, the truncation removes at least one extracellular functional domain. In some embodiments, the truncation removes at least one cadherin domain.

In some embodiments, the method further comprises measuring aggregation of the protein. In some embodiments, the method further comprises measuring aggregation of the mutated protein. In some embodiments, the method further comprises measuring aggregation of the truncated protein. In some embodiments, the method further comprises selecting a protein with low aggregation. In some embodiments, low aggregation is aggregation below a predetermined threshold. In some embodiments, low aggregation is non-substantial aggregation. In some embodiments, the method further comprises selecting a mutated protein with reduced aggregation. In some embodiments, the method further comprises selecting a truncated protein with reduced aggregation. In some embodiments, reduced is as compared to the non-mutated or non-truncated protein. In some embodiments, reduced is as compared to a control protein. In some embodiments, reduced is as compared to a WT extracellular domain. In some embodiments, reduced comprises at least a 5, 10, 15, 20, 25, 30, 35, 40, 45, 50, 55, 60, 65, 70, 75, 80, 85, 90, 92, 95, 97, 99 or 100% reduction. Each possibility represents a separate embodiment of the invention. In some embodiments, reduced comprises at least a 50% reduction. In some embodiments, reduced comprises at least a 25% reduction. Methods of measuring aggregation are well known in the art and any such method may be employed, including those provided herein. In some embodiments, aggregation is measured in non-reducing conditions.

In some embodiments, the method further comprises measuring solubility, aggregation, or both of the obtained fragment. In some embodiments, increased is significantly increased. In some embodiments, decreased is reduced. In some embodiments, decreased is significantly decreased. In some embodiments, significantly is statistically significantly.

In some embodiments, the protein is for use in a method of the invention. In some embodiments, the polypeptide is for use in a method of the invention. In some embodiments, the protein complex is for use in a method of the invention. In some embodiments, the method is a therapeutic method. In some embodiments, the method is a diagnostic method. In some embodiments, the method is a method of treatment. In some embodiments, the method is a method of determining suitability for treatment.

By another aspect, there is provided a protein complex produced by a method of the invention.

By another aspect, there is provided a protein produced by a method of the invention.

By another aspect, there is provided a composition produced by a method of the invention.

Patient selection

By another aspect, there is provided a method of determining suitability of a subject to be treated by a method of the invention, the method comprising receiving a sample from the subject, contacting the sample with a composition of the invention and determining binding of antibodies within the sample to the composition, wherein binding of the antibodies to the composition indicates the subject is suitable to be treated by a method of the invention, thereby determining suitability of the subject to be treated.

By another aspect, there is provided a method of determining suitability of a subject to be treated by a method of the invention, the method comprising receiving a sample from the subject, contacting the sample with a protein complex of the invention and determining binding of antibodies within the sample to the protein complex, wherein binding of the antibodies to the protein complex indicates the subject is suitable to be treated by a method of the invention, thereby determining suitability of the subject to be treated.

By another aspect, there is provided a method of determining suitability of a subject to be treated by a method of the invention, the method comprising receiving a sample from the subject, contacting the sample with a protein or polypeptide of the invention and determining binding of antibodies within the sample to the protein, wherein binding of the antibodies to the protein or polypeptide indicates the subject is suitable to be treated by a method of the invention, thereby determining suitability of the subject to be treated.

In some embodiments, the subject is a subject in need thereof. In some embodiments, the subject is a subject such as described hereinabove. In some embodiments, the subject suffers from PV or PF. In some embodiments, the subject is known to be positive for autoantibodies associated with PV or PF. In some embodiments, the subject is seropositive. In some embodiments, the subject is seronegative. In some embodiments, the subject is naïve to treatment. In some embodiments, the treatment is treatment for PV or PF. In some embodiments, the subject has received treatment and has relapsed.

In some embodiments, the method comprises obtaining the sample from the subject. In some embodiments, the sample comprises tissue. In some embodiments, the sample is a biopsy. In some embodiments, the sample is a bodily fluid. In some embodiments, the bodily fluid is blood. In some embodiments, the bodily fluid is serum. In some embodiments, the bodily fluid is plasma. In some embodiments, the bodily fluid is a fluid that comprises antibodies. In some embodiments, the bodily fluid is selected from at least one of: blood, serum, plasma, intestinal fluid, saliva, tumor fluid, urine, interstitial fluid, cerebral spinal fluid and stool.

In some embodiments, contacting is incubating. In some embodiments, contacting is under conditions sufficient for binding of antibodies to the protein complex. In some embodiments, conditions comprise a time sufficient for binding of antibodies to the protein complex. In some embodiments, conditions comprise physiological conditions. In some embodiments, the protein complex is added to the sample. In some embodiments, the protein complex is dissolved in the bodily fluid. In some embodiments, the antibodies are autoantibodies. In some embodiments, the antibodies are antibodies against a protein.

In some embodiments, the composition further comprises a detectable moiety. In some embodiments, the protein complex further comprises a detectable moiety. In some embodiments, the protein further comprises a detectable moiety. In some embodiments, the method further comprises contacting the composition, complex and/or protein with a peptide comprising a detectable moiety. In some embodiments, the peptide is configured to bind the composition, protein and/or complex. In some embodiments, the peptide is specific to the composition, protein and/or complex. As used herein, the term “specific binding” refers to binding to a specific molecule to the exclusion of other molecules. In some embodiments, the peptide is specific to the composition, protein and/or complex to the exclusion of other proteins in the sample. In some embodiments, the peptide is specific to the composition, protein and/or complex to the exclusion of naturally occurring antibodies in the sample. In some embodiments, the peptide is specific to the composition, protein and/or complex to the exclusion of the antibodies in the sample. In some embodiments, the determining binding comprises detecting the moiety. In some embodiments, the determining comprises isolating the protein complex. In some embodiments, the determining comprises eluting antibodies from the complex. Methods of protein identification are well known in the art and any such method may be used. Examples of such method include western blotting, ELISA, FACS analysis and protein sequencing, such as by mass spectrometry. In some embodiments, the determining comprises ELISA. In some embodiments, the ELISA is a competitive ELISA. In some embodiments, the competitive ELISA comprises competition with antibodies. In some embodiments, the antibodies are antibodies associated with the disease.

In some embodiments, binding is positive binding. In some embodiments, binding is binding above a predetermined threshold. In some embodiments, binding is specific binding. In some embodiments, binding is binding to at least one of the fragments of the protein complex. In some embodiments, binding is binding to at least two of the fragments of the protein complex. In some embodiments, binding is binding to at least three of the fragments of the protein complex. In some embodiments, binding is binding to at least four of the fragments of the protein complex. In some embodiments, binding of at least 10, 20, 25, 30, 40, 50, 60, 70, 75, 80, 90, 95, 97, 99 or 100% of the antibodies in the sample. Each possibility represents a separate embodiment of the invention. In some embodiments, binding of at least 50% of the antibodies in the sample. In some embodiments, binding of at least 70% of the antibodies in the sample. In some embodiments, binding of at least 75% of the antibodies in the sample. In some embodiments, percent of the antibodies is percent of the autoantibodies. In some embodiments, percent of the antibodies is percent of the antibodies against the protein. In some embodiments, percent of the antibodies is percent of the antibodies associated with the disease.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Itis appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, immunological, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Example 1

Autoantibodies to DSG1 and DSG3 are the primary cause of PV, while DSG1 autoantibodies are the primary cause of PF. Although it had previously been thought that DSG3 autoantibodies were the main driver of PV as well, it has become increasingly clear in recent years that both proteins play an important role. Indeed, many patients have autoantibodies against both proteins. Desmogleins are involved in tight junction formation and thus their extracellular domains contain several interacting domains (cadherin-like domains) that allow for homo-adhesion (adhesion between one type of Desmoglein, e.g., DSG1/DSG1 adhesion or DSG3/DSG3 adhesion) as well as hetero-adhesion between different Desmogleins. DSG1 and DSG3 each contain an N-terminal pro-domain that must be cleaved for normal function. After this, both proteins contain 4 separate binding domains in their extracellular region. Therapeutic agents were therefore developed that target DSG1 alone, DSG3 alone and both proteins at the same time. Various combinations of the extracellular domains were tested, along with various mutations to reduce homo- and hetero-adhesion.

Long term remission for PV patients would need to remove a significant proportion of autoreactive B cells which produce the autoantibody pool. Simply removing the autoantibodies from circulation, while potentially effective in treating the symptoms of PV/PF, would require repeated treatments for the rest of the subject's life as the long-lived B cells would perpetually continue to make new autoantibodies. Importantly, the B cells that produced the autoantibodies express a B-cell receptor (BCR) on their surfaces which is an identical membrane-bound form of these autoantibodies. This allows the B cells themselves to be targeted by a therapeutic that contains the autoantibody BCR-specific epitope(s). By coupling the target epitope to the Fc region of the antibody heavy chain, a therapeutic can direct specific killing of autoantibody producing B cells. This approach is also robust to potential evasion of specific subpopulations, which occurs when using agents that are targeting specific differentiation markers on the cell surface (e.g., CD19, CD38, BCMA), as every cell carrying the autoreactive BCR will be targeted regardless of its differentiation state. This approach is also beneficial in protecting and preserving non-autoreactive, including protective (e.g., anti-viral, anti-bacterial) subpopulations, which are damaged by treatments that are targeting nonspecific differentiation markers (e.g., CD20, CD38, BCMA) regardless of whether or not they are carrying an autoreactive BCR.

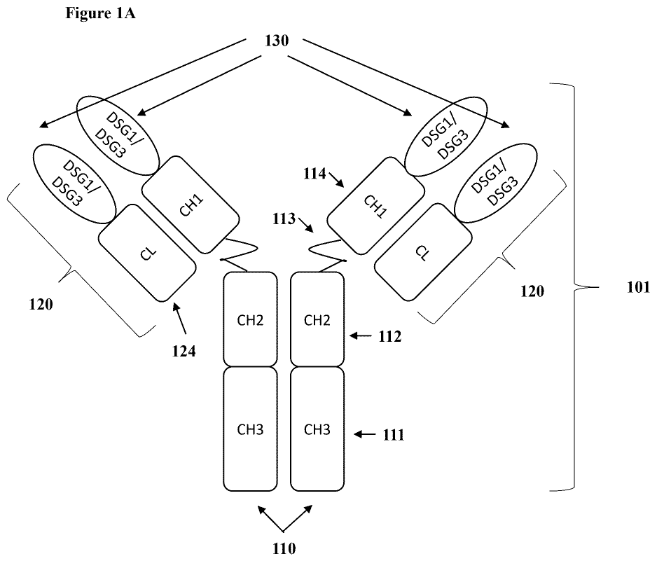

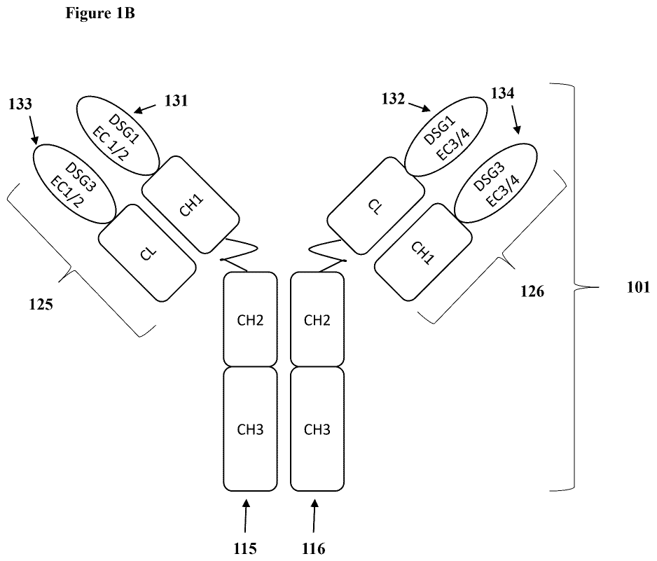

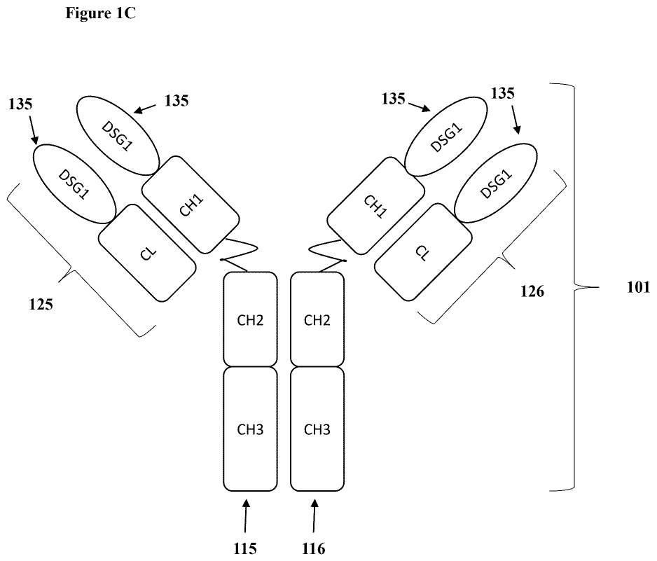

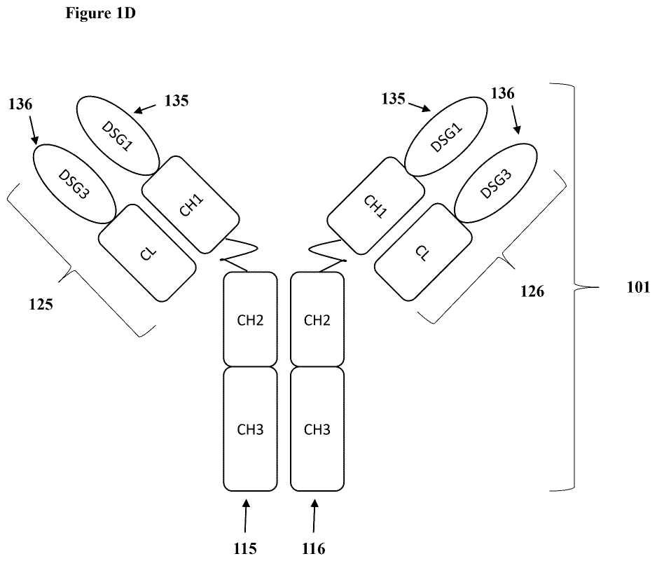

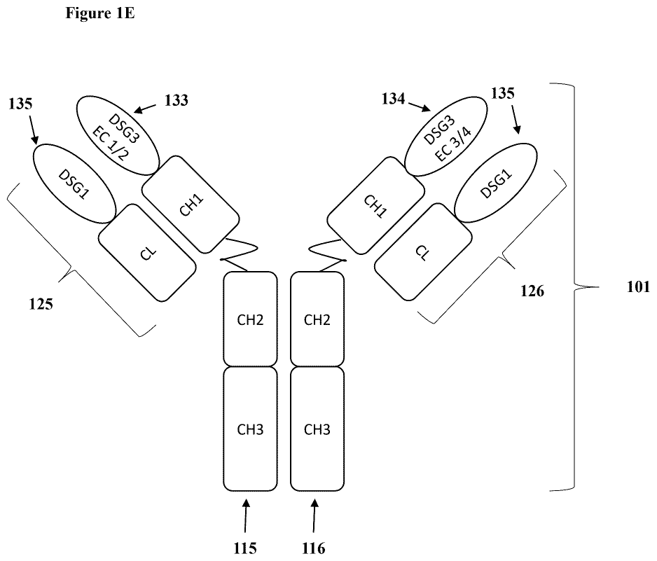

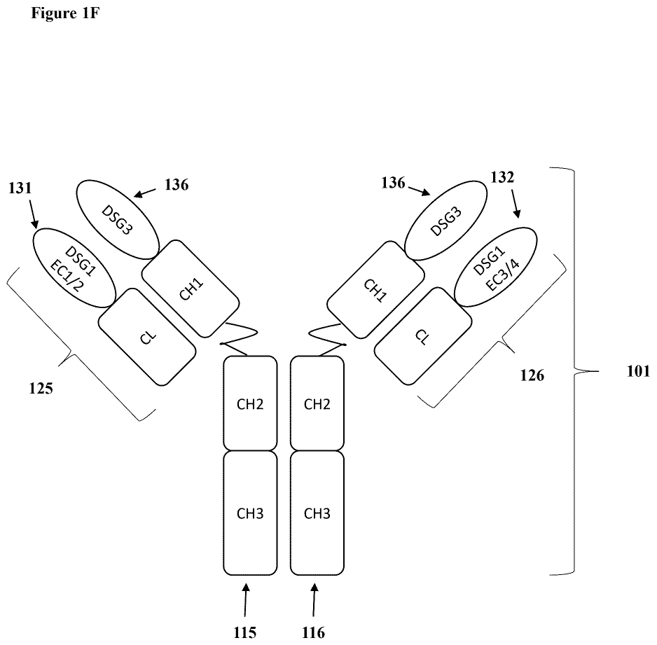

FIG. 1A shows one embodiment of the therapeutic agent of the invention. Immunoglobulin (Ig)-like protein complex 101 comprises four polypeptide chains: two heavy-chain-like polypeptides 110 and two light-chain-like polypeptides 120. Chains 110 are able to dimerize via disulfide bonds between them. Further, chains 110 may comprise any or all of CH3 domain 111, CH2 domain 112, hinge region 113 and CH1 domain 114. In this embodiment, the CH3 domain 111, CH2 domain 112, and hinge region 113 all comprise disulfide bonds and act as dimerization domains, though use of other dimerization domains is also possible. These domains are well known in the art and can be selected from any of human (or non-human) IgG1, IgG2, IgG3, IgG4, IgA, IgM, IgE, and IgD domains for example. A skilled artisan will appreciate that the Fc portion of IgG1 and IgG3 incorporated into chain 110 will allow the molecule to induce antibody dependent cellular cytotoxicity (ADCC) and complement dependent cytotoxicity (CDC). Chains 120 are able to dimerize with chains 110 via disulfide bonds found in CH1 domain 114 and CL domain 124.

Chains 110 and 120 are devoid of variable regions, unlike naturally occurring or manmade antibodies. In place of the variable region each chain has a fragment 130 from the extracellular portion of the human Desmoglein 1 or Desmoglein 3. Each chain can be generated to have the same fragment or derivative of DSG1 or DSG3 or different fragments/derivatives. Indeed, as shown in FIG. 1B, the two heavy chains 115 and 116 can be engineered separately such that chain 115 contains, for example extracellular domains (EC) 1 and 2 of DSG1 131 and chain 116 contains EC3 and 4 of DSG1 132. The same is true for light chains 125 and 126, which, for example, can contain the DSG3 EC1 and 2 133 and EC2 and EC4 134, respectively. Thus, the therapeutic molecule can be designed with four copies of a protein or domain (FIG. 1C), two copies each of two different proteins or domains (FIG. 1D), or one copy each of four different protein fragments or domains (FIG. 1B) or any other combination thereof. Indeed, the molecule is sufficiently modular that it could be engineered with three copies of one protein/domain and one copy of another, or two copies of one protein/domain and one copy of two others. FIG. 1E shows embodiments where the two light chains are identical, but the two heavy chains are different. And FIG. 1F shows embodiments where the two heavy chains are identical, and the two light chains are different. Importantly, the therapeutic molecule can be engineered to comprise any combination of the various domains of the two proteins which will be able to bind autoantibodies from a wide variety of patients and not just some patients. It will be understood by a skilled artisan that any chain can include any protein, fragment, domain or variant, and the combinations of chains and subunit depicted in FIGS. 1A-1F are meant only to be illustrative and not limiting.

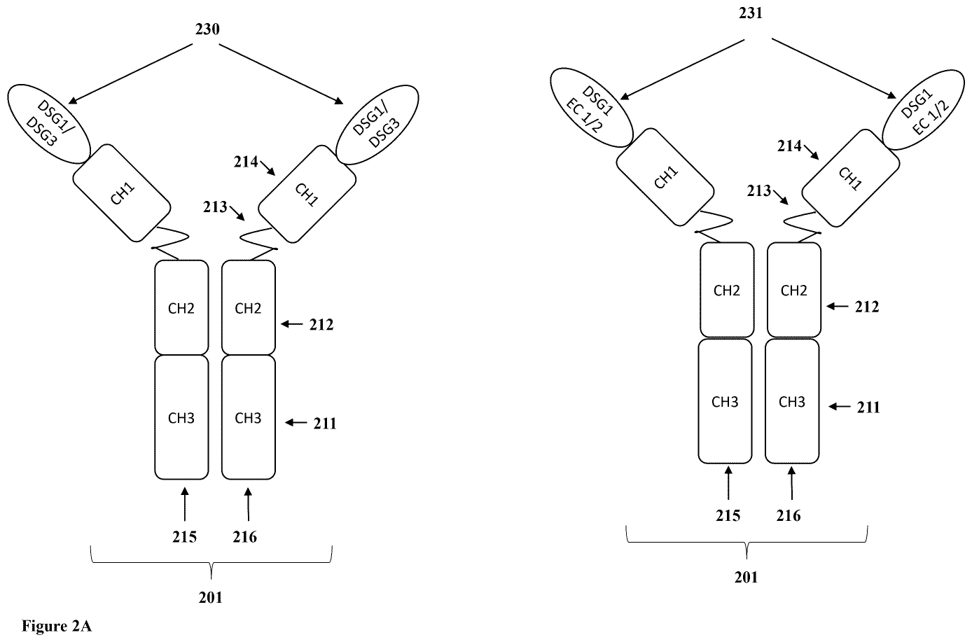

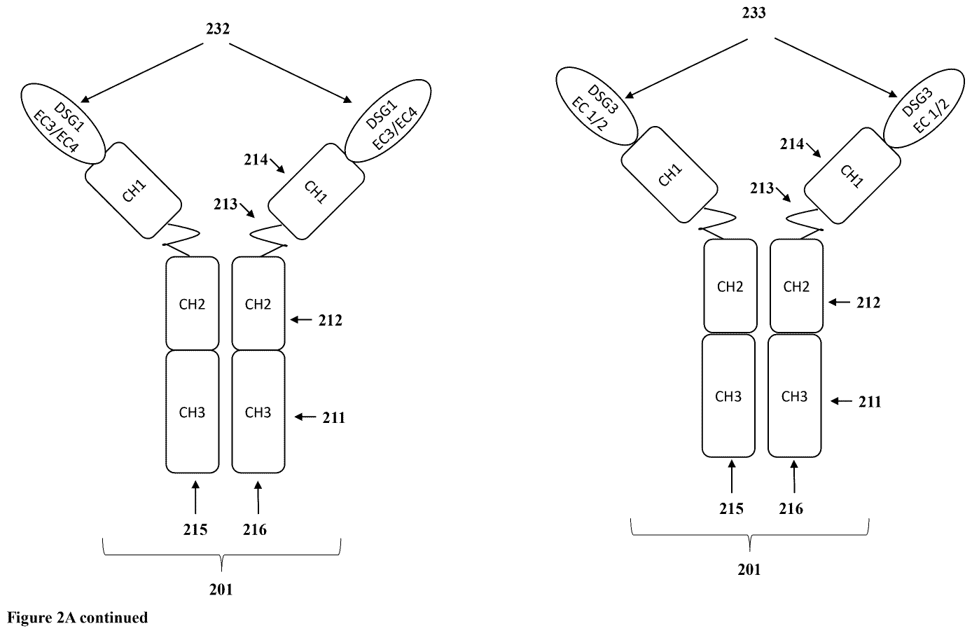

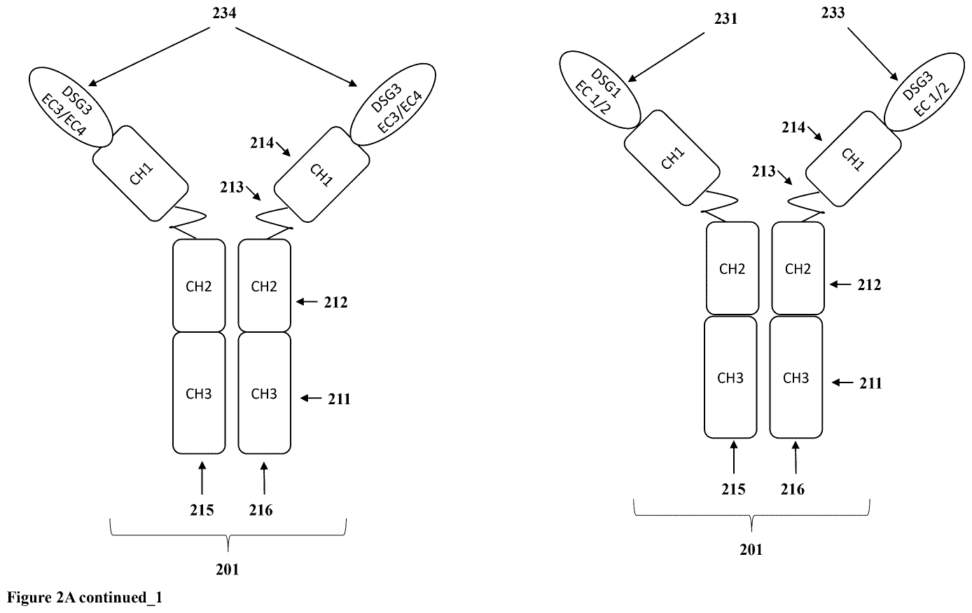

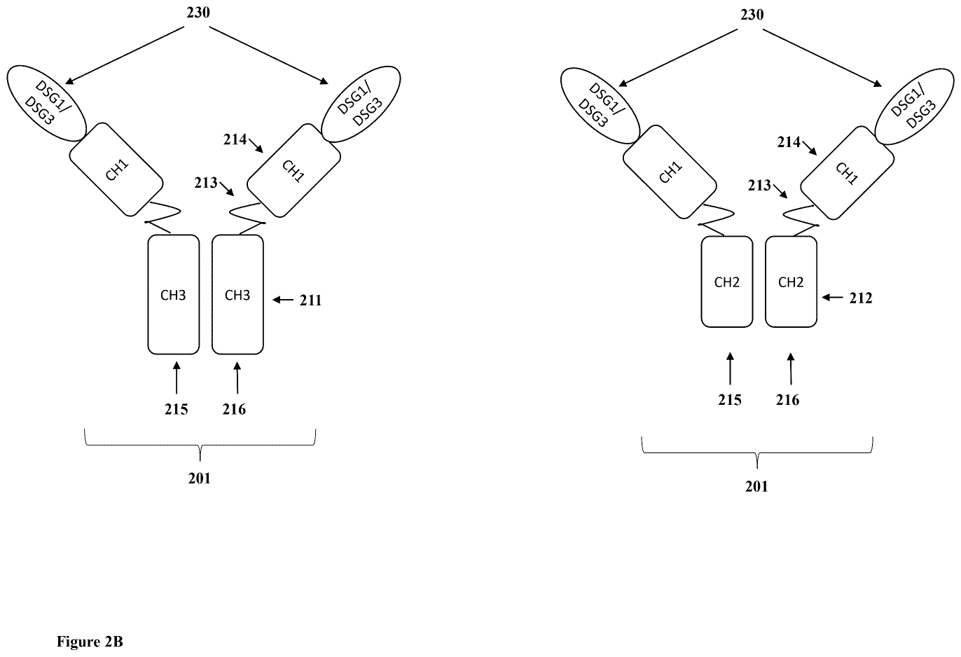

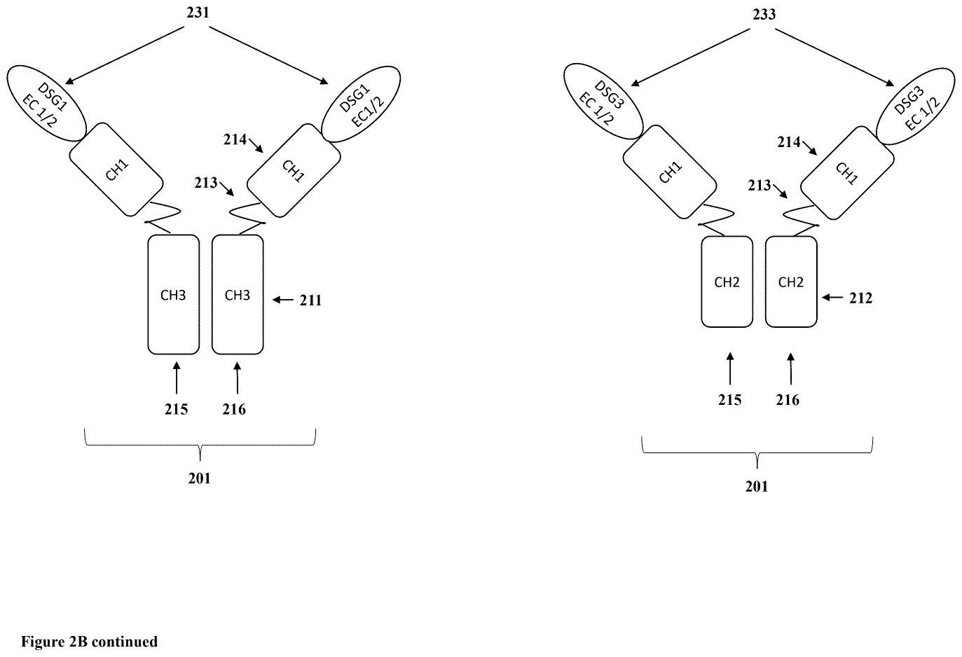

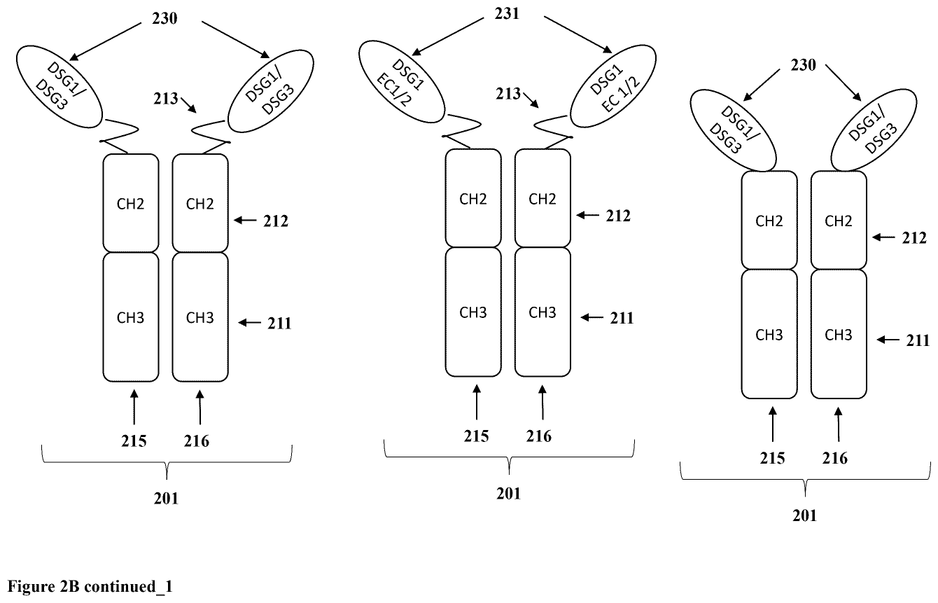

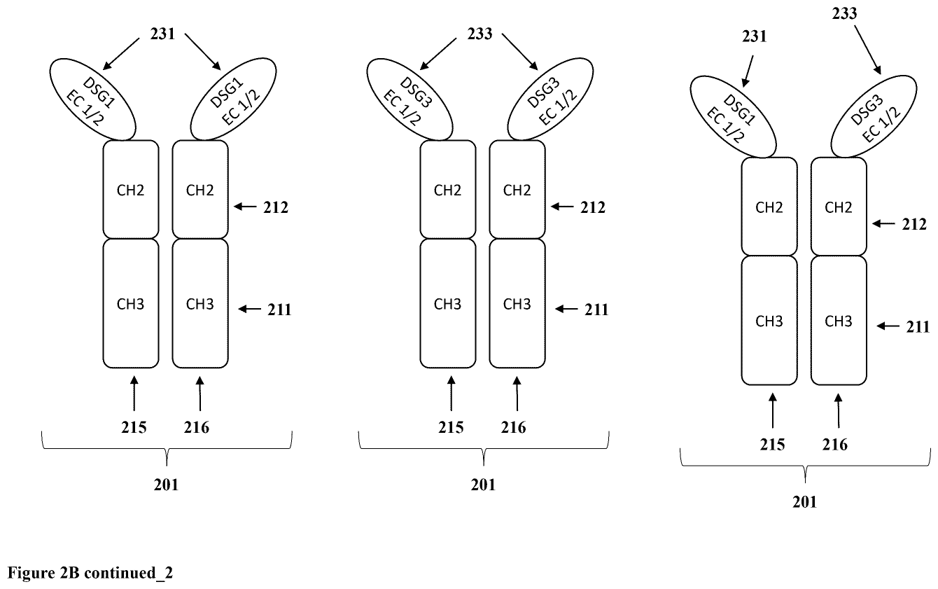

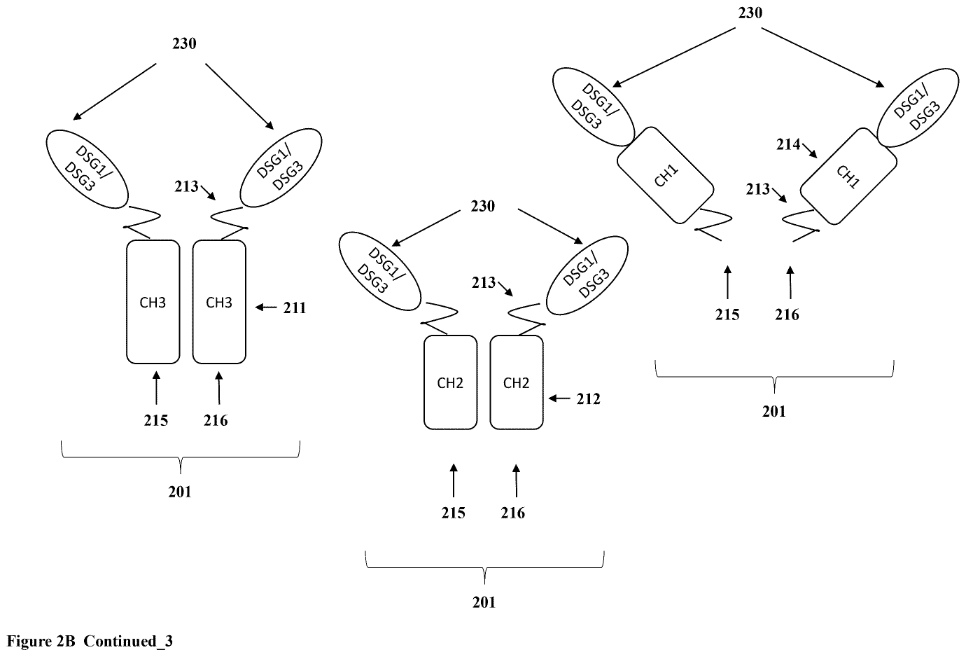

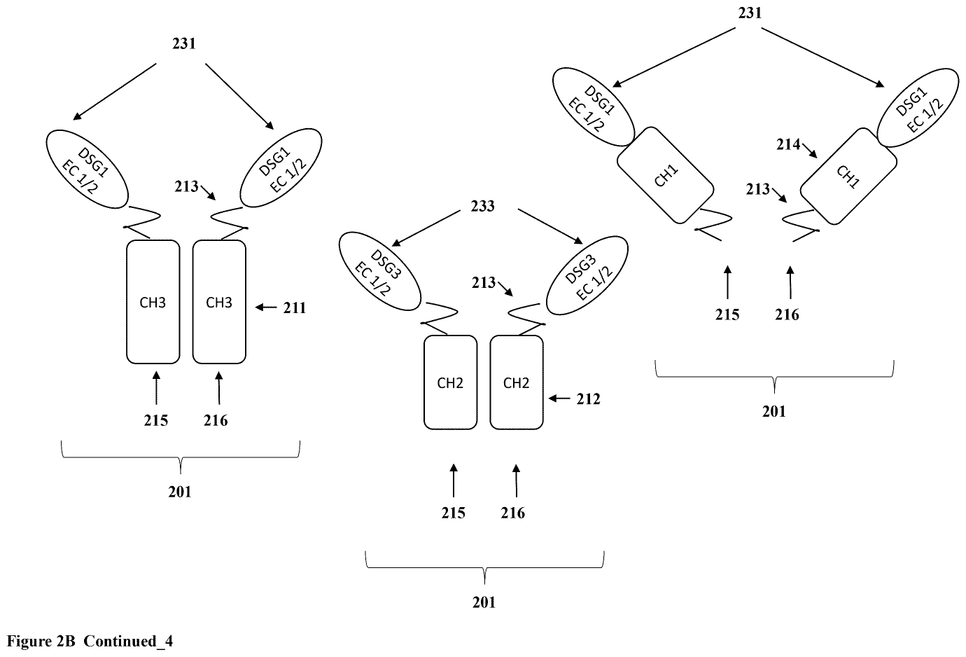

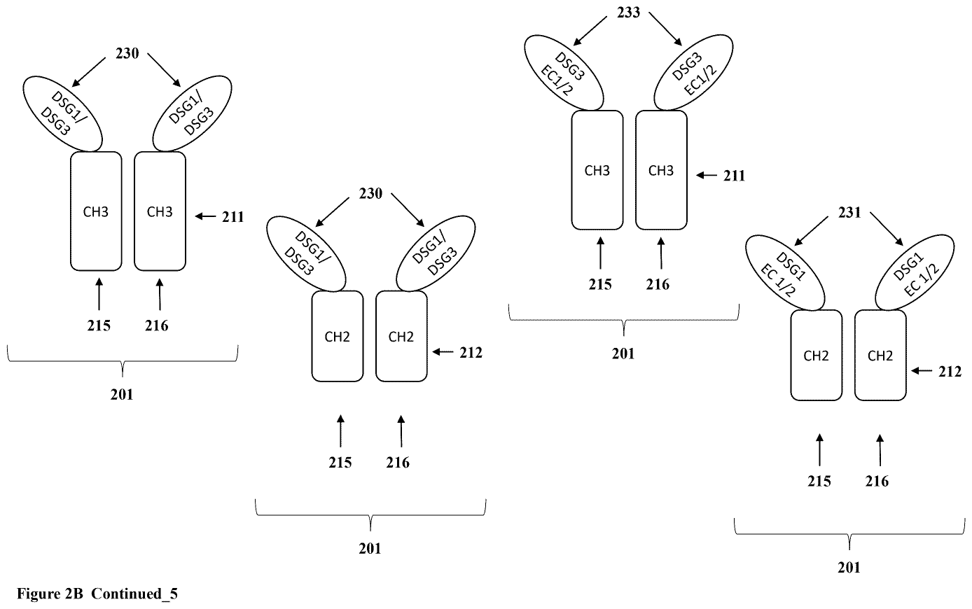

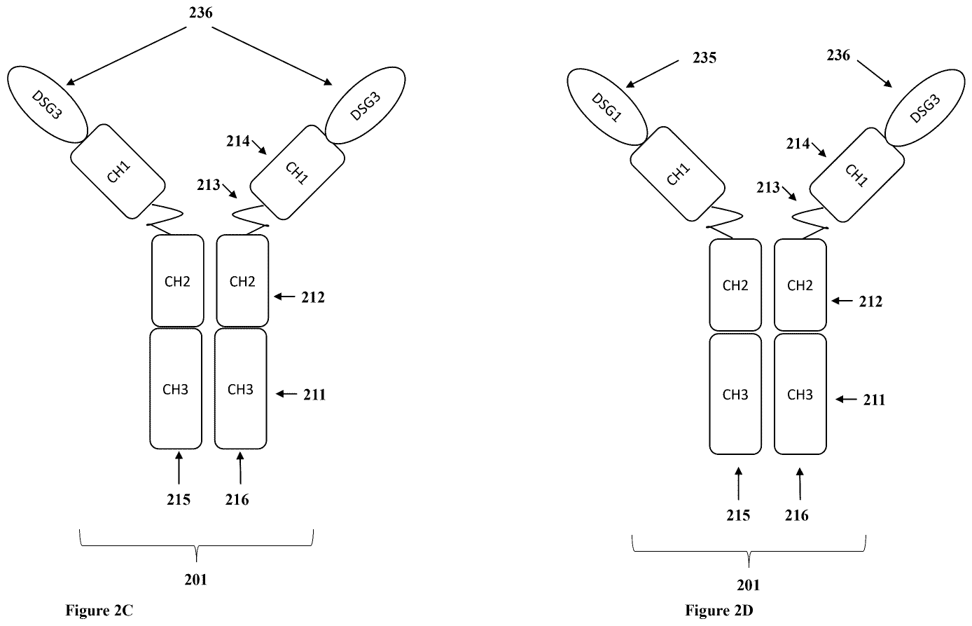

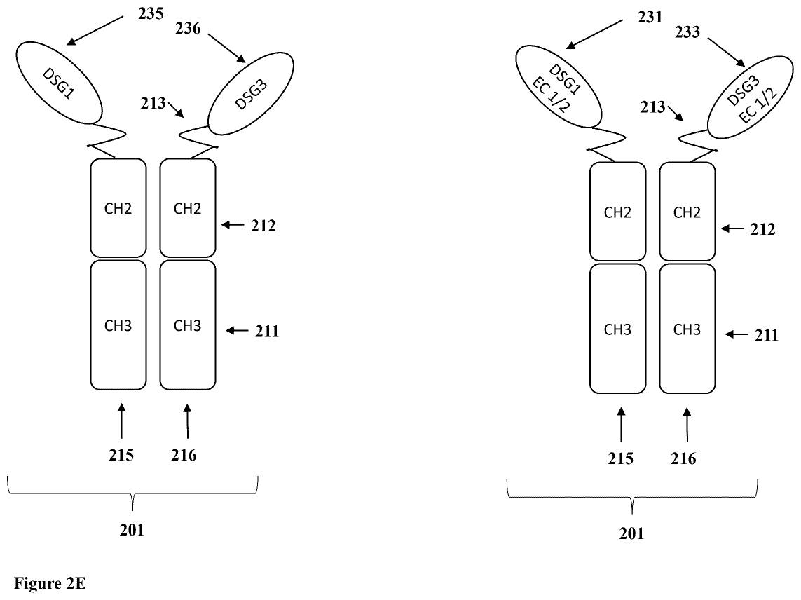

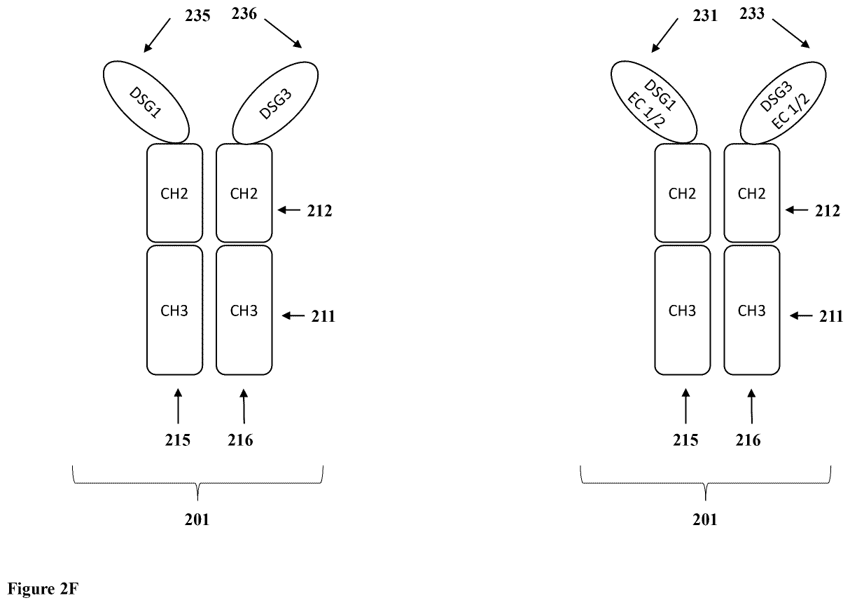

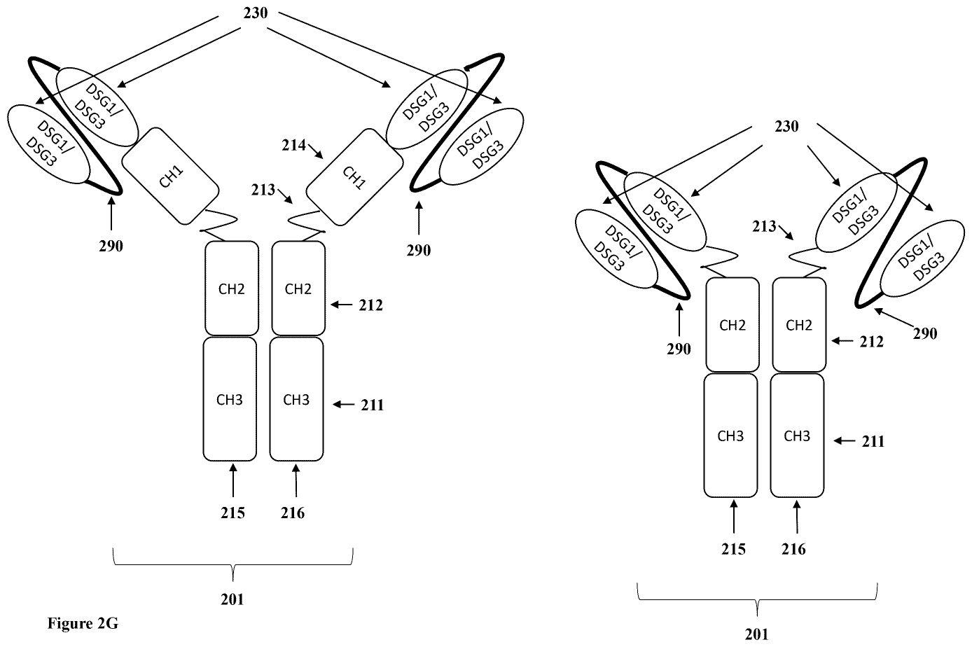

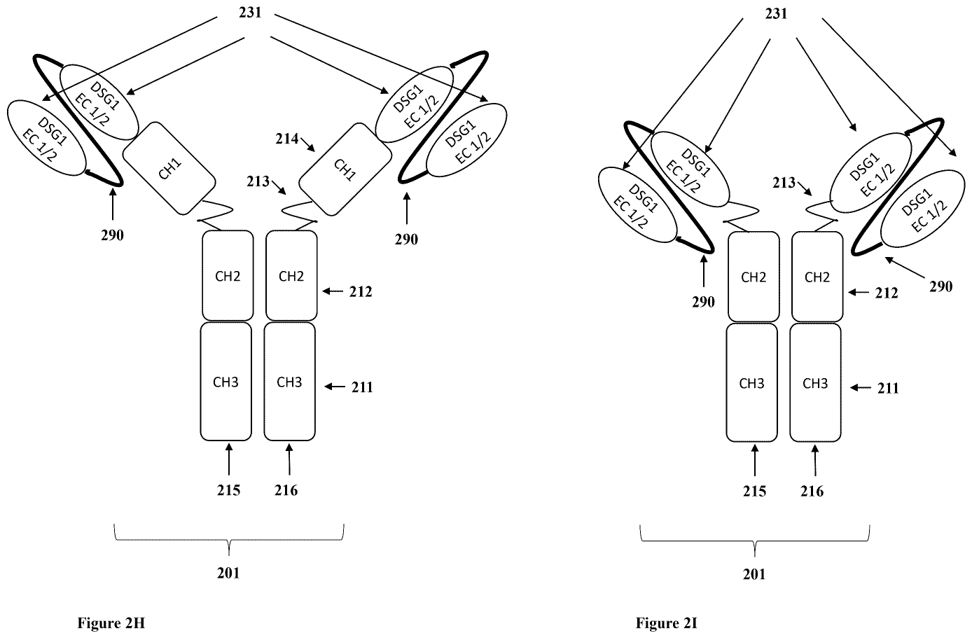

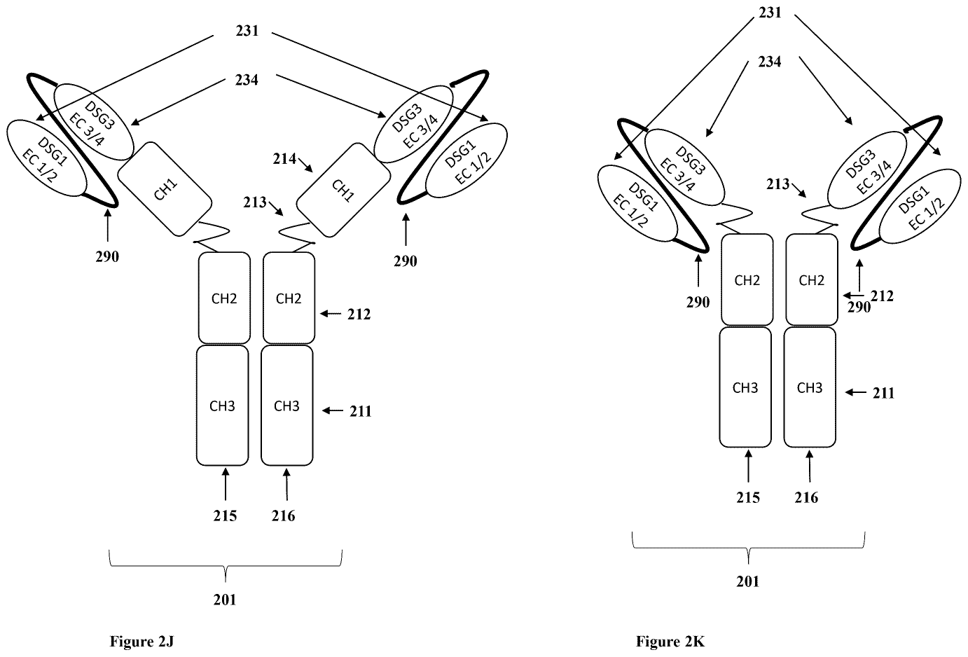

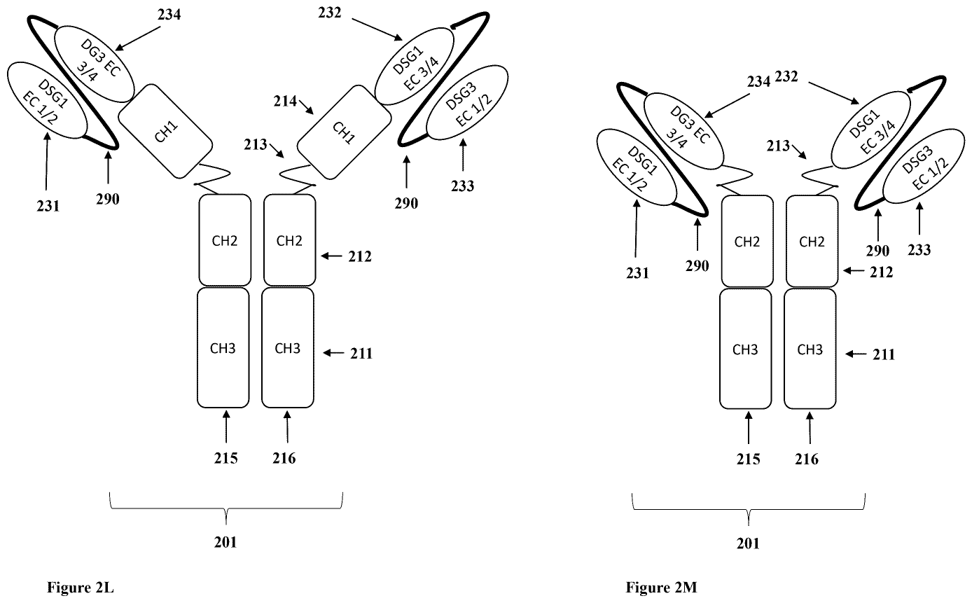

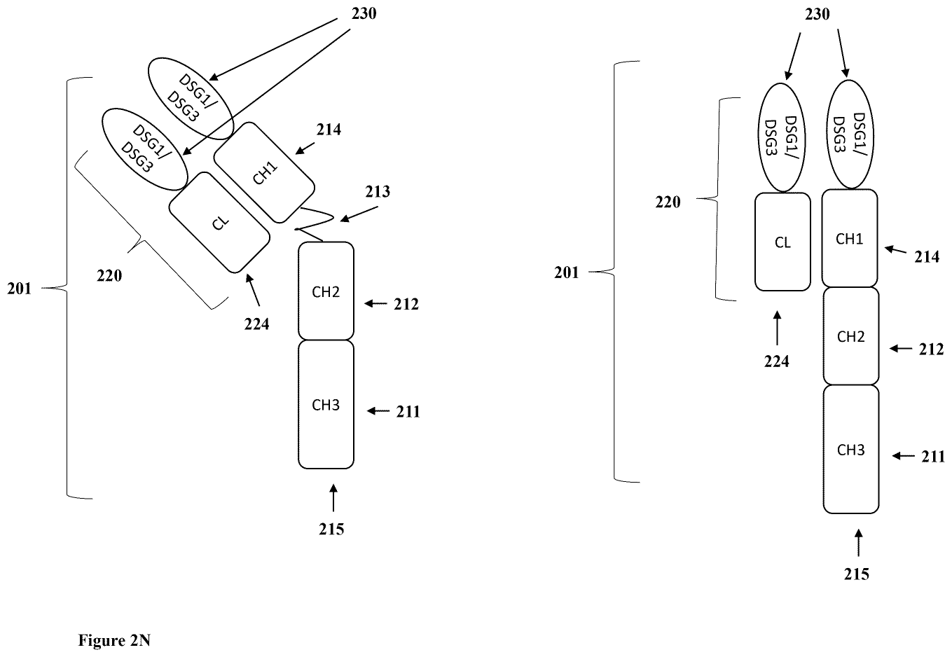

FIGS. 2A-2N show some embodiments of the invention in which only two chains are combined. In FIGS. 2A-2F, protein complex 201 comprises 2 polypeptide chains which specifically are two heavy chains. Heavy chains 215 and 216 can optionally include a hinge domain 213, CH2 212, CH3 211 and/or CH1 214 domain. In this embodiment, the heavy chain hinge 213, CH2 212, and CH3 211 all dimerizes via disulfide bonds. Only one of these 3 domains is needed for dimerization and other dimerization domains are also envisioned. The extracellular domain of either DSG1 or DSG3 230 is included. Protein complex 201 is also envisioned with only a fragment of the DSG1 or DSG3 extracellular domain, such as fragment 231 comprising just EC1 and EC2 of DSG1, fragment 232 comprising just EC3 and EC4 of DSG1, fragment 233 comprising just EC1 and EC2 of DSG1, and fragment 234 comprising just EC3 and EC4 of DSG3. A protein complex 201 with two different fragments either from the same protein or from each of the two proteins (such as fragment 231 on one chain and fragment 233 on the other) is also envisioned. Although these specific fragments are depicted here any fragment of the extracellular domain of either protein may be used in any combination.

FIG. 2B shows the molecule without CH2 domain 212, CH3 domain 211 or CH1 domain 214. Molecules lacking both hinge 213 and CH1 domain 214 are also depicted. Combinations lacking two of these domains are also envisioned, with or without hinge 213 (FIG. 2B). In place of the variable region each chain has a fragment 230 from the extracellular portion of DSG1 and/or DSG3. Although it is not depicted, it will be understood by a skilled artisan that any fragment from the extracellular portion of DSG1 and/or DSG3 can also be used. Each chain can be generated to have the same DSG protein, fragment or variant (FIG. 2C) or different subunits (FIG. 2D). For ease the extracellular domain of DSG1 235 and the extracellular domain of DSG3 236 are depicted, but it will be understood that fragments or variants of these domains can also be used. When two different subunits are employed, it is advantageous to design the molecule such that predominantly heterodimers of 215 and 216 are formed and not homodimers. The same is true of forming heterodimers of chain 115 and chain 116 in FIG. 1. The same applies to FIG. 2A. There are numerous technologies known in the art for designing mutations in the CH3/CH2 domains, such as Knobs-in-Holes, DuoBodies, etc., that inhibit homodimerization and promote heterodimerization. Any such technology may be employed. Removal of the CH1 with direct conjugation to the hinge region (FIG. 2E) or additional removal of the hinge with direct conjugation to the CH2 (FIG. 2F) is also possible. It will be understood that for all figures, when an extracellular domain is depicted it is intended also include all fragments, variant and mutants of that extracellular domain.

In FIG. 2G alternative configurations comprising two heavy chains are shown. Instead of containing a single fragment 230 in place of the variable region, two tandem fragments 230 are used. These fragments may be separated by optional linker 290. This configuration is similar in structure to a single chain antibody in which the heavy and light chain variable domains are on a single peptide and is essentially equivalent to the molecule shown in FIG. 1D. Heavy chains 215 and 216 can optionally include a hinge 213, CH2 212, CH3 211 and/or CH1 214 domain. Dimerization is as described above. For simplicity an example containing all three CH domains is shown as is an example lacking the CH1 domain. Molecules lacking the hinge, CH2 or CH3 domain or lacking any two/three of these domains are also envisioned (so long as at least one region of dimerization is retained). It will be understood that fragment 230 can be from either DSG1 or DSG3 and can include the whole extracellular domain or only a portion or a variant thereof. Thus, a repeat of two of the same protein/fragment can be inserted on a single chain (FIG. 2H-I, two DSG1 EC 1/2 fragments 231) or two different proteins/fragments can be combined on one chain (FIG. 2.J-K, a DSG1 EC 1/2 fragment 231 and a DSG3 EC 3/4 fragment 234). Of course, the heavy chains need not be identical as various technologies may be used to favor heterodimerization over homodimerization (FIG. 2L-2M, DSG1 EC 1/2 fragment 231 and DSG3 EC 3/4 fragment 234 on one chain and an DSG3 EC 1/2 fragment 233 and DGS1 EC 3/4 fragment 232 on the other chain). As before, CH1 domain 214 can be included (FIG. 2H, 2J, 2L) or excluded (FIG. 2L, 2K, 2M) and the same is true for hinge 213, CH2 212 and/or CH3 211 so long as one dimerization domains (e.g., hinge, CH2, CH3) remains.

In FIG. 2N, protein complex 201 comprises 2 polypeptide chains which specifically are a heavy chain 215 and a light chain 220. In such an embodiment the dimerization domains are CH1 domain 214 and CL domain 224. Heavy chain 215 may optionally include CH3 domain 211, CH2 domain 212 and/or hinge region 213. Absence of the hinge/CH2/CH3 domains is one option for eliminating homodimerization of two heavy chains 215. Alternatively, cysteine substitutions/mutations (to serine or glutamine for example) may be introduced into the hinge or one of the mutations in the CH2/CH3 regions that promote heterodimerization and inhibit homodimerization may be employed. In place of the variable region each chain has a fragment 230 from the extracellular portion, or any fragment or variant thereof, of DSG1 or DSG3.

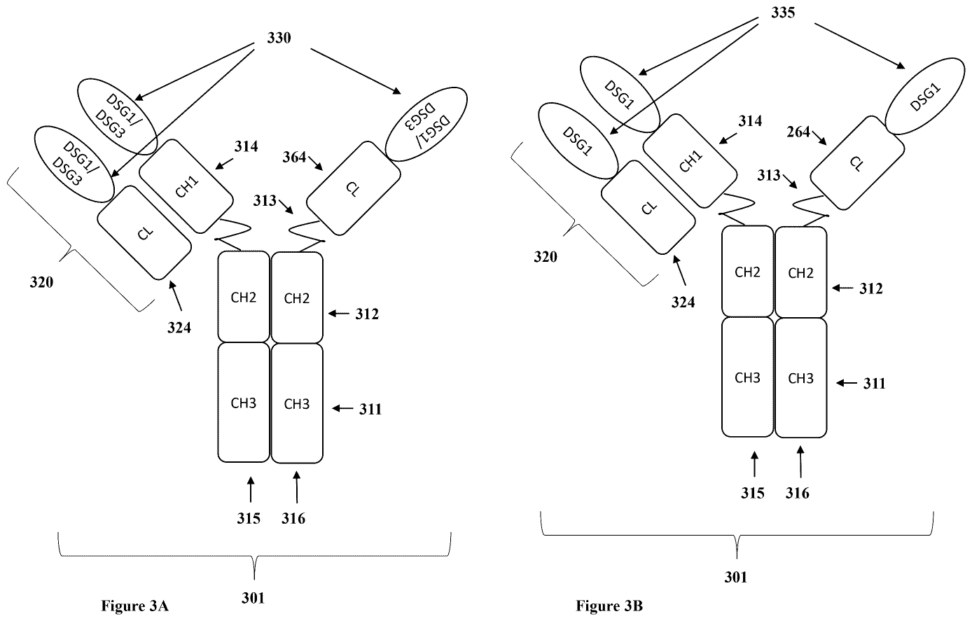

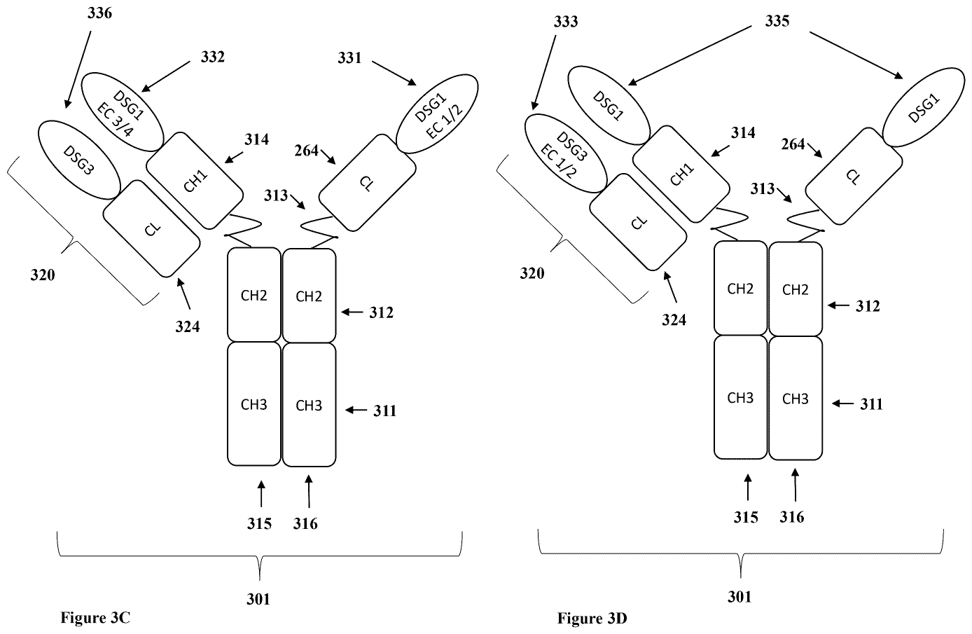

The creation of a protein complex 301, which has three chains, a heavy chain 315, a heavy chain 316 and a light chain 320 is also envisioned (FIG. 3A-3D). FIG. 3A shows one possible embodiment in which heavy chain 316 comprises a CL domain 364 in place of a CH1 domain. The hereinabove described methods of ensuring a 315/316 heterodimer can be employed. Heavy chains 315 and 316 may optionally include CH3 domain 311, CH2 domain 312 and/or hinge region 313 or may employ a different dimerization domain. CL domain 324 within light chain 320 can only dimerize with CH1 domain 314 within heavy chain 315. In place of the variable region each chain has a fragment 330 from the extracellular portion of human Desmoglein ⅓. The three chains can all contain the same fragment (for example the DSG1 extracellular domain 335, FIG. 3B), all three chains can contain different fragments (for example DSG3 336, DSG1 EC 1/2 fragment 331 and DSG1 3/4 fragment 332, FIG. 3C), or the three chains can contain two different fragments in which one is repeated (for example DSG1 335 and DSG3 EC 1/2 fragment 333, FIG. 3D). It will be understood by a skilled artisan that FIG. 3D could also have the two identical fragments as the light chain and either of the heavy chains, thus there are 3 different configurations to this embodiment.

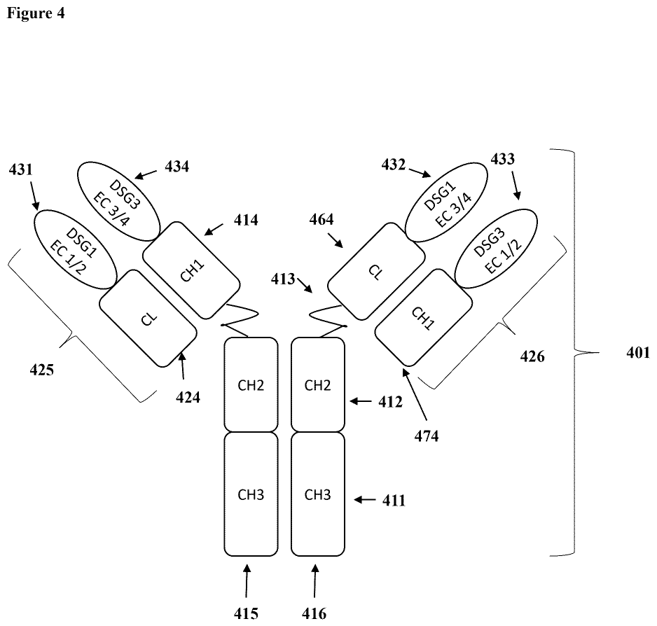

This configuration, with one of the heavy chains comprising a CL domain in place of a CH1 domain can also allow for the formation of the protein complex with four different fragments. Similar to the protein complex of FIG. 1B, protein complex 401 depicted in FIG. 4 has four different fragments on each chain. In this embodiment, the fragments DSG1 EC 1/2 431, DSG3 EC 3/4 434, DSG1 EC 3/4 432 and DSG3 EC 1/2 433 are employed but a skilled artisan will appreciate that any four fragments can be used. Embodiments are also envisioned in which different fragment from the same protein can be on different chains. In this embodiment, the second light chain 426 contains a CH1 domain 474 so that it can dimerize with the CL domain 464 in heavy chain 416. Heavy chain 415 will contain a CH1 domain 414 and light chain 425 will contain a CL domain 424. This ensures that chain 425 can dimerize only with chain 415 and chain 426 can dimerize only with chain 416. As described hereinabove, mutations in the optional CH2 domains 412 and the CH3 domains 413 can be employed to promote heterodimerization of chains 415 and 416. Hinge region 413, CH2 domain 412 and CH3 domain 411 are all used here as dimerization domains between the two heavy chains, though any dimerization domain (other than CH1/CL) can be employed.

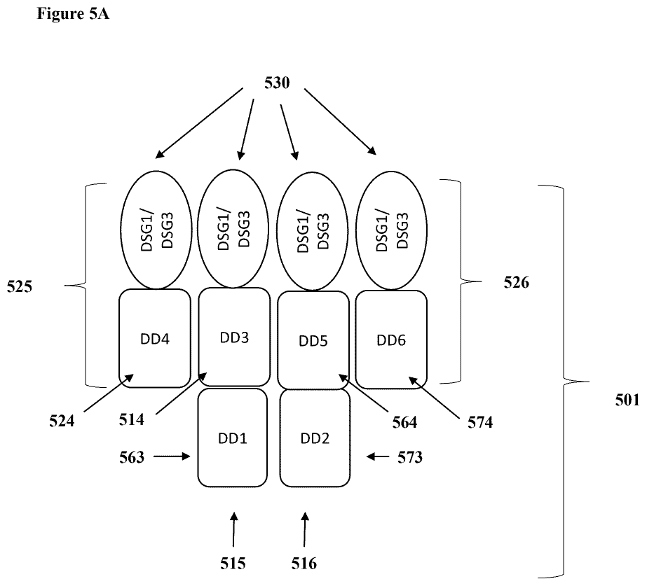

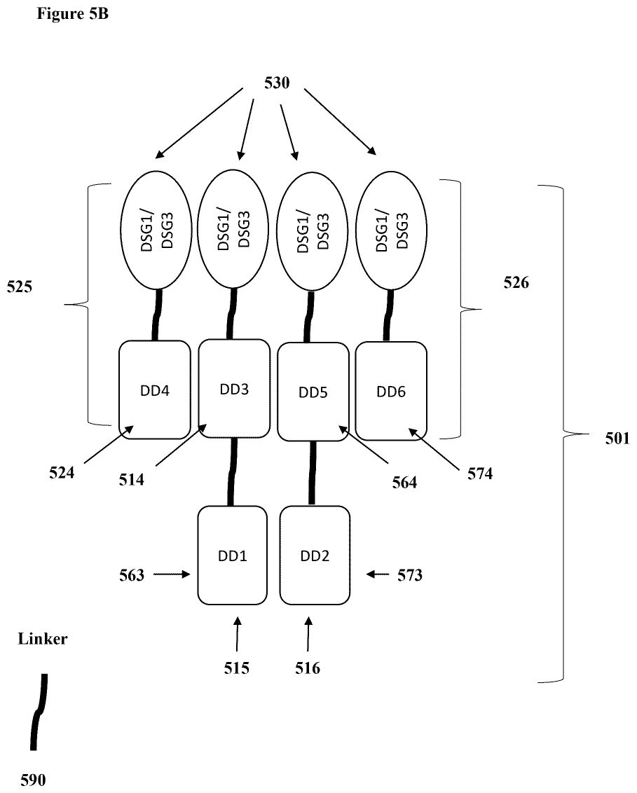

In the above-described embodiments, an immunoglobulin backbone is depicted and described, but it will be understood by a skilled artisan that by selecting other dimerization domains similar molecules can be generated. FIGS. 5A-5B show a generic protein complex 501. In FIG. 5A the first chain 515 contains a first dimerization domain (DD1) 563 which can dimerize specifically with a second dimerization domain (DD2) 573 of second chain 516. Chain 515 further comprises a third dimerization domain (DD3) 514 which can dimerize specifically with a fourth dimerization domain (DD4) 524 of third chain 525. Chain 516 further comprises a fifth dimerization domain (DD5) 564 which can dimerize specifically with a sixth dimerization domain (DD6) 574 of fourth chain 526. Each of the four chains also comprises a fragment 530 of a human protein target of PV/PF autoantibodies. These can be all the same fragment with the same amino acid sequence, or they can be different sequences (either from the same protein or from different proteins) or variants/derivatives or the proteins or fragments.

FIG. 5B shows an alternative embodiment to FIG. 5A in which each distinct domain is separated by a linker. It will be understood by a skilled artisan that all of these linkers are optional, and that combination of linkers is envisioned. It will be further understood that the configurations of FIG. 5B also could employ linkers between any or all of the various domains/fragments. Similarly, linkers can be inserted between the CH1 domain, hinge region, CH2 domain, CH3 domain and/or the DSG1/3 fragments. Thus, the linkers shown in FIG. 5B can be extrapolated to the same positions in the immunoglobulin backbone molecules (FIG. 1A-4).

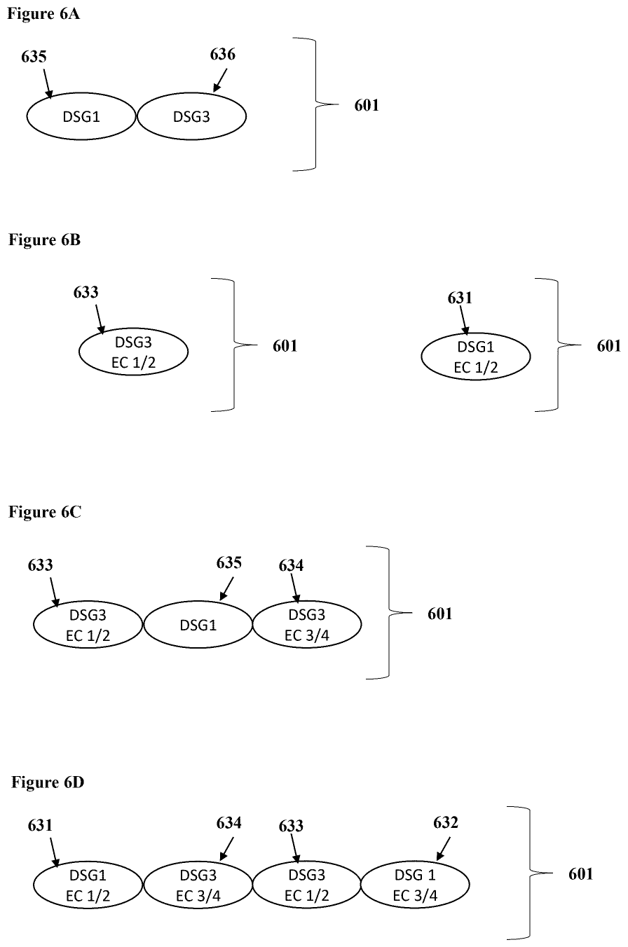

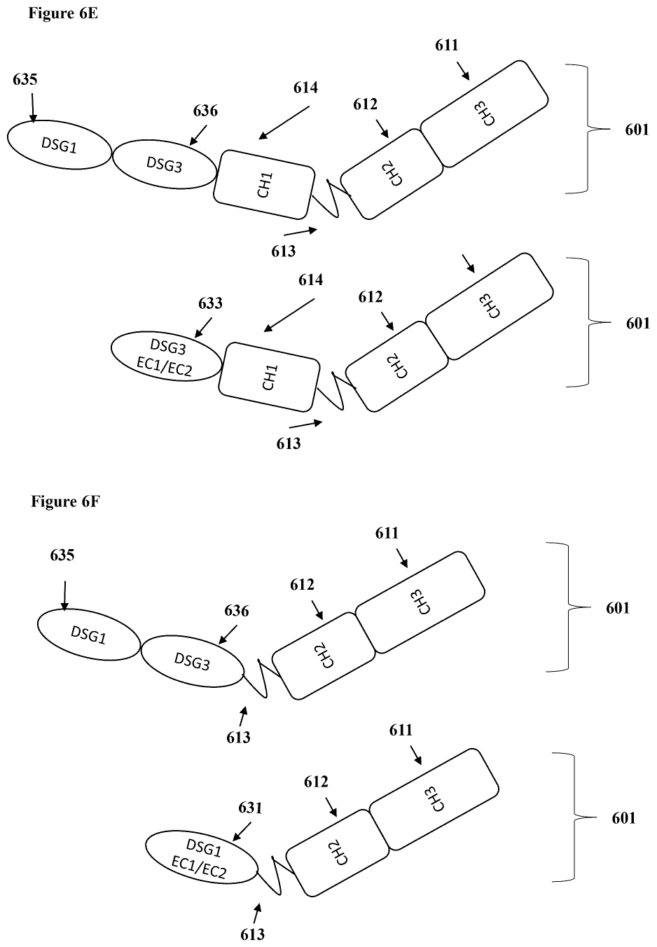

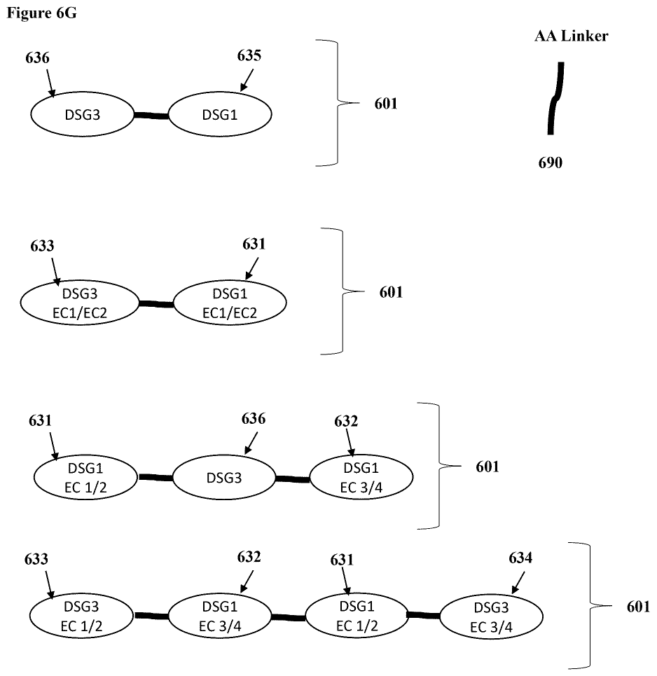

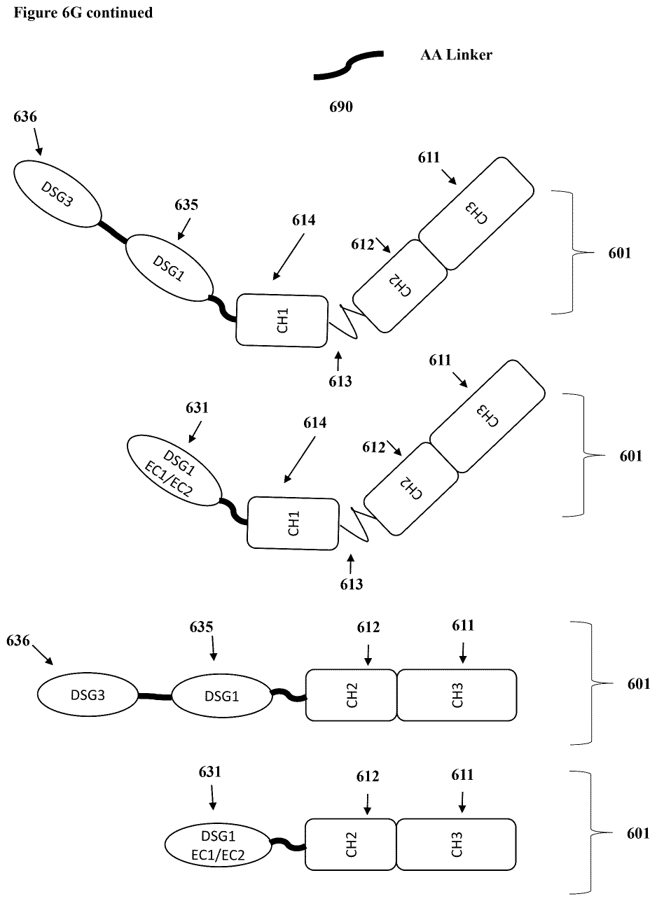

In FIG. 6A-6F single chain embodiments of the invention are depicted. FIG. 6A shows a single chain fusion protein 601 containing the DSG1 extracellular domain 635 and the DSG3 extracellular domain 636. FIG. 6B shows a single chain comprising only a fragment of DSG1 or a fragment of DSG3. For simplicity a fragment comprising just the EC1 and EC2 of DSG1 631 and a fragment comprising just the EC1 and EC2 of DSG3 633 are shown, although it will be understood that any fragment of the extracellular domain may be used. It will further be understood that any permutation of FIGS. 6A and 6B may generated such that two fragments from different proteins are in the same single chain, two different fragments can be used. Specifically fragments from two different proteins can be used. FIGS. 6C and 6D show a similar embodiment but containing 3 and 4 fragments from different proteins respectively. It will be understood that any fragment or the complete extracellular domain can be used. As shown in FIG. 6E, the single chain can also contain a heavy chain constant region with at least a CH3 domain 611 or CH2 domain 612 and optionally CH1 domain 614, hinge region 613 and/or CH2 domain 612 or CH3 domain 611. FIG. 6F shows the embodiment in which the CH1 domain has been removed. Finally, amino acid linkers can be used to separate any of the domains of the single chain. FIG. 6G depicts embodiments, with 2, 3, or 4 fragments all separated by linkers 690 as well as embodiments in which linker 690 also separates the C-terminal fragment 635 from CH1 domain 614 or CH2 domain 612. It will be understood that while the linker is shown also replacing the hinge region this is not required and the hinge could be retained with the linker linking C-terminal fragment 635 to it. Although, no linkers are depicted separating the CH1 domain 614, the hinge region 613, the CH2 domain 612 and the CH3 domain 611, it will be understood by a skilled artisan that any or all of these domains could be separated by linkers. Further, it will be understood that these various linkers can all contain the same sequence or can be made of different amino acid sequences.

### Example 2

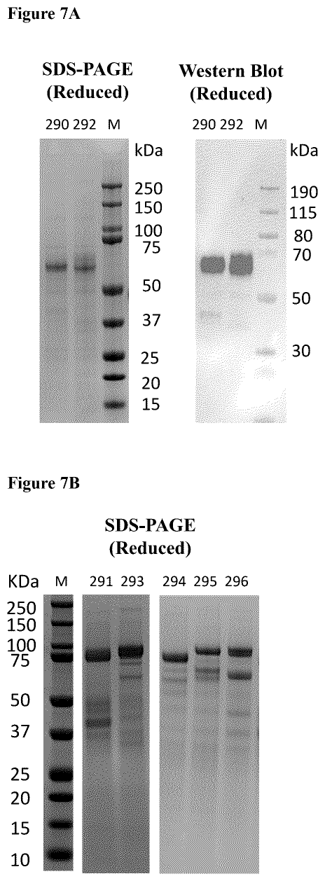

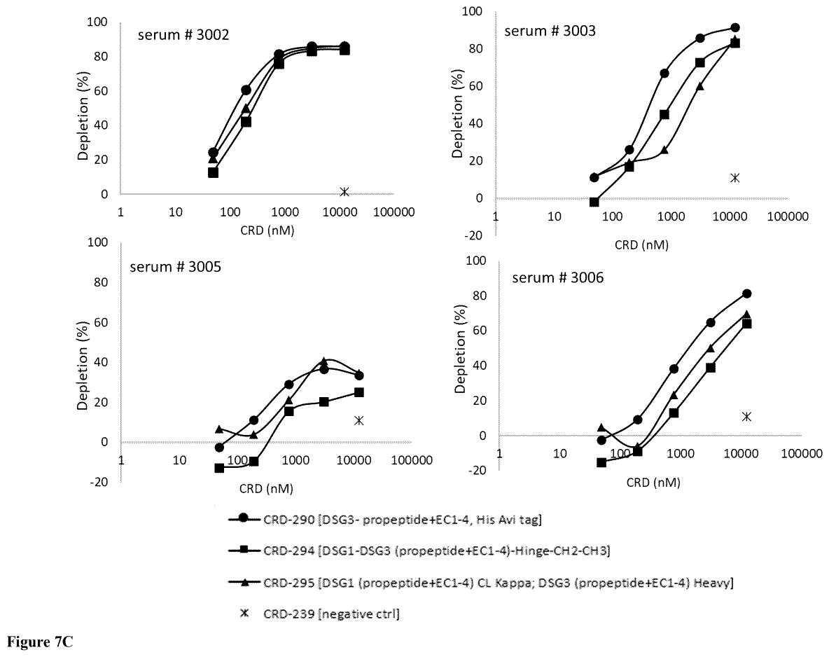

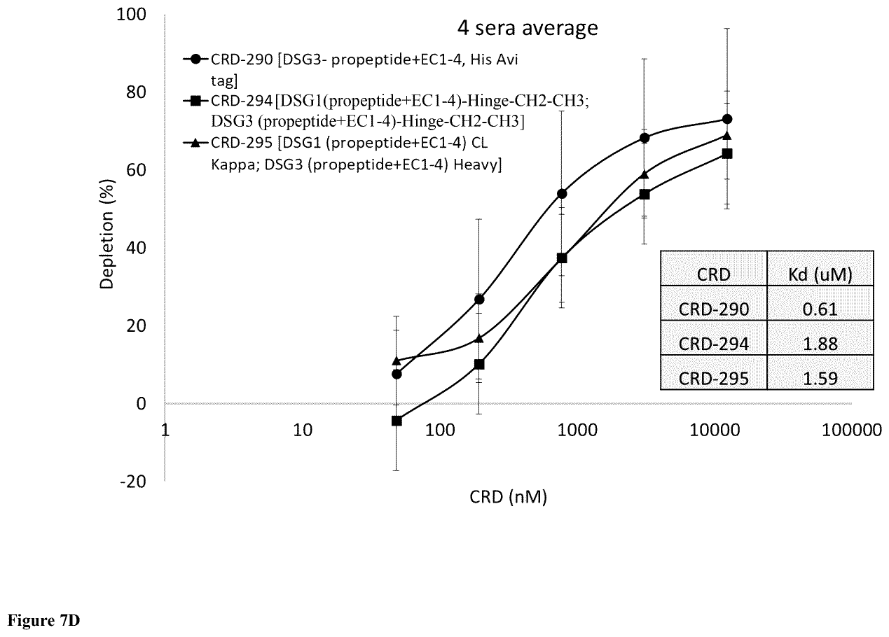

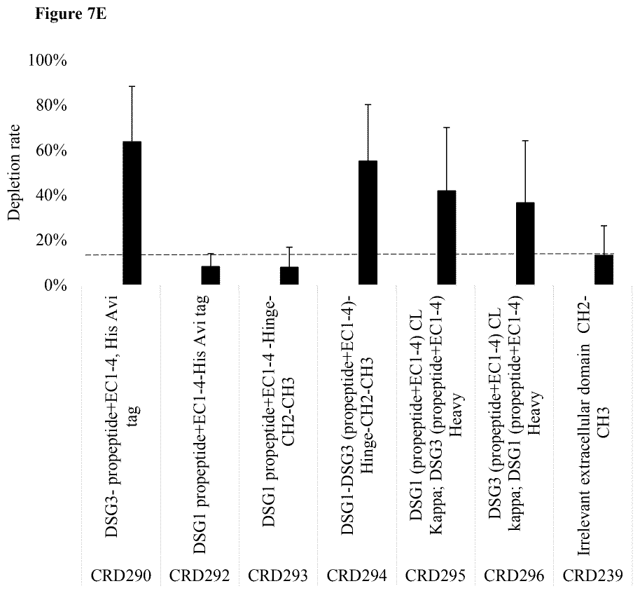

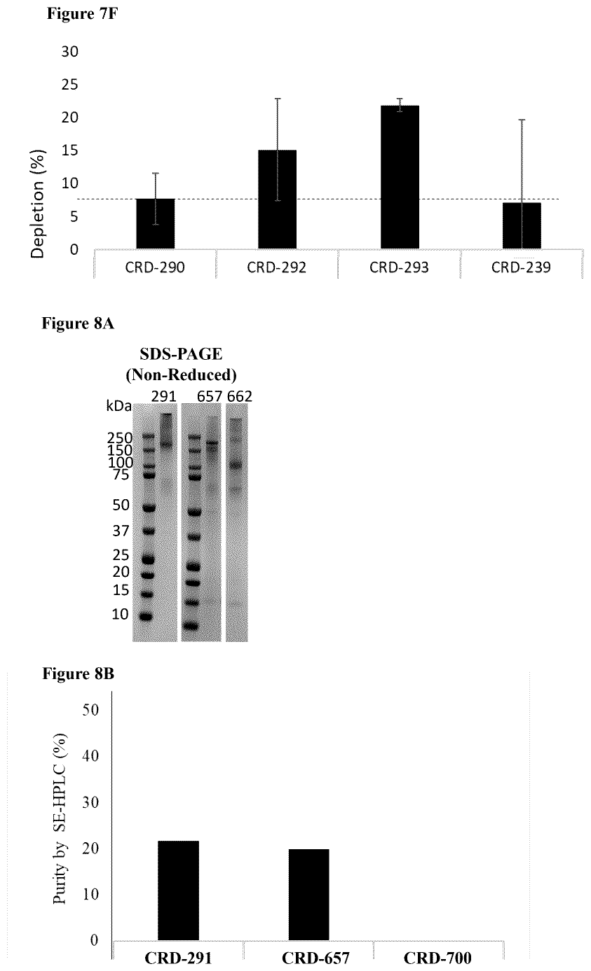

In an initial batch, the full extracellular domain of DSG1 (SEQ ID NO: 1, lacking signal peptide but including the prodomain) or the full extracellular domain of DSG3 (SEQ ID NO: 3, lacking signal peptide but including the prodomain) was transiently expressed in CHO cells at a scale of 250 ml (FIG. 7A). The molecules were generated with an 8X His tag and an AVI tag (SEQ ID NO: 5-6, for DSG1 and DSG3 respectively) for easier protein purification. A positively charged linker EAAAKEAAAK (SEQ ID NO: 65) was used to separate the tags from the C-terminus of the extracellular domains.

Additional molecules in which the extracellular domains were combined with an IgG hinge and CH2 and CH3 domains were also generated (SEQ ID NO: 5 and 6, for DSG1 and DSG3 respectively). A (GGGGS) 3 linker (SEQ ID NO: 66 repeated 3 times) was used to link the C-terminus of the extracellular domain to the hinge. Similarly, molecules in which the extracellular domains were combined with the CL Kappa domain were also generated (SEQ ID NO: 7; and 8, for DSG1 and DSG3 respectively). Molecules in which the Fc regions were mutated so as to favor heterodimerization over homodimerization (knobs-in-holes approach: T366W mutation within the CH3 domain of the DSG3 containing molecule, SEQ ID NO: 9; and T366S/L368A/Y407V triple mutation within the CH3 domain of the DSG1 containing molecule, SEQ ID NO: 10) were also generated. The two heavy chains intended to heterodimerized were expressed together, as were the DSG1 heavy chain with the DSG3 light chain and the DSG3 heavy chain with the DSG1 light chain (FIG. 7B). The produced molecules are summarized in Table 2, which provides the identifier used herein throughout, expected molecular weight (MW), expected isoelectric point (pI, (M−1*cm−1)), expected extinction coefficient (EC) and actual yield of the various molecules.

TABLE 2
Batch I of molecules of the invention

| Identifier | | MW | | | Yield |
|---|---|---|---|---|---|
| SEQ ID: ( ) | Molecules Description | (kDa) | pI | EC | (mg/L) |
| CRD-290 | DSG3- propeptide + EC1-4, His Avi tag | 57.7 | 5.4 | 0.77 | 198 |
| (6) | | | | | |
| CRD-291 | DSG3-propeptide + EC1-4- Hinge-CH2-CH3 | 161.1 | 5.5 | 0.93 | 162 |
| (8) | | | | | |
| CRD-292 | DSG1 propeptide + EC1-4-His Avi tag | 57.4 | 5.5 | 0.85 | 201 |
| (5) | | | | | |
| CRD-293 | DSG1 propeptide + EC1-4 -Hinge-CH2-CH3 | 160.4 | 5.6 | 0.99 | 181 |
| (7) | | | | | |
| CRD-294 | DSG1 propeptide + EC1-4 -Hinge-CH2-CH3 | 160.7 | 5.6 | 0.98 | 326 |
| (9 + 10) | Heavy Chain KIH hole | | | | |
| | DSG3 propeptide + EC1-4 -Hinge-CH2-CH3 | | | | |
| | Heavy Chain KIH knob | | | | |
| CRD-295 | DSG1 (propeptide + EC1-4) CL Kappa; | 312.4 | 5.6 | 0.90 | 186 |
| (11 + 12) | DSG3 (propeptide + EC1-4) Heavy | | | | |
| CRD-296 | DSG3 (propeptide + EC1-4) CL kappa; | 312.4 | 5.6 | 0.90 | 158 |
| (13 + 14) | DSG1 (propeptide + EC1-4) Heavy | | | | |

Next, the binding capacity of the various molecules to actual pathogenic antibodies was determined in PV patients' serum samples. Four human sera samples positive for anti-DSG3 IgG antibodies (titer>20 RU/mL) were used to examine molecule binding. Autoantibody titer was determined using Euroimmun ELISAs. The molecules were biotinylated and attached to avidin coated Sepharose beads, and sera samples were separately incubated with increasing concentrations of the various DSG-3 containing molecules. As can be seen in FIG. 7C, all the molecules that include the DSG-3 extracellular domain were able to bind the anti-DSG3 antibodies, regardless of whether the DSG-3 extracellular domain was present on the heavy chain or light chain or whether the DSG-1 extracellular domain was present or not. However, while in 3 of the samples a greater than 80% depletion of antibodies was possible in one subject only about 40% depletion could be achieved. CRD-239, an Fc-fusion molecule containing the extracellular domain of an irrelevant protein, was used as a negative control for both the antigen side and the Fc non-specific binding, and had no effect on autoantibody levels showing that the binding was antigen specific and Fc independent. The average depletion of all the sera with respect to anti-DSG-3 were fairly comparable (FIG. 7D).

The 0.77 μM concentration was found to produce 90% or more of the binding for all the tested molecules and therefore was selected for further analysis. In addition to testing the DSG3 containing molecules, the DSG1 only molecules were also included as negative controls. As expected, all the DSG3 containing molecules, but none of the DSG1 only molecules, were able to deplete anti-DSG3 antibodies for all patient sera (FIG. 7E). The molecules with DSG3 fused to the heavy chain produced better depletion than the molecule with DSG3 fused to the light chain.

A single human serum samples positive for anti-DSG1 IgG antibodies (titer>20 RU/mL) was also used to test the ability of the DSG1 molecules to deplete autoantibodies. The 0.77 μM reference concentration was again used and the DSG1 containing molecules were able to deplete autoantibody levels (FIG. 7F). The assay was repeated multiple times on the single sample. Interestingly, the heavy chain containing molecule was actually superior to the molecule without a dimerization domain. Further, the DSG3 containing molecules were comparable to the negative control.

### Example 3

Notably molecules that included the dimerization domains produced slightly reduced yields, although the heavy chain heterodimer produced a surprisingly high yield (see Table 2). However, when these transient expressions were repeated at a smaller scale (20 ml scale) both the DSG1 (see Table 3) and the DSG3 (see Table 4) Fc fusion molecules produced greatly reduced yields. These analyses were performed on denaturing gels, however, on non-reduced gels a smear of very high weight aggregates was observed. Similarly, visual observation of the molecules showed high levels of inter-molecule aggregation. This is not surprising as the purpose of Desmogleins in nature is to produce tight junctions. The adhesion of the molecules was causing clumping, poor isolation and low yields. In order to remedy this problem, various variants (mutants) of DSG1 and DSG3 were generated. The mutations were designed to decrease homo- and hetero-adhesion. Truncations containing only some of the 4 EC domains were also investigated (DSG1 EC 1/2/3, DSG1 EC 2/3/4, DSG1 EC 1/2, DSG1 EC 3/4, DSG3 EC 1/2/3, DSG3 EC 2/3/4, DSG3 EC 1/2, DSG3 EC 3/4, SEQ ID NO: 57-64). A summary of the DSG1 containing molecules and their characteristics is provided in Table 3. A summary of the DSG3 containing molecules and their characteristics is provided in Table 4.

TABLE 3
Batch II of DSG1 molecules of the invention (mutation numbering
is with respect to the full DSG1 protein including the prodomain)

| Identifier | | | | | |
|---|---|---|---|---|---|
| SEQ ID | | MW | | | Yield |
| NO: ( ) | Molecules Description | (kDa) | pI | EC | (mg/L) |
| CRD-293 | DSG1-propeptide, EC1, EC2, EC3, EC4- | 160.4 | 5.7 | 0.99 | 51 |
| (7) | CH2-CH3 | | | | |
| CRD-666 | DSG1-propeptide, EC1 (W28A), EC2, EC3, | 161.5 | 5.7 | 0.91 | 80 |
| (15) | EC4-CH2-CH3 | | | | |
| CRD-667 | DSG1-propeptide, EC1 (D53T), EC2, EC3, | 161.7 | 5.8 | 0.98 | 52 |
| (16) | EC4-CH2-CH3 | | | | |
| CRD-668 | DSG1-propeptide, EC1 (E115S), EC2, EC3, | 161.6 | 5.8 | 0.98 | 44 |
| (17) | EC4-CH2-CH3 | | | | |
| CRD-669 | DSG1-propeptide, EC1 (W28A, D53T, | 161.4 | 5.9 | 0.91 | 51 |
| (18) | E115S), EC2, EC3, EC4-CH2-CH3 | | | | |
| CRD-670 | DSG1-propeptide, EC1 (W28A, A106I), | 161.6 | 5.7 | 0.91 | 104 |
| (19) | EC2, EC3, EC4-CH2-CH3 | | | | |
| CRD-671 | DSG1-propeptide, EC1 (K43A, R44A), EC2, | 161.4 | 5.6 | 0.98 | 120 |
| (20) | EC3, EC4-CH2-CH3 | | | | |
| CRD-672 | DSG1-propeptide, EC1 (R36A, K43A, R44A, | 161.1 | 5.6 | 0.98 | 90 |
| (21) | R123A), EC2, EC3, EC4-CH2-CH3 | | | | |
| CRD-673 | DSG1-propeptide, EC1 (C35V, C54A, | 161.6 | 5.7 | 0.98 | 68 |
| (22) | C104V), EC2 (C230A, C232V), EC3, EC4 | | | | |
| | (C452S)-CH2-CH3 | | | | |
| CRD-674 | DSG1-propeptide, EC1 (C35V, C54A, | 161.6 | 5.7 | 0.98 | 73 |
| (23) | C104V) EC2 (C230A, C232V), EC3, EC4- | | | | |
| | CH2-CH3 | | | | |
| CRD-701 | DSG1-pro-peptide, EC1 (C35S, C54S, | 161.6 | 5.7 | 0.98 | 39 |
| (56) | C104S), EC2 (C230S, C232S), EC3, EC4- | | | | |
| | CH2-CH3 | | | | |
| CRD-675 | DSG1-propeptide, EC1 (E95G, D129G), EC2 | 161.4 | 6.0 | 0.98 | 55 |
| (24) | (D162G), EC3, EC4-CH2-CH3 | | | | |
| CRD-676 | DSG1-propeptide, EC1, EC2 (E205G, | 161.4 | 6.0 | 0.98 | 30 |
| (25) | D241G), EC3 (D273G), EC4-CH2-CH3 | | | | |
| CRD-677 | DSG1-propeptide, EC1, EC2, EC3 (E257G, | 161.3 | 6.0 | 0.98 | 49 |
| (26) | E317G, E358G), EC4-CH2-CH3 | | | | |
| CRD-678 | DSG1-propeptide, EC1, EC2, EC3-CH2-CH3 | 137.4 | 5.6 | 1.06 | 51 |
| (27) | | | | | |
| CRD-679 | DSG1-propeptide, EC1, EC2-CH2-CH3 | 111.2 | 6.2 | 1.04 | 80 |
| (28) | | | | | |
| CRD-680 | DSG1-propeptide, EC2, EC3, EC4-CH2-CH3 | 137.2 | 5.8 | 1.01 | 52 |
| (29) | | | | | |
| CRD-681 | DSG1-propeptide, EC3, EC4-CH2-CH3 | 112.3 | 7.3 | 1.15 | 44 |
| (30) | | | | | |
| CRD-923 | DSG1-propeptide, EC1 (C35V, C54A, | 156.2 | 5.7 | 1.8 | 104 |
| (31) | C104V), EC2 (C230A, C232V)-CH2-CH3 | | | | |
| | (LALA) | | | | |

TABLE 4
Batch II of DSG3 molecules of the invention (mutation numbering
is with respect to the full DSG1 protein including the prodomain)

| | | MW | | | Yield |
|---|---|---|---|---|---|
| Identifier | Molecules Description | (kDa) | pI | EC | (mg/L) |
| CRD-291 | DSG3-propeptide, EC1, EC2, EC3, EC4-CH2- | 161.1 | 5.7 | 0.93 | 25 |
| (8) | CH3 | | | | |
| CRD-650 | DSG3-propeptide, EC1 (W28A), EC2, EC3, | 162.1 | 5.6 | 0.86 | 22 |
| (32) | EC4-CH2-CH3 | | | | |
| CRD-652 | DSG3-propeptide, EC1 (E115S), EC2, EC3, | 162.3 | 5.7 | 0.93 | 107 |
| (33) | EC4-CH2-CH3 | | | | |
| CRD-653 | DSG3-propeptide, EC1 (W28A, D53T, | 162.0 | 5.7 | 0.86 | 80 |
| (34) | E115S), EC2, EC3, EC4-CH2-CH3 | | | | |
| CRD-654 | DSG3-propeptide, EC1 (W28A, A106I), EC2, | 162.2 | 5.6 | 0.86 | 70 |
| (35) | EC3, EC4-CH2-CH3 | | | | |
| CRD-655 | DSG3-propeptide, EC1 (K43A, R44A), EC2, | 162.1 | 5.6 | 0.93 | 175 |
| (36) | EC3, EC4-CH2-CH3 | | | | |
| CRD-656 | DSG3-propeptide, EC1 (R36A, K43A, R44A, | 161.8 | 5.5 | 0.93 | 87 |
| (37) | K123A), EC2, EC3, EC4-CH2-CH3 | | | | |
| CRD-657 | DSG3-propeptide, EC1 (C35V, C104V), EC2 | 162.3 | 5.6 | 0.92 | 130 |
| (38) | (C228A, C230V), EC3, EC4-CH2-CH3 | | | | |
| CRD-700 | DSG3-pro-peptide, EC1 (C35S, C104S), EC2 | 162.2 | 5.6 | 0.92 | 24 |
| (55) | (C228S, C230S), EC3, EC4-CH2-CH3 | | | | |
| CRD-658 | DSG3-propeptide, EC1 (E95G, D129G), EC2 | 162.0 | 5.8 | 0.93 | 17 |
| (39) | (D162G), EC3, EC4-CH2-CH3 | | | | |
| CRD-659 | DSG3-propeptide, EC1, EC2 (E205G, D239G), | 162.0 | 5.8 | 0.93 | 18 |
| (40) | EC3 (D271G), EC4-CH2-CH3 | | | | |
| CRD-660 | DSG3-propeptide, EC1, EC2, EC3 (E255G, | 161.9 | 5.9 | 0.93 | 23 |
| (41) | E315G, E356G), EC4-CH2-CH3 | | | | |
| CRD-661 | DSG3-propeptide, EC1, EC2, EC3-CH2-CH3 | 136.7 | 5.5 | 0.97 | 49 |
| (42) | | | | | |
| CRD-662 | DSG3-propeptide, EC1, EC2-CH2-CH3 | 110.2 | 5.8 | 0.86 | 99 |
| (43) | | | | | |
| CRD-663 | DSG3-propeptide, EC2, EC3, EC4-CH2-CH3 | 138.1 | 5.7 | 0.96 | 124 |
| (44) | | | | | |
| CRD-664 | DSG3-propeptide, EC3, EC4-CH2-CH3 | 113.9 | 6.4 | 1.14 | 98 |
| (45) | | | | | |
| CRD-924 | DSG3-propeptide, EC1 (C35V, C104V), EC2 | 157.4 | 5.6 | 1.80 | 218 |
| (46) | (C228A, C230V)-CH2-CH3 (LALA) | | | | |

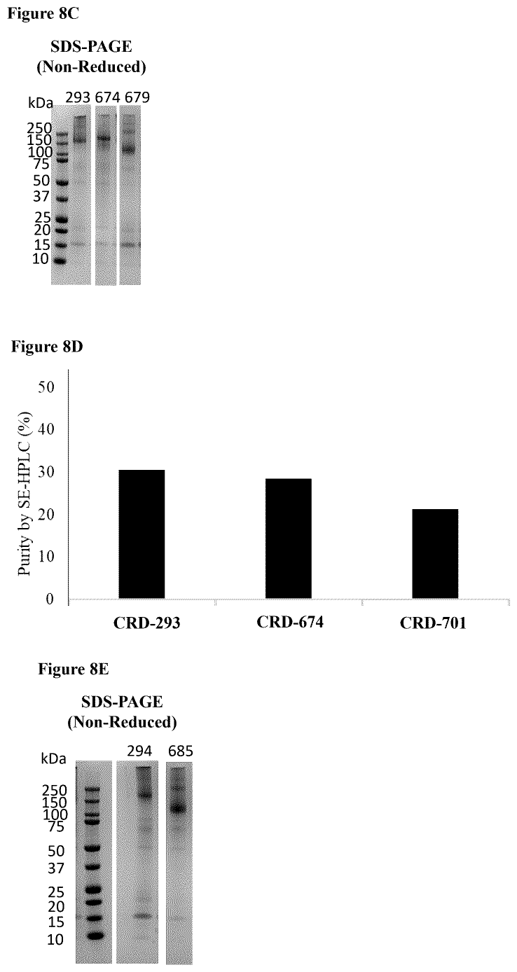

Molecule CRD-291 which contains the WT DSG3 extracellular domain was run on a non-reducing gel. A band was clearly present at about 160 kDa the expected weight of a dimer of the molecule (FIG. 8A). However, a smear was also observed at higher molecular weights, with a large amount of protein so highly aggregated that it could barely enter the gel. In contrast, molecule CRD-657 which contains mutations abolishing 4 cysteines (the two cysteines in EC1 and the two cysteines in EC2) showed greatly reduced aggregation, with the primary band observed at the expected molecular weight. Surprisingly, this benefit was only observed when the cysteines were mutated to valine or alanine. However, when the cysteines were converted to serine (CRD-700) no improvement in expression was observed. When the purity of monomers received was calculated, CRD-291 and CRD-657 were comparable, with each producing ~20% monomers (FIG. 8B). In contrast, CRD-700 produced essentially no monomers. Molecule CRD-662, which is a truncation containing only extracellular domains 1 and 2 also showed a marked reduction in aggregates, with the primary band at the expected weight (110 kDa for the dimer). Similar results were observed for the DSG1 containing molecules CRD-293 (WT), CRD-674 (5 cysteines mutated, the 3 in EC1 and the 2 in EC2) and CRD-679 (truncation) (FIG. 8C). Once again mutation of the cysteines to serine (CRD-701) produced no improvement in expression and reduced the yield of monomers (FIG. 8D). These results were also confirmed by SEC-HPLC (data not shown). Mutation of the cysteines within the truncated molecules produced even better yields (CRD-923 and CRD-924 in Tables 3 and 4).

Indeed, as can be seen in Tables 3 and 4, several, but not all, of the mutations/truncations produced more robust yields. For DSG1 the W28A mutation nearly doubled the yield produced, while the D53T and E117S mutations did not. When the three mutations were combined, the effect of the W28A mutation was lost. However, combination of the W28A mutation with a A106I mutation produced an even better effect on yield, which was matched only by a K43A, R44A double mutation. Several other mutation combinations also produced more modest improvements. The quintuple cysteine mutation within the EC1-EC2 fragment surprisingly produced one of the best yields.

For DSG3, the W28A mutation has no effect on yield, but the E117S mutation increased yield by more than 4 times. The W28A, D53T, E117D triple mutation and the W28A, A106I double mutations both produced a more than 3 times increase, but the K43A, R44A double mutation was by far the best, producing a 7X increase in yield. Truncations of DSG3 also produced improved yields. Removed of the 4th extracellular domain (EC4) produced a moderate improvement, expressing just EC1+EC2 or just EC3+EC4 produced even better yields. A truncation where only EC1 is removed gave an even better yield. Interestingly, truncations of DSG1 expressed alone did not improve yields and in fact produced worse yields than even the whole unmodified extracellular domain. For DSG3 the quadruple cysteine mutation within the EC1-EC2 fragment also produced a surprisingly high yield; indeed, it produced the greatest yield of any DSG3 protein tested in this batch.

Combinations that incorporated both DSG1 and DSG3 with several of the mutants and truncations were examined. The molecule containing both the WT DSG1 extracellular domain and the WT DSG3 extracellular domain (CRD-294) produced a smear at high molecular weight on a non-reducing gel (FIG. 8E). Use of truncated forms of both DSG1 and DSG3 (just the EC1 and EC2 domains of each; CRD-685) served to reduce the formation of aggregates and produced a primary band at the expected molecular weight. These results were also confirmed by SEC-HPLC (data not shown). The full results of molecule production are summarized in Table 5. Inclusion of the W28A mutation and removal of the EC4 domain both produced modest improvements in yield, but inclusion of only the EC1 and EC2 was greatly superior and when this was combined with the W28A mutation a greater than 4×increase in yield was observed. Once again, the EC1-EC2 truncations with all of the cysteines mutated produced far and away the best yields. This was true in both molecules that combined the DSG1 EC1-EC2 truncation with the DSG3 EC1-EC2 truncation (CRD-925, CRD-926). Several other tested mutations did not produce improved results.

TABLE 5
Batch III of DSG1 and DSG3 combination molecules of the invention

| Identifier | | | | | |
|---|---|---|---|---|---|
| SEQ ID | | MW | | | Yield |
| NO: ( ) | Molecules Description | (kDa) | pI | EC | (mg/L) |
| CRD-294 | DSG1-propeptide, EC1, EC2, EC3, EC4- | 160.7 | 5.6 | 0.98 | 31 |
| (9 + 10) | CH2-CH3 hole | | | | |
| | DSG3-propeptide, EC1, EC2, EC3, EC4- | | | | |
| | CH2-CH3 knob | | | | |
| CRD-683 | DSG1-propeptide, EC1 (W28A), EC2, EC3, | 161.8 | 5.7 | 0.91 | 56 |
| (47 + 48) | EC4-CH2-CH3 hole | | | | |
| | DSG3-propeptide, EC1 (W28A), EC2, EC3, | | | | |
| | EC4-CH2-CH3 knob | | | | |
| CRD-684 | DSG1-propeptide, EC1, EC2, EC3-CH2-CH3 | 137.0 | 5.6 | 1.05 | 64 |
| (49 + 50) | hole | | | | |
| | DSG3-propeptide, EC1, EC2, EC3-CH2-CH3 | | | | |
| | knob | | | | |
| CRD-685 | DSG1-propeptide, EC1, EC2-CH2-CH3 hole | 110.7 | 6.0 | 0.99 | 91 |
| (51 + 52) | DSG3-propeptide, EC1, EC2-CH2-CH3 knob | | | | |
| CRD-686 | DSG1-propeptide, EC1 (W28A), EC2-CH2- | 110.4 | 6.0 | 0.89 | 126 |
| (53 + 54) | CH3 hole | | | | |
| | DSG3-propeptide, EC1 (W28A), EC2-CH2- | | | | |
| | CH3 knob | | | | |
| CRD-702 | DSG3-pro-peptide, EC1 (N87Q, N157Q), | 162.4 | 5.6 | 0.92 | 12 |
| (57) | EC2, EC3, EC4-CH2-CH3 | | | | |
| CRD-703 | DSG1-pro-peptide, EC1 (N87Q, N157Q), | 161.8 | 5.7 | 0.98 | 39 |
| (58) | EC2, EC3, EC4-CH2-CH3 | | | | |
| CRD-925 | DSG1-propeptide, EC1 (C35V, C54A, | 158.2 | 5.69 | 1.80 | 122 |
| (59 + 60) | C104V), EC2 (C230A, C232V)-CH2-CH3 | | | | |
| | LALA hole | | | | |
| | DSG3-propeptide, EC1 (C35V, C104V), EC2 | | | | |
| | (C228A, C230V)-CH2-CH3 LALA knob | | | | |
| CRD-926 | DSG1-propeptide, EC1 (C35V, C54A, | 157.3 | 5.61 | 1.80 | 194 |
| (61 + 62) | C104V), EC2 (C230A, C232V)-CH2-CH3 | | | | |
| | LALA knob | | | | |
| | DSG3-propeptide, EC1 (C35V, C104V), EC2 | | | | |
| | (C228A, C230V)-CH2-CH3 LALA hole | | | | |

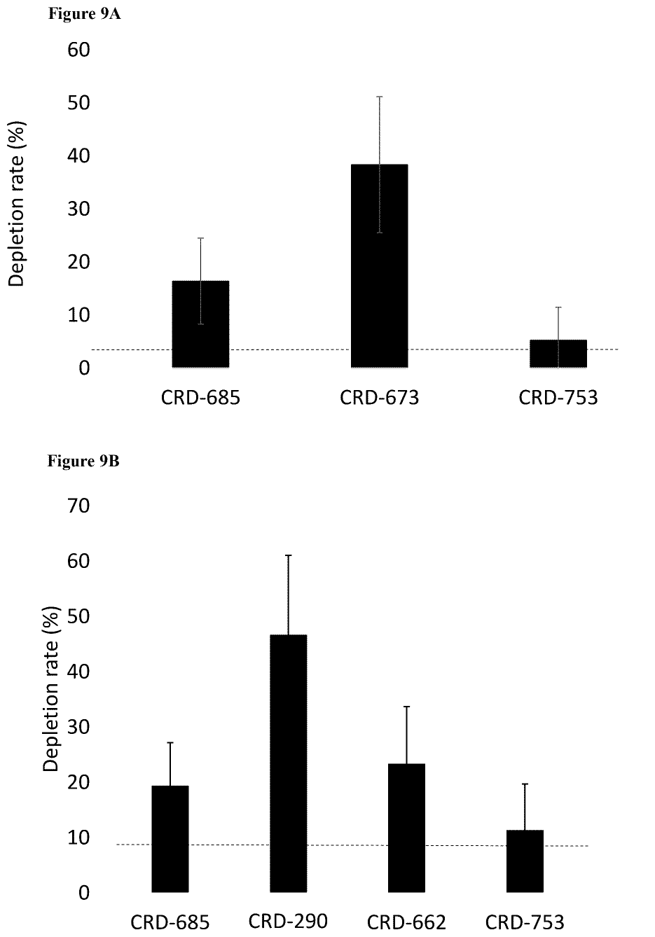

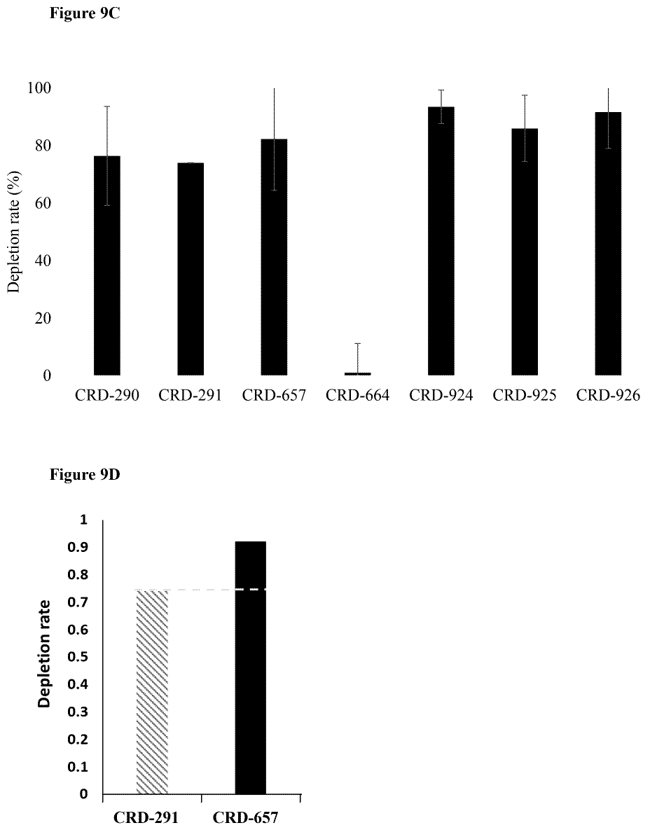

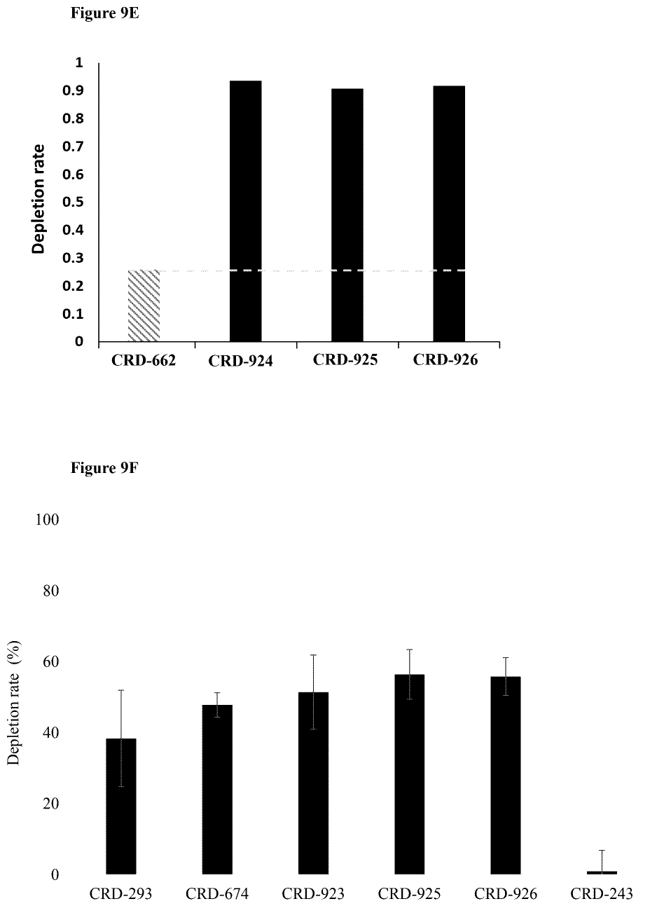

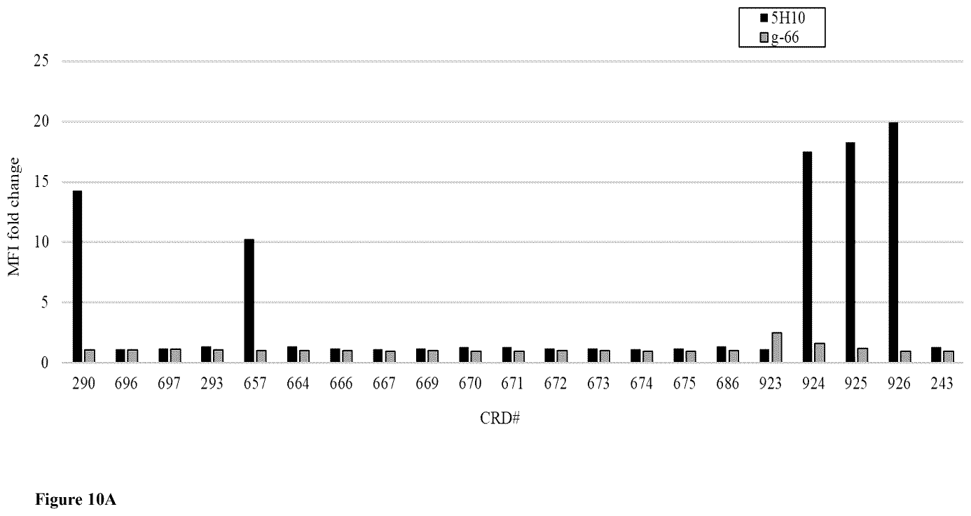

Autoantibody depletion using the new molecules was tested as before. 5 human sera samples positive for anti-DSG1 IgG were depleted using a DSG1/DSG3 knob-in-hole combination molecule (CRD-685) and a less aggregate/higher yield DSG1 only molecule (CRD-673). Both molecules produced greater depletion than an Fc-Fusion molecule with an irrelevant extracellular domain (CRD-753), and in particular the DSG1 only molecule produced greater than 40% depletion (FIG. 9A). Similarly, 6 human sera samples positive for anti-DSG3 IgG were depleted using a DSG1 EC 1/2 fragment and DSG3 EC 1/2 fragment combined by the knob-in-hole approach (CRD-685), a DSG3 EC 1/2 fragment Fc Fusion (CRD-662) and DSG-3 ECD EC1-EC4 (CRD-290) molecules. Although both CRD-685 and 662 were able to produce modest depletion, CRD-290 depleted roughly 50% of the antibodies (FIG. 9B).

Next 8 human sera samples positive for anti-DSG3 IgG were depleted using the DSG3 molecule with the full extracellular domain and no dimerization domain (CRD-290), the DSG3 molecule with the full extracellular domain and CH2 and CH3 (CRD-291), the DSG3 molecule with the full extracellular domain and all cysteines in EC1 and EC2 mutated and CH2-CH3 (CRD-657), the DSG3 molecule containing only EC3 and EC4 and CH2-CH3 (CRD-664), the DSG3 molecule with only EC1 and EC2 and all cysteines in EC1 and EC2 mutated and CH2-CH3 (CRD-924), and two DSG3/DSG1 knob in hole combined molecules with only fragments of each protein containing the EC1 and EC2 domains used and where all cysteines are mutated (CRD-925, CRD-926). All molecules except the EC3/EC4 only fragments produced robust depletion of greater than 75% (FIG. 9C). Surprisingly, a molecule with all cysteines of EC1 and EC2 mutated to valine or alanine was as good if not better than the molecules with the wild-type extracellular domain (CRD-657 vs CRD-290/1, FIG. 9C-D). Even more surprising the use of just the EC1/EC2 fragment with the cysteines mutated was even better than the full-length molecule with mutated cysteines (CRD-924 vs CRD-657) and the truncated EC1/EC2 fragment without mutations (CRD-924 vs CRD662) (FIG. 9E). Inclusion of the DSG1 EC1/EC2 fragment with mutated cysteines (CRD-925 and CRD-926) did not significantly impact binding and indeed greater than 80% depletion of anti-DSG3 was still achieved with this molecule.

Similarly, 3 human sera samples positive for anti-DSG1 IgG were depleted using the DSG1 molecule with the full extracellular domain and CH2 and CH3 (CRD-293), the DSG1 molecule with the full extracellular domain and all cysteines in EC1 and EC2 mutated and CH2-CH3 (CRD-674), the DSG1 molecule with only EC1 and EC2 and all cysteines in EC1 and EC2 mutated and CH2-CH3 (CRD-923), and the two DSG3/DSG1 knob in hole combined molecules with only fragments of each protein containing the EC1 and EC2 domains used and where all cysteines are mutated (CRD-925, CRD-926). An Fc fusion to an irrelevant extracellular domain (CRD-243) was used as a negative control. All DSG1 containing molecules produced depletion of about 40% or greater (FIG. 9F). Surprisingly, the molecule with all cysteines of EC1 and EC2 mutated was as better than the molecule with the wild-type extracellular domain (CRD-674 vs CRD-293). Even more surprising the use of just the EC1/EC2 fragment with the cysteines mutated was comparable to or even better than the full-length molecule with mutated cysteines (CRD-923 vs CRD-674). Even more surprising the inclusion of the DSG3 EC1/EC2 fragment with mutated cysteines not only did not reduce binding it actually increased it slightly producing very nearly a 60% depletion.

### Example 4

Having examined protein complexes and single chains that include a dimerization domain, soluble molecules of just the extracellular domains of DSG1/3 or fragments or mutants thereof were also examined. The resulting yields from producing these proteins are summarized in Table 6. All of these polypeptides produced high yields.

TABLE 6
DSG1 and DSG3 molecules without a dimerization domain of the invention.

| Identifier | | | | | |
|---|---|---|---|---|---|
| SEQ ID | | MW | | | Yield |
| NO: ( ) | Molecules Description | (kDa) | pI | EC | (mg/L) |
| CRD-290 | DSG3 ECD-propeptide, EC1, EC2, EC3, | 58.2 | 5.5 | 0.77 | 100 |
| (6) | EC4-His Avi tag | | | | |
| CRD-688 | DSG3 ECD-propeptide, EC1 (W28A), EC2, | 58.1 | 5.5 | 0.67 | 138 |
| (63) | EC3, EC4-His Avi tag | | | | |
| CRD-689 | DSG3 ECD-propeptide, EC1, EC2-His Avi | 32.1 | 5.6 | 0.54 | 104 |
| (64) | tag | | | | |
| CRD-690 | DSG3 ECD-propeptide, EC1 (W28A), EC2- | 32 | 5.6 | 0.37 | 172 |
| (65) | His Avi tag | | | | |
| CRD-691 | DSG3 ECD-propeptide, EC1 (C35V, C104V), | 58.1 | 5.5 | 0.76 | 114 |
| (66) | EC2 (C228A, C230V), EC3, EC4-His Avi tag | | | | |
| CRD-692 | DSG3 ECD-propeptide, EC1 (E95G, D129G), | 58 | 5.7 | 0.77 | 72 |
| (67) | EC2 (D162G), EC3, EC4-His Avi tag | | | | |
| CRD-693 | DSG3 ECD-propeptide, EC1, EC2 (E205G, | 58 | 5.7 | 0.77 | 72 |
| (68) | D239G), EC3 (D271G), EC4-His Avi tag | | | | |
| CRD-292 | DSG1 ECD-propeptide, EC1, EC2, EC3, | 57.9 | 5.6 | 0.84 | 92 |
| (5) | EC4-His Avi tag | | | | |
| CRD-695 | DSG1 ECD-propeptide, EC1 (W28A), EC2, | 57.8 | 5.6 | 0.75 | 104 |
| (69) | EC3, EC4-His Avi tag | | | | |
| CRD-696 | DSG1 ECD-propeptide, EC1, EC2-His Avi | 32.7 | 5.9 | 0.83 | 80 |
| (70) | tag | | | | |
| CRD-697 | DSG1 ECD-propeptide, EC1 (W28A), EC2- | 32.5 | 5.9 | 0.67 | 108 |
| (71) | His Avi tag | | | | |
| CRD-698 | DSG1 ECD-propeptide, EC1 (C35V, C54A, | 57.8 | 5.6 | 0.84 | 90 |
| (72) | C104V), EC2 (C230A, C232V), EC3, EC4- | | | | |
| | His Avi tag | | | | |
| CRD-699 | DSG1 ECD-propeptide, EC1 (E95G, D129G), | 57.7 | 5.8 | 0.84 | 86 |
| (73) | EC2 (D162G), EC3, EC4-His Avi tag | | | | |

### Example 5

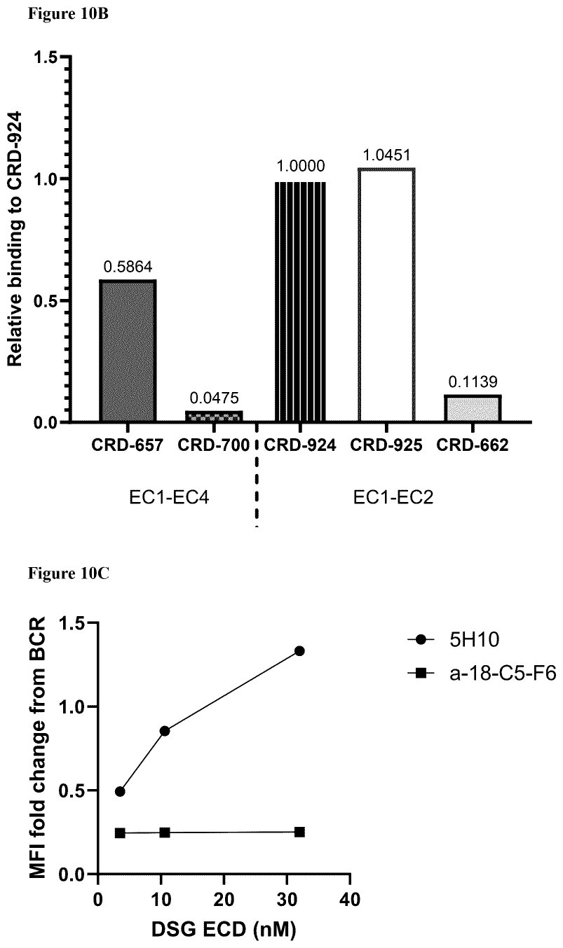

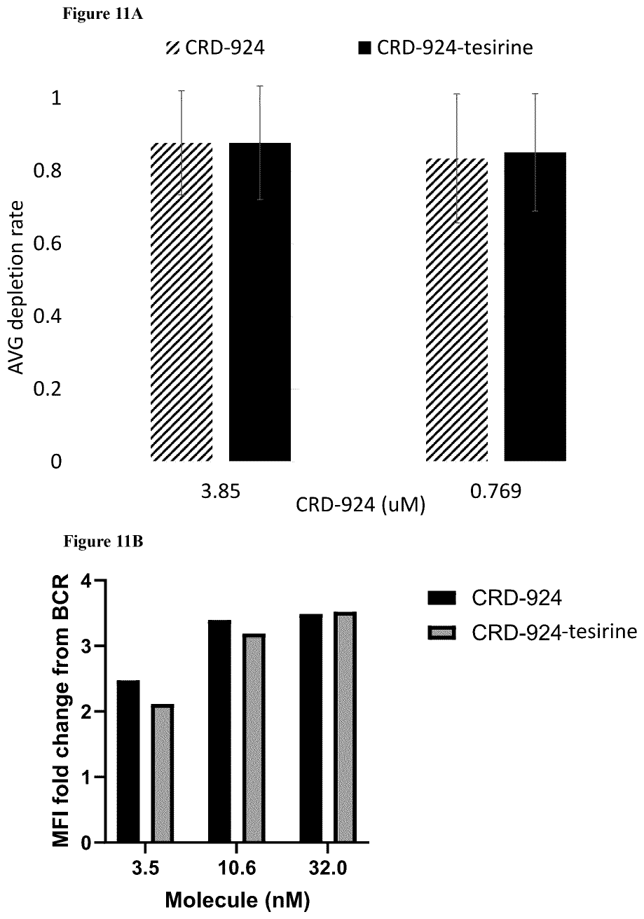

In order to test the ability of the molecules to target autoreactive B cells themselves and not merely sequester autoantibodies, a murine hybridoma expressing BCR against DSG-3 was purchased (5H10 hybridoma, University of Toledo). A rat hybridoma expressing BCR against an irrelevant protein (g-66 hybridoma) was used as a negative control. APC labeled CRD-290 was incubated with the hybridoma cells for 40 min at 4 degrees Celsius. Following incubation, cells were washed with FACS buffer (DPBS with 1% FBS) twice and analyzed on a flow cytometry (CytoFlex by Beckman Coulter). Cells incubated with APC alone were used as a negative control. Mean fluorescent intensity (MFI) fold change from background values were computed by dividing APC-MFI of CRD-290 stained hybridoma cells by its' negative control background MFI. CRD-290 strongly bound to the hybridoma expressing BCR against DSG3, whereas only minimal, non-specific, binding to the control hybridomas was observed (FIG. 10A). When a molecule with an irrelevant extracellular domain (CRD-243) was used, no binding to the hybridoma expressing BCR against DSG3 was observed. DSG1 containing molecules CRD-696, CRD-697, CRD-293, CRD-666, CRD-667, CRD-669, CRD-670, CRD-671, CRD-672, CRD-673, CRD-674, CRD-675 and CRD-923 all showed no binding and were comparable to the negative control. Interestingly, CRD-664 which contains a fragment of EC3-EC4 of DSG3 also showed no binding and was comparable to the negative control. This suggests that, for this hybridoma at least only, anti-EC1 and EC2 BCR is produced. CRD-657 showed strong specific binding to the DSG3 hybridoma indicating that abolishing the cysteines in EC1 and EC2 did not greatly diminish BCR binding. However, when those cysteines were converted to serines, hybridoma binding was greatly reduced (FIG. 10B). Specific binding was also observed when the molecule did not contain the CH2 and CH3 domains (CRD-691; FIG. 10C). Surprisingly however, CRD-686 which is a heterodimer of the EC1-EC2 fragment of DSG3 and the EC1-EC2 fragment of DSG1 each containing a W28A mutation did not produce BCR binding and was also comparable to the negative control. The strongest hybridoma binding however, was produced by the molecules comprising only the EC1-EC2 fragment of DSG3 with all 4 cysteines mutated (CRD-924, CRD-925 and CRD-926). While the homodimer with two DSG3 fragments was superior to the full length (wild-type or mutant; CRD-924 vs CRD290 and CRD-657, respectively) it was greatly surprising that the heterodimers that also included the EC1-EC2 fragment of DSG1 with all 5 cysteines mutated were even better at specific hybridoma binding (CRD-925/6 vs. CRD-924). The mutated EC1-EC2 fragments were also superior as compared to the wild-type EC1-EC2 fragment (FIG. 10B). This data indicates that the molecules of the invention can be used to target B cells and provide a lasting cure to PV/PF and not just transiently reduce autoantibody levels. It further shows that a fragment with just EC1 and EC2 is sufficient for targeting B cells and that not only is mutating the cysteines of this fragment not detrimental, but it actually enhances specific targeting to B-cells. Lastly, an importantly for the development of a therapeutic that can be used against both DSG1 and DSG3 autoantibody related diseases, the heterodimer containing both the DSG1 and DSG3 EC1-EC2 fragments with all cysteines mutated were the most effect molecules for specific hybridoma binding.

As CRD-924 was such a promising molecule, it was tested against a wide variety of hybridomas to confirm its specificity only for DSG3. The following hybridomas were tested for binding: 5H10 (DSG3), g-66 (ACHRG), g-50-H1-E2 (ACHRG), g-22-F5-C1 (ACHRG), G-63 (ACHRG), g-86-F6-G9 (ACHRG), g-51-G4-H7 (ACHRG), B-73 (ACHRB), A-CYT-155 (ACHR cytoplasmatic region), b-CYT-148 (ACHR cytoplasmatic region), TSHR-51 (TSHR), LK-4 (Integrin), AP-3 (Integrin), 2HX2 (adenovirus group specific antigen), 204-4 (Coxsackie virus B4), 4E8C12 (E. coli), 72A1 (Epstein-Barr virus), H25B10 (Hepatitis B virus), H16-L10-4R5 (Influenza virus), 2D12 (Yellow fever virus), C273 (CD20), CKMM14.52 (CK-MM), A-18-C5-F6 (ACHRA), A-192 (ACHRA), a-122-H1-G6 (ACHRA), A-198 (ACHRA), a-55-E5-G3 (ACHRA), a-6-1-H2-F6 (ACHRA) and TIB175 (ACHRA). Appreciable binding was detected only to hybridoma 5H10, indicating that CRD-924 is highly specific to anti-DSG3 B cells.

### Example 6: Specific B Cell Killing by Tesirine Conjugate

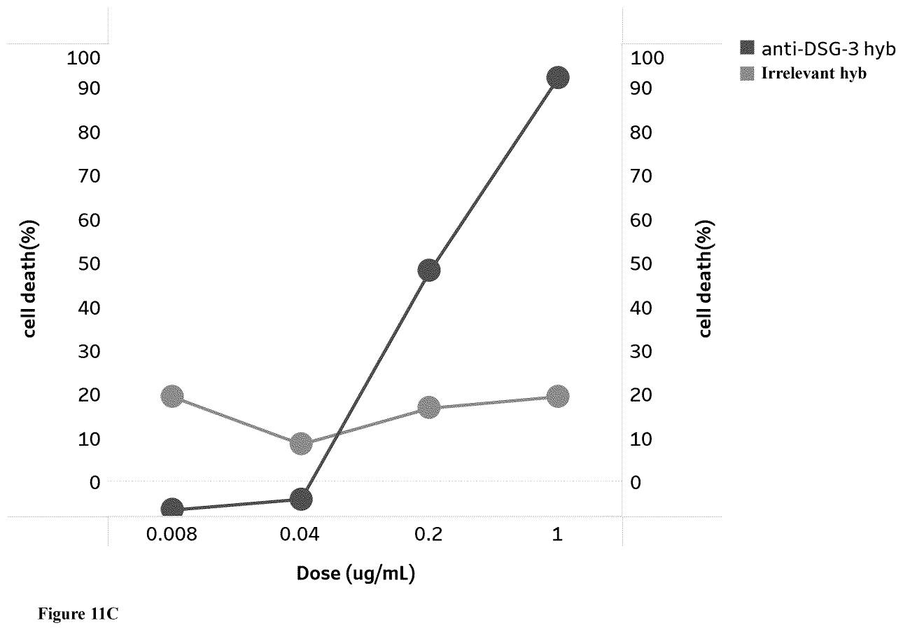

CRD-924 was also tested for is ability to kill Anti-DSG-3 B cell hybridoma cells. CRD-924 was conjugated to the pyrrolobenzodiazepine (PBD) chemotherapeutic Tesirine, by using Fc-native cysteine conjugation. Unconjugated CRD-924 was used as a control. Eight human sera samples positive for anti-DSG3 IgGs were used to test depletion by the unconjugated and conjugated molecule at two different concentrations (3.85 μM or 0.769 uM). The addition of Tesirine did not impact CRD-924's ability to deplete anti-DSG3 IgG (FIG. 11A). Next, binding to and killing of 5H10 hybridoma cells was measured. Conjugation of Tesirine did not impact CRD-924's ability to bind to the anti-DSG3 hybridoma (FIG. 11B). Death rate was calculated as the percent increase produced by the tesirine conjugate over the unconjugated CRD-924. The CRD-924-tesirine conjugate produced greatly superior cell killing as compared to the unconjugated molecule (>90% increase) and superiority was seen even at a concentration of only 0.2 μg/ml (FIG. 11C). Further, this killing was specific as no killing of an irrelevant non-DSG hybridoma in the coculture was observed. Thus, tesirine is a highly potent effector moiety whose conjugation can be used to increase the B cell killing capacity of Ig-like molecules of the invention beyond the insufficient killing produced merely by an unmodified Fc. It will be understood that tesirine conjugation to any of the molecules of the invention will have the same effect and boost B cell killing.

Other effector molecules are tested. These include anti-DSG-1/3 molecules of the invention conjugated to alpha-amanitin, Tesirine, Dxd, PNU-159682, MMAE, MMAF, and triptolide. All show superior killing to that produced by an unmodified Fc domain. Fc domains with mutations that increase ADCC are also tested. Fc mutations such as are described hereinabove are generated in the Fc and killing is tested in anti-DSG-1 and anti-DSG-3 hybridomas. Killing is specific to these hybridomas and not hybridomas against other targets and the killing is superior to that produced by an unmodified Fc.

It has been well established that auto-reactive B cells can be found in naïve/healthy mice, especially inbred strains (see for example Ding and Yan, “Regulation of autoreactive B cells: checkpoints and activation”, Arch. Immunol. Ther. Exp., 2007, 55, 83-89; Wang et al., “The naïve B cell repertoire predisposes to antigen-induced systemic lupus erythematosus” J Immunol. 2003 May 1;170 (9): 4826-32; and Fereidan-Esfahani et al., “IgM natural autoantibodies in physiology and the treatment of disease”, Methods Mol Biol. 2019:1904:53-81). To confirm this, blood is drawn from naïve 6-8-week-old C57B16 inbred female mice and a magnetic bead-based immunoassay is performed to measure anti-DSG-3 or anti-DSG1 antibody titers. All mice are found to be positive for antibodies. The presence of these autoreactive antibodies indicates that autoreactive B cells are present even before immunization with DSG1/DSG3 fragments.

To test the ability of the molecules of the invention to kill these autoreactive B cells, female C57BL6 mice at the age of 6-8 weeks are immunized intravenously with naked DSG3-Fc molecules of the invention or with drug conjugated DSG3-Fc molecules of the invention (0.5 mg/kg or higher dose) twice weekly for a total 6-8 injections. Subcutaneous injection is also tested. Negative control groups include mice that receive PBS and mice that are administered an irrelevant Ig-like molecule conjugated to the drug. Serum samples are isolated during the experiment and autoantibody titer is evaluated. At the end of the immunization period relative antibody titers to DSG3 are compared using DSG3-specific-immunoassay (ELISA). Following the sixth or eighth immunization, animals immunized with naked DSG3-Fc exhibit an increase in anti-DSG3 titer, while no titer elevation is observed in animals immunized with drug-conjugated DSG3-Fc. This demonstrates that the drug conjugate molecules of the invention kill PV and PF autoreactive B cells and can treat these diseases. The same tests are also run with DSG-1 molecules of the invention and combination DSG3-DSG1 molecules of the invention.

PV/PF is also induced in mice by the subcutaneous injection of DSG-1/DSG-3 ECD fragments. The ability to treat PV/PF in this organism with the molecules of the invention is confirmed. Serum is taken and antibody titer levels are monitored. Not only do the molecules of the invention kill target B cells, but they also reduce circulating antibody levels.

The various molecules of the invention are tested in vivo. These molecules are all found to effectively treat PV and PF, kill autoreactive B cells and reduce autoantibody titer levels in vivo. All tested effector moieties are found superior to Fc.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A polypeptide comprising a fragment of an extracellular domain of Desmoglein 1 (DSG1) or Desmoglein 3 (DSG3) and at least one mutation that increases solubility of said fragment, decreases aggregation of said fragment or both.

2. (canceled)

3. The polypeptide of claim 1, wherein said DSG1 extracellular domain comprises SEQ ID NO: 2 and said mutation is selected from: C9V, C28A, C78V, C204A, C206V, W2A, D27T, E89S, A80I, K17A, R18A, R10A, R97A, C9S, C28S, C78S, C204S, C206S, C426S, E69G, D103G, D136G, E179G, D215G, D247G, E231G, E291G, E332G, N61Q, and N131Q or said DSG3 extracellular domain comprises SEQ ID NO: 4 and said mutation is selected from: C9V, C78V, C202A, C204V, W2A, D27T, E89S, A80I, K17A, R18A, R10A, K97A, C9S, C78S, C202S, C204S, E69G, D103G, D136G, E179G, D213G, D245G, E229G, E289G, E330G, N61Q, and N131Q.

4. The polypeptide of claim 3, wherein said at least one mutation SEQ ID NO: 2 is:
a. C9V, C28A, C78V, C204A and C206V;
b. W2A;
c. D27T;
d. E89S;
e. W2A, D27T and E89S;
f. W2A and A80I;
g. K17A and R18A;
h. R10A, K17A, R18A, and R97A;
i. C9V, C28A, C78V, C204A, C206V and C426S;
j. E69G, D103G, and D136G;
k. E179G, D215G and D247G;
1. E231G, E291G, and E332G;
m. N61Q and N131Q; or
n. C9S, C28S, C78S, C204S, and C206S.

5. (canceled)

6. The polypeptide of claim 3, wherein said at least one mutation in SEQ ID NO: 4 is:
a. C9V, C78V, C202A and C204V;
b. W2A;
c. E89S;
d. W2A, D27T and E89S;
e. W2A and A80I;
f. K17A and R18A;
g. R10A, K17A, R18A, and K97A;
h. E69G, D103G, and D136G;
i. E179G, D213G and D245G;
j. E229G, E289G, and E330G;
k. N61Q and N131Q; or
l. C9S, C78S, C202S, and C204S.

7. The polypeptide of claim 3, wherein said fragment consists of extracellular domains (ECs) 1 and 2 of DSG1 or DSG3 and said polypeptide is devoid of extracellular domains (ECs) 3 and 4 of DSG1 or DSG3 and wherein EC1 of DSG1 consists of SEQ ID NO: 114, EC2 of DSG1 consists of SEQ ID NO: 115, EC3 of DSG1 consists of SEQ ID NO: 116, EC4 of DSG1 consists of SEQ ID NO: 117, EC1 of DSG3 consists of SEQ ID NO: 118, EC2 of DSG3 consists of SEQ ID NO: 119, EC3 of DSG3 consists of SEQ ID NO: 120 and EC4 of DSG3 consists of SEQ ID NO: 121.

8. The polypeptide of claim 7, wherein said fragment comprises or consists of an amino acid sequence selected from SEQ ID NO: 74-85, 90-103 and 108-113, optionally wherein said fragment comprises or consists of an amino acid sequence selected from SEQ ID NO: 81, 82, 90, 100 and 108.

9. (canceled)

10. (canceled)

11. The polypeptide of claim 1, further comprising an effector moiety, wherein said effector moiety is selected from an Fc domain comprising at least one mutation that increases ADCC or CDC, an amatoxin/amanitin, an anthracycline, an anthramycin-based dimer, a calicheamicin, camptothecin or an analog thereof, a duocarmycin, triptolide and a tubulin inhibitor, optionally wherein said effector moiety is conjugated to said polypeptide by a linker.

12. (canceled)

13. (canceled)

14. (canceled)

15. (canceled)

16. The polypeptide of claim 11, wherein said effector moiety is selected from: alpha-amanitin, PNU-159682, tesirine, deruxtecan (Dxd), mertansine, monomethyl auristatin E (MMAE), monomethyl auristatin F (MMAF) and a combination thereof.

17-63. (canceled)

64. A pharmaceutical composition comprising a polypeptide of claim 1 and a pharmaceutically acceptable carrier, excipient or adjuvant.

65. A method of treating Pemphigus vulgaris or Pemphigus foliaceus in a subject in need thereof, the method comprising administering to said subject a pharmaceutical composition of claim 64, thereby treating Pemphigus vulgaris or Pemphigus foliaceus.

66. (canceled)

67. (canceled)

68. (canceled)

69. The method of claim 65, wherein said pharmaceutical composition comprises a polypeptide of claim 16.

70. (canceled)

71. The method of 65, wherein said treating comprises decreasing the concentration of circulating autoantibodies against DSG1, DSG3 or both.

72. The method of claim 65, wherein said composition comprises an effector moiety and said treating comprises killing autoreactive B cells producing anti-DSG1 or DSG3 autoantibodies.

73. (canceled)

74. A nucleic acid molecule encoding a polypeptide of claim 1.

75. (canceled)

76. (canceled)

77. (canceled)

78. A method for producing a protein the method comprising:
obtaining a first fragment of an extracellular domain of DSG1 or an analog or derivative thereof or a fragment of an extracellular domain of DSG3 or an analog or derivative thereof, and a second fragment of an extracellular domain of DSG1 or an analog or derivative thereof or a fragment of an extracellular domain of DSG3 or an analog or derivative thereof, linking said first fragment to a first dimerization domain to produce a first polypeptide chain and linking said second fragment to a second dimerization domain to produce a second polypeptide chain wherein said first and second dimerization domains are capable of dimerizing with each other, and contacting said first polypeptide and said second polypeptide under conditions sufficient to induce said dimerization; or
culturing a host cell comprising one or more vectors comprising a nucleic acid sequence encoding at least two polypeptide chains, wherein the two polypeptide chains are produced by:
i. obtaining a first fragment of an extracellular domain of DSG1 or an analog or derivative thereof or a fragment of an extracellular domain of DSG3 or an analog or derivative thereof and a second fragment of an extracellular domain of DSG1 or an analog or derivative thereof or a fragment of an extracellular domain of DSG3 or an analog or derivative thereof; and
ii. linking said first fragment to a first dimerization domain to produce a first polypeptide chain and linking said second fragment to a second dimerization domain to produce a second polypeptide chain wherein said first and second dimerization domains are capable of dimerizing with each other;
thereby producing a protein.

79. (canceled)

80. (canceled)

81. (canceled)

82. The method of claim 78, further comprising producing at least one mutation in a cadherin domain of the protein or truncating the protein to remove at least one cadherin domain or a portion thereof, measuring aggregation of the mutated or truncated protein and selecting a mutated or truncated protein with reduced aggregation.

83. (canceled)

84. A method for producing a protein the method comprising:
a. obtaining a fragment of an extracellular domain of DSG1 or an analog or derivative thereof or a fragment of an extracellular domain of DSG3 or an analog or derivative thereof;
b. generating at least one mutation in said fragment to produce a mutated fragment;
c. measuring solubility, aggregation or both of the mutated fragment; and
d. selecting at least one mutated fragment with increased solubility, decreased aggregation or both as compared to the obtained fragment;
thereby producing a protein.

85. The method of claim 78, further comprising linking an effector moiety to at least one of said polypeptide chains or said mutated fragment, wherein said effector moiety is selected from an Fc domain comprising at least one mutation that increases ADCC or CDC, an amatoxin/amanitin, an anthracycline, an anthramycin-based dimer, calicheamicin, camptothecin or an analog thereof, a duocarmycin, triptolide and a tubulin inhibitor.

86. (canceled)

87. The method of claim 85, wherein said effector moiety is selected from: alpha-amanitin, PNU-159682, tesirine, deruxtecan (Dxd), mertansine, monomethyl auristatin E (MMAE), monomethyl auristatin F (MMAF) and a combination thereof.

88. (canceled)

89. (canceled)

90. (canceled)

91. A method of determining suitability of a subject in need thereof, the method comprising receiving a sample from the subject, contacting said sample with a protein comprising a fragment of an extracellular domain of Desmoglein 1 (DSG1) or Desmoglein 3 (DSG3) and at least one mutation that increases solubility of said fragment, decreases aggregation of said fragment or both determining binding of autoantibodies within said sample to said protein or said composition, wherein binding of autoantibodies to said protein or said composition indicates said subject is suitable to be treated by a method of claim 65, thereby determining suitability of the subject to be treated.


SEQUENCE LISTING (171 sequences)

SEQ ID NO: 1 - protein, 474 aa, Homo sapiens
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 2 - protein, 448 aa, Homo sapiens
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 3 - protein, 476 aa, Homo sapiens
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 4 - protein, 450 aa, Homo sapiens
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 5 - protein, 474 aa, synthetic construct; identical to SEQ ID NO: 1
  REGION 1..474: note = Synthetic

SEQ ID NO: 6 - protein, 476 aa, synthetic construct; identical to SEQ ID NO: 3
  REGION 1..476: note = Synthetic

SEQ ID NO: 7 - protein, 721 aa, synthetic construct
  REGION 1..721: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 723 aa, synthetic construct
  REGION 1..723: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 721 aa, synthetic construct
  REGION 1..721: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 10 - protein, 723 aa, synthetic construct
  REGION 1..723: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 593 aa, synthetic construct
  REGION 1..593: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 821 aa, synthetic construct
  REGION 1..821: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 595 aa, synthetic construct
  REGION 1..595: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 819 aa, synthetic construct
  REGION 1..819: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 726 aa, synthetic construct
  REGION 1..726: note = Synthetic
EFRIQVRDYNTKNGTIKWHSIRRQKREAIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 16 - protein, 726 aa, synthetic construct
  REGION 1..726: note = Synthetic
EFRIQVRDYNTKNGTIKWHSIRRQKREWIKFAAACREGEDNSKRNPIAKIHSTCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 17 - protein, 726 aa, synthetic construct
  REGION 1..726: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLSRPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 18 - protein, 726 aa, synthetic construct
  REGION 1..726: note = Synthetic
EFRIQVRDYNTKNGTIKWHSIRRQKREAIKFAAACREGEDNSKRNPIAKIHSTCAANQQVTYRISGVGIDQPPYGIFVINQKTGEINITSIVDREVTPFFIIYCRALNSMGQDLSRPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 19 - protein, 726 aa, synthetic construct
  REGION 1..726: note = Synthetic
EFRIQVRDYNTKNGTIKWHSIRRQKREAIKFAAACREGEDNSKRNPIAKIHSDCAANQQVTYRISGVGIDQPPYGIFVINQKTGEINITSIVDREVTPFFIIYCRILNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 20 - protein, 726 aa, synthetic construct
  REGION 1..726: note = Synthetic
EFRIQVRDYNTKNGTIKWHSIRRQKREWIKFAAACREGEDNSAANPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 21 - protein, 726 aa, synthetic construct
  REGION 1..726: note = Synthetic
EFRIQVRDYNTKNGTIKWHSIRRQKREWIKFAAACAEGEDNSAANPIAKIHSDCAANQQVTYRISGVGIDQPPYGIFVINQKTGEINITSIVDREVTPFFIIYCRALNSMGQDLERPLELRVAVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 22 - protein, 726 aa, synthetic construct
  REGION 1..726: note = Synthetic
EFRIQVRDYNTKNGTIKWHSIRRQKREWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTSTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 23 - protein, 726 aa, synthetic construct
  REGION 1..726: note = Synthetic
EFRIQVRDYNTKNGTIKWHSIRRQKREWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 24 - protein, 726 aa, synthetic construct
  REGION 1..726: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDRGVTPFFIIYCRALNSMGQDLERPLELRVRVLDINGNPPVFSMATFAGQIEENSNANTLVMILNATDAGEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 25 - protein, 726 aa, synthetic construct
  REGION 1..726: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDRGQYGQYALAVRGSDRDGGADGMSAECECNIKILDVNGNIPYMEQSSYTIEIQENTLNSNLLEIRVIDLGEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 26 - protein, 726 aa, synthetic construct
  REGION 1..726: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQGNTLNSNLLEIRVIDLDEEFSANWMAVIFFISGNEGNWFEIEMNERTNVGILKVVKPLDYGAMQSLQLSIGVRNKAEFHHSIMSQYKLKASAISVTVLNVIGGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 27 - protein, 614 aa, synthetic construct
  REGION 1..614: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 28 - protein, 499 aa, synthetic construct
  REGION 1..499: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 29 - protein, 617 aa, synthetic construct
  REGION 1..617: note = Synthetic
EFRIQVRDYNTKNGTIKWHSIRRQKRMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 30 - protein, 505 aa, synthetic construct
  REGION 1..505: note = Synthetic
EFRIQVRDYNTKNGTIKWHSIRRQKRQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 31 - protein, 499 aa, synthetic construct
  REGION 1..499: note = Synthetic
EFRIQVRDYNTKNGTIKWHSIRRQKREWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 32 - protein, 728 aa, synthetic construct
  REGION 1..728: note = Synthetic
ELRIETKGQYDEEEMTMQQAKRRQKREAVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 33 - protein, 728 aa, synthetic construct
  REGION 1..728: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVSKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 34 - protein, 728 aa, synthetic construct
  REGION 1..728: note = Synthetic
ELRIETKGQYDEEEMTMQQAKRRQKREAVKFAKPCREGEDNSKRNPIAKITSTYQATQKITYRISGVGIDQPPFGIFVVDKNTGDINITAIVDREETPSFLITCRALNAQGLDVSKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 35 - protein, 728 aa, synthetic construct
  REGION 1..728: note = Synthetic
ELRIETKGQYDEEEMTMQQAKRRQKREAVKFAKPCREGEDNSKRNPIAKITSDYQATQKITYRISGVGIDQPPFGIFVVDKNTGDINITAIVDREETPSFLITCRILNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 36 - protein, 728 aa, synthetic construct
  REGION 1..728: note = Synthetic
ELRIETKGQYDEEEMTMQQAKRRQKREWVKFAKPCREGEDNSAANPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 37 - protein, 728 aa, synthetic construct
  REGION 1..728: note = Synthetic
ELRIETKGQYDEEEMTMQQAKRRQKREWVKFAKPCAEGEDNSAANPIAKITSDYQATQKITYRISGVGIDQPPFGIFVVDKNTGDINITAIVDREETPSFLITCRALNAQGLDVEKPLILTVAILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 38 - protein, 728 aa, synthetic construct
  REGION 1..728: note = Synthetic
ELRIETKGQYDEEEMTMQQAKRRQKREWVKFAKPVREGEDNSKRNPIAKITSDYQATQKITYRISGVGIDQPPFGIFVVDKNTGDINITAIVDREETPSFLITVRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQAEVNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 39 - protein, 728 aa, synthetic construct
  REGION 1..728: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDRGETPSFLITCRALNAQGLDVEKPLILTVKILDINGNPPVFSQQIFMGEIEENSASNSLVMILNATDAGEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 40 - protein, 728 aa, synthetic construct
  REGION 1..728: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDRGQASSYRLVVSGADKDGEGLSTQCECNIKVKDVNGNFPMFRDSQYSARIEENILSSELLRFQVTDLGEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 41 - protein, 728 aa, synthetic construct
  REGION 1..728: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEGNILSSELLRFQVTDLDEEYTDNWLAVYFFTSGNEGNWFEIQTDPRTNEGILKVVKALDYGQLQSVKLSIAVKNKAEFHQSVISRYRVQSTPVTIQVINVRGGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 42 - protein, 612 aa, synthetic construct
  REGION 1..612: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 43 - protein, 497 aa, synthetic construct
  REGION 1..497: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 44 - protein, 619 aa, synthetic construct
  REGION 1..619: note = Synthetic
ELRIETKGQYDEEEMTMQQAKRRQKRQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 45 - protein, 509 aa, synthetic construct
  REGION 1..509: note = Synthetic
ELRIETKGQYDEEEMTMQQAKRRQKRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 46 - protein, 497 aa, synthetic construct
  REGION 1..497: note = Synthetic
ELRIETKGQYDEEEMTMQQAKRRQKREWVKFAKPVREGEDNSKRNPIAKITSDYQATQKITYRISGVGIDQPPFGIFVVDKNTGDINITAIVDREETPSFLITVRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQAEVNIKVKDVNDNFPMFRGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 47 - protein, 726 aa, synthetic construct
  REGION 1..726: note = Synthetic
EFRIQVRDYNTKNGTIKWHSIRRQKREAIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLPETG

SEQ ID NO: 48 - protein, 728 aa, synthetic construct
  REGION 1..728: note = Synthetic
ELRIETKGQYDEEEMTMQQAKRRQKREAVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 49 - protein, 614 aa, synthetic construct
  REGION 1..614: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLPETG

SEQ ID NO: 50 - protein, 612 aa, synthetic construct
  REGION 1..612: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 51 - protein, 499 aa, synthetic construct
  REGION 1..499: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLPETG

SEQ ID NO: 52 - protein, 497 aa, synthetic construct
  REGION 1..497: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 53 - protein, 499 aa, synthetic construct
  REGION 1..499: note = Synthetic
EFRIQVRDYNTKNGTIKWHSIRRQKREAIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLPETG

SEQ ID NO: 54 - protein, 497 aa, synthetic construct
  REGION 1..497: note = Synthetic
ELRIETKGQYDEEEMTMQQAKRRQKREAVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 55 - protein, 728 aa, synthetic construct
  REGION 1..728: note = Synthetic
ELRIETKGQYDEEEMTMQQAKRRQKREWVKFAKPSREGEDNSKRNPIAKITSDYQATQKITYRISGVGIDQPPFGIFVVDKNTGDINITAIVDREETPSFLITSRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQSESNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 56 - protein, 726 aa, synthetic construct
  REGION 1..726: note = Synthetic
EFRIQVRDYNTKNGTIKWHSIRRQKREWIKFAAASREGEDNSKRNPIAKIHSDSAANQQVTYRISGVGIDQPPYGIFVINQKTGEINITSIVDREVTPFFIIYSRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAESESNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 57 - protein, 728 aa, synthetic construct
  REGION 1..728: note = Synthetic
ELRIETKGQYDEEEMTMQQAKRRQKREWVKFAKPCREGEDNSKRNPIAKITSDYQATQKITYRISGVGIDQPPFGIFVVDKNTGDIQITAIVDREETPSFLITCRALNAQGLDVEKPLILTVKILDINDNPPVFSQQIFMGEIEENSASNSLVMILQATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 58 - protein, 726 aa, synthetic construct
  REGION 1..726: note = Synthetic
EFRIQVRDYNTKNGTIKWHSIRRQKREWIKFAAACREGEDNSKRNPIAKIHSDCAANQQVTYRISGVGIDQPPYGIFVINQKTGEIQITSIVDREVTPFFIIYCRALNSMGQDLERPLELRVRVLDINDNPPVFSMATFAGQIEENSNANTLVMILQATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNGGGGSGGGGSGGGGSEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 59 - protein, 499 aa, synthetic construct
  REGION 1..499: note = Synthetic
EFRIQVRDYNTKNGTIKWHSIRRQKREWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLPETG

SEQ ID NO: 60 - protein, 497 aa, synthetic construct
  REGION 1..497: note = Synthetic
ELRIETKGQYDEEEMTMQQAKRRQKREWVKFAKPVREGEDNSKRNPIAKITSDYQATQKITYRISGVGIDQPPFGIFVVDKNTGDINITAIVDREETPSFLITVRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQAEVNIKVKDVNDNFPMFRGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 61 - protein, 499 aa, synthetic construct
  REGION 1..499: note = Synthetic
EFRIQVRDYNTKNGTIKWHSIRRQKREWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKLPETG

SEQ ID NO: 62 - protein, 497 aa, synthetic construct
  REGION 1..497: note = Synthetic
ELRIETKGQYDEEEMTMQQAKRRQKREWVKFAKPVREGEDNSKRNPIAKITSDYQATQKITYRISGVGIDQPPFGIFVVDKNTGDINITAIVDREETPSFLITVRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQAEVNIKVKDVNDNFPMFRGGGGSGGGGSGGGGSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKLPETG

SEQ ID NO: 63 - protein, 476 aa, synthetic construct
  REGION 1..476: note = Synthetic
ELRIETKGQYDEEEMTMQQAKRRQKREAVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 64 - protein, 245 aa, synthetic construct
  REGION 1..245: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFR

SEQ ID NO: 65 - protein, 245 aa, synthetic construct
  REGION 1..245: note = Synthetic
ELRIETKGQYDEEEMTMQQAKRRQKREAVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFR

SEQ ID NO: 66 - protein, 476 aa, synthetic construct
  REGION 1..476: note = Synthetic
ELRIETKGQYDEEEMTMQQAKRRQKREWVKFAKPVREGEDNSKRNPIAKITSDYQATQKITYRISGVGIDQPPFGIFVVDKNTGDINITAIVDREETPSFLITVRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQAEVNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 67 - protein, 476 aa, synthetic construct
  REGION 1..476: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDRGETPSFLITCRALNAQGLDVEKPLILTVKILDINGNPPVFSQQIFMGEIEENSASNSLVMILNATDAGEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 68 - protein, 476 aa, synthetic construct
  REGION 1..476: note = Synthetic
ELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDRGQASSYRLVVSGADKDGEGLSTQCECNIKVKDVNGNFPMFRDSQYSARIEENILSSELLRFQVTDLGEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 69 - protein, 474 aa, synthetic construct
  REGION 1..474: note = Synthetic
EFRIQVRDYNTKNGTIKWHSIRRQKREAIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 70 - protein, 247 aa, synthetic construct
  REGION 1..247: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYME

SEQ ID NO: 71 - protein, 247 aa, synthetic construct
  REGION 1..247: note = Synthetic
EFRIQVRDYNTKNGTIKWHSIRRQKREAIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYME

SEQ ID NO: 72 - protein, 474 aa, synthetic construct
  REGION 1..474: note = Synthetic
EFRIQVRDYNTKNGTIKWHSIRRQKREWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 73 - protein, 474 aa, synthetic construct
  REGION 1..474: note = Synthetic
EFRIQVRDYNTKNGTIKWHSIRRQKREWIKFAAACREGEDNSKRNPIAKIHSDCAANQQVTYRISGVGIDQPPYGIFVINQKTGEINITSIVDRGVTPFFIIYCRALNSMGQDLERPLELRVRVLDINGNPPVFSMATFAGQIEENSNANTLVMILNATDAGEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 74 - protein, 448 aa, synthetic construct
  REGION 1..448: note = Synthetic
EAIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 75 - protein, 448 aa, synthetic construct
  REGION 1..448: note = Synthetic
EWIKFAAACREGEDNSKRNPIAKIHSTCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 76 - protein, 448 aa, synthetic construct
  REGION 1..448: note = Synthetic
EWIKFAAACREGEDNSKRNPIAKIHSDCAANQQVTYRISGVGIDQPPYGIFVINQKTGEINITSIVDREVTPFFIIYCRALNSMGQDLSRPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 77 - protein, 448 aa, synthetic construct
  REGION 1..448: note = Synthetic
EAIKFAAACREGEDNSKRNPIAKIHSTCAANQQVTYRISGVGIDQPPYGIFVINQKTGEINITSIVDREVTPFFIIYCRALNSMGQDLSRPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 78 - protein, 448 aa, synthetic construct
  REGION 1..448: note = Synthetic
EAIKFAAACREGEDNSKRNPIAKIHSDCAANQQVTYRISGVGIDQPPYGIFVINQKTGEINITSIVDREVTPFFIIYCRILNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 79 - protein, 448 aa, synthetic construct
  REGION 1..448: note = Synthetic
EWIKFAAACREGEDNSAANPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 80 - protein, 448 aa, synthetic construct
  REGION 1..448: note = Synthetic
EWIKFAAACAEGEDNSAANPIAKIHSDCAANQQVTYRISGVGIDQPPYGIFVINQKTGEINITSIVDREVTPFFIIYCRALNSMGQDLERPLELRVAVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 81 - protein, 448 aa, synthetic construct
  REGION 1..448: note = Synthetic
EWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTSTGTININIQSFGNDDRTNTEPN

SEQ ID NO: 82 - protein, 448 aa, synthetic construct
  REGION 1..448: note = Synthetic
EWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 83 - protein, 448 aa, synthetic construct
  REGION 1..448: note = Synthetic
EWIKFAAACREGEDNSKRNPIAKIHSDCAANQQVTYRISGVGIDQPPYGIFVINQKTGEINITSIVDRGVTPFFIIYCRALNSMGQDLERPLELRVRVLDINGNPPVFSMATFAGQIEENSNANTLVMILNATDAGEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 84 - protein, 448 aa, synthetic construct
  REGION 1..448: note = Synthetic
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDRGQYGQYALAVRGSDRDGGADGMSAECECNIKILDVNGNIPYMEQSSYTIEIQENTLNSNLLEIRVIDLGEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 85 - protein, 448 aa, synthetic construct
  REGION 1..448: note = Synthetic
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQGNTLNSNLLEIRVIDLDEEFSANWMAVIFFISGNEGNWFEIEMNERTNVGILKVVKPLDYGAMQSLQLSIGVRNKAEFHHSIMSQYKLKASAISVTVLNVIGGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 86 - protein, 336 aa, synthetic construct
  REGION 1..336: note = Synthetic
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVF

SEQ ID NO: 87 - protein, 221 aa, synthetic construct
  REGION 1..221: note = Synthetic
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYME

SEQ ID NO: 88 - protein, 339 aa, synthetic construct
  REGION 1..339: note = Synthetic
MATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 89 - protein, 227 aa, synthetic construct
  REGION 1..227: note = Synthetic
QSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 90 - protein, 221 aa, synthetic construct
  REGION 1..221: note = Synthetic
EWIKFAAAVREGEDNSKRNPIAKIHSDAAANQQVTYRISGVGIDQPPYGIFVINQKTGEINITSIVDREVTPFFIIYVRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAEAEVNIKILDVNDNIPYME

SEQ ID NO: 91 - protein, 221 aa, synthetic construct
  REGION 1..221: note = Synthetic
EAIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYME

SEQ ID NO: 92 - protein, 448 aa, synthetic construct
  REGION 1..448: note = Synthetic
EWIKFAAASREGEDNSKRNPIAKIHSDSAANQQVTYRISGVGIDQPPYGIFVINQKTGEINITSIVDREVTPFFIIYSRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAESESNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 93 - protein, 448 aa, synthetic construct
  REGION 1..448: note = Synthetic
EWIKFAAACREGEDNSKRNPIAKIHSDCAANQQVTYRISGVGIDQPPYGIFVINQKTGEIQITSIVDREVTPFFIIYCRALNSMGQDLERPLELRVRVLDINDNPPVFSMATFAGQIEENSNANTLVMILQATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 94 - protein, 450 aa, synthetic construct
  REGION 1..450: note = Synthetic
EAVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 95 - protein, 450 aa, synthetic construct
  REGION 1..450: note = Synthetic
EWVKFAKPCREGEDNSKRNPIAKITSDYQATQKITYRISGVGIDQPPFGIFVVDKNTGDINITAIVDREETPSFLITCRALNAQGLDVSKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 96 - protein, 450 aa, synthetic construct
  REGION 1..450: note = Synthetic
EAVKFAKPCREGEDNSKRNPIAKITSTYQATQKITYRISGVGIDQPPFGIFVVDKNTGDINITAIVDREETPSFLITCRALNAQGLDVSKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 97 - protein, 450 aa, synthetic construct
  REGION 1..450: note = Synthetic
EAVKFAKPCREGEDNSKRNPIAKITSDYQATQKITYRISGVGIDQPPFGIFVVDKNTGDINITAIVDREETPSFLITCRILNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 98 - protein, 450 aa, synthetic construct
  REGION 1..450: note = Synthetic
EWVKFAKPCREGEDNSAANPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 99 - protein, 450 aa, synthetic construct
  REGION 1..450: note = Synthetic
EWVKFAKPCAEGEDNSAANPIAKITSDYQATQKITYRISGVGIDQPPFGIFVVDKNTGDINITAIVDREETPSFLITCRALNAQGLDVEKPLILTVAILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 100 - protein, 450 aa, synthetic construct
  REGION 1..450: note = Synthetic
EWVKFAKPVREGEDNSKRNPIAKITSDYQATQKITYRISGVGIDQPPFGIFVVDKNTGDINITAIVDREETPSFLITVRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQAEVNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 101 - protein, 450 aa, synthetic construct
  REGION 1..450: note = Synthetic
EWVKFAKPCREGEDNSKRNPIAKITSDYQATQKITYRISGVGIDQPPFGIFVVDKNTGDINITAIVDRGETPSFLITCRALNAQGLDVEKPLILTVKILDINGNPPVFSQQIFMGEIEENSASNSLVMILNATDAGEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 102 - protein, 450 aa, synthetic construct
  REGION 1..450: note = Synthetic
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDRGQASSYRLVVSGADKDGEGLSTQCECNIKVKDVNGNFPMFRDSQYSARIEENILSSELLRFQVTDLGEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 103 - protein, 450 aa, synthetic construct
  REGION 1..450: note = Synthetic
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEGNILSSELLRFQVTDLDEEYTDNWLAVYFFTSGNEGNWFEIQTDPRTNEGILKVVKALDYGQLQSVKLSIAVKNKAEFHQSVISRYRVQSTPVTIQVINVRGGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 104 - protein, 334 aa, synthetic construct
  REGION 1..334: note = Synthetic
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAF

SEQ ID NO: 105 - protein, 219 aa, synthetic construct
  REGION 1..219: note = Synthetic
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFR

SEQ ID NO: 106 - protein, 341 aa, synthetic construct
  REGION 1..341: note = Synthetic
QQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 107 - protein, 231 aa, synthetic construct
  REGION 1..231: note = Synthetic
DSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 108 - protein, 219 aa, synthetic construct
  REGION 1..219: note = Synthetic
EWVKFAKPVREGEDNSKRNPIAKITSDYQATQKITYRISGVGIDQPPFGIFVVDKNTGDINITAIVDREETPSFLITVRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQAEVNIKVKDVNDNFPMFR

SEQ ID NO: 109 - protein, 219 aa, synthetic construct
  REGION 1..219: note = Synthetic
EAVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFR

SEQ ID NO: 110 - protein, 450 aa, synthetic construct
  REGION 1..450: note = Synthetic
EWVKFAKPSREGEDNSKRNPIAKITSDYQATQKITYRISGVGIDQPPFGIFVVDKNTGDINITAIVDREETPSFLITSRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQSESNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 111 - protein, 450 aa, synthetic construct
  REGION 1..450: note = Synthetic
EWVKFAKPCREGEDNSKRNPIAKITSDYQATQKITYRISGVGIDQPPFGIFVVDKNTGDIQITAIVDREETPSFLITCRALNAQGLDVEKPLILTVKILDINDNPPVFSQQIFMGEIEENSASNSLVMILQATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 112 - protein, 450 aa, synthetic construct; identical to SEQ ID NO: 101
  REGION 1..450: note = Synthetic

SEQ ID NO: 113 - protein, 450 aa, synthetic construct; identical to SEQ ID NO: 102
  REGION 1..450: note = Synthetic

SEQ ID NO: 114 - protein, 109 aa, Homo sapiens
EWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFS

SEQ ID NO: 115 - protein, 112 aa, Homo sapiens
MATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYME

SEQ ID NO: 116 - protein, 115 aa, Homo sapiens
QSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVF

SEQ ID NO: 117 - protein, 112 aa, Homo sapiens
RPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPN

SEQ ID NO: 118 - protein, 109 aa, Homo sapiens
EWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFS

SEQ ID NO: 119 - protein, 110 aa, Homo sapiens
QQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFR

SEQ ID NO: 120 - protein, 115 aa, Homo sapiens
DSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAF

SEQ ID NO: 121 - protein, 116 aa, Homo sp.
RPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEK

SEQ ID NO: 122 - protein, 19 aa, Homo sapiens
MEWSWVFLFFLSVTTGVHS

SEQ ID NO: 123 - protein, 20 aa, Homo sapiens
MSVPTQVLGLLLLWLTDARC

SEQ ID NO: 124 - protein, 15 aa, Homo sapiens
EPKSSDKTHTCPPCP

SEQ ID NO: 125 - protein, 23 aa, Homo sapiens
EPKSCDKTHTCPPCPAPELLGGP

SEQ ID NO: 126 - protein, 19 aa, Homo sapiens
EPKCCVECPPCPAPPAAAP

SEQ ID NO: 127 - protein, 20 aa, Homo sapiens
ESKYGPPCPPCPAPEFLGGP

SEQ ID NO: 128 - protein, 70 aa, Homo sapiens
ELKTPLGDTTHTCPRCPEPKSCDTPPPCPRCPEPKSCDTPPPCPRCPEPKSCDTPPPCPRCPAPELLGGP

SEQ ID NO: 129 - protein, 5 aa, synthetic construct
  REGION 1..5: note = Synthetic
  SITE 3: note = misc_feature - Xaa is selected from P and R
CPXCP

SEQ ID NO: 130 - protein, 5 aa, synthetic construct
  REGION 1..5: note = Synthetic
CPPCP

SEQ ID NO: 131 - protein, 5 aa, synthetic construct
  REGION 1..5: note = Synthetic
CPRCP

SEQ ID NO: 132 - protein, 15 aa, Homo sapiens
EPKSCDKTHTCPPCP

SEQ ID NO: 133 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 134 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 135 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 136 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 137 - protein, 104 aa, Homo sapiens
AAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 139 - protein, 102 aa, Homo sapiens
SVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 140 - protein, 102 aa, Homo sapiens
SVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 141 - protein, 102 aa, Homo sapiens
SVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 142 - protein, 102 aa, Homo sapiens
SVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 143 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 107 aa, Homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 146 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 148 - protein, 107 aa, synthetic construct
  REGION 1..107: note = Synthetic
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 107 aa, synthetic construct
  REGION 1..107: note = Synthetic
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 150 - protein, 107 aa, synthetic construct
  REGION 1..107: note = Synthetic
GQPREPQVYTLPPSREEMTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 107 aa, synthetic construct
  REGION 1..107: note = Synthetic
GQPREPQVYTLPPSREEMTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 152 - protein, 5 aa, synthetic construct
  REGION 1..5: note = Synthetic
GGGGS

SEQ ID NO: 153 - protein, 25 aa, synthetic construct
  REGION 1..25: note = Synthetic
GGGGSGGGGSGGGGSGGGGSGGGGS

SEQ ID NO: 154 - protein, 12 aa, synthetic construct
  REGION 1..12: note = Synthetic
GSAGSAAGSGEF

SEQ ID NO: 155 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 157 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 23 aa, Homo sapiens
MDWSFFRVVAMLFIFLVVVEVNS

SEQ ID NO: 159 - protein, 23 aa, Homo sapiens
MMGLFPRTTGALAIFVVVILVHG

SEQ ID NO: 160 - protein, 19 aa, synthetic construct
  REGION 1..19: note = Synthetic
MEFGLSWLFLVAILKGVQC

SEQ ID NO: 161 - protein, 19 aa, synthetic construct
  REGION 1..19: note = Synthetic
MGWSCIILFLVATATGVHS

SEQ ID NO: 162 - protein, 22 aa, synthetic construct
EPKSCDKTHTCPPCPAPEAAGG

SEQ ID NO: 163 - protein, 102 aa, synthetic construct
SVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAK

SEQ ID NO: 164 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 232 aa, synthetic construct
EPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 167 - protein, 232 aa, synthetic construct
EPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 232 aa, synthetic construct
EPKSCDKTHTCPPCPAPELLAGPSVFLFPPKPKDTLMISRTPEVTCVVVDVEFEDPEVKFNWYVDGVEVHNAKTKPREEQYNSTYRVVSVLTVLHQDWLNGKEYKCKVTNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 232 aa, synthetic construct
EPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 5 aa, synthetic construct
EAAAK

SEQ ID NO: 171 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK